(19) United States
(12) Patent Application Publication
(10) Pub. No.: US 2024/0109842 A1
(43) Pub. Date: Apr. 4, 2024

(54) ACRYLAMIDE COMPOUNDS

(71) Applicant: OTSUKA PHARMACEUTICAL CO., LTD., Tokyo (JP)

(72) Inventors: Hideki HAYASHI, Osaka-shi, Osaka (JP); Ryosuke TAGA, Osaka-shi, Osaka (JP); Yuki SAKAMOTO, Osaka-shi, Osaka (JP); Nozomi KUWANO, Osaka-shi, Osaka (JP); Kurumi MINENO, Osaka-shi, Osaka (JP); Kazuhiro OHDACHI, Osaka-shi, Osaka (JP); Yusuke FUJIMORI, Osaka-shi, Osaka (JP)

(73) Assignee: OTSUKA PHARMACEUTICAL CO., LTD., Tokyo (JP)

(21) Appl. No.: 17/766,948

(22) PCT Filed: Dec. 8, 2020
(86) PCT No.: PCT/JP2020/045713

§ 371 Date: Apr. 6, 2022

(30) Foreign Application Priority Data
Dec. 9, 2019 (JP) 2019-222190
Jan. 28, 2020 (JP) 2020-011573

(51) Int. Cl. C07D 209/18; C07D 209/26; C07D 235/16; C07D 401/12; C07D 403/12; C07D 405/12; C07D 409/12; C07D 409/14; C07D 413/12; C07D 417/12; C07D 471/04; C12N 5/078

(52) U.S. Cl. CPC C07D 209/18; C07D 209/26; C07D 235/16; C07D 401/12; C07D 403/12; C07D 405/12; C07D 409/12; C07D 409/14; C07D 413/12; C07D 417/12; C07D 471/04; C12N 5/0644; C12N 2501/999

(57) ABSTRACT

Provided is an acrylamide compound, which is useful for the promotion of platelet production from platelet progenitor cells such as megakaryocytes in vitro and represented by general formula [I]:

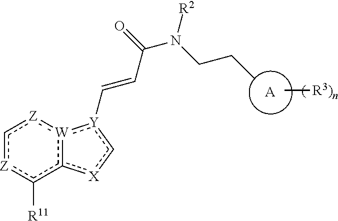

wherein each symbol is as defined in the description.

### TECHNICAL FIELD

The present invention relates to an acrylamide compound. More specifically, the present invention relates to an acrylamide compound promoting platelet production from platelet progenitor cells such as megakaryocytes in vitro.

### BACKGROUND ART

Platelet preparations are administrated to patients who suffer from massive bleeding during surgery or injury, or tend to bleed due to decrease of platelets after treatment with an anti-cancer agent for treatment and/or prevention of unexpected bleeding.

Currently, the platelet preparations rely on the donation of blood, and the shelf life is about 4 days, which is extremely short. Further, as long as the platelet preparations are supplied only by the donation of blood, it is expected that reduction of blood donors may lead to shortage of platelet preparations in the near future.

In order to meet these needs, a method for producing platelets in vitro has been studied.

As the method for producing platelets in vitro, a method for obtaining megakaryocytes by differentiating various types of stem cells followed by the culturing thereof to release platelets into the medium has been developed. Takayama, et al., for example, have succeeded in inducing human ES cells to differentiate into megakaryocytes and platelets (NPL 1).

In addition, as a method for producing platelets from hematopoietic progenitor cells in vitro, a method of culturing hematopoietic progenitor cells in the presence of an aryl hydrocarbon receptor antagonist and thrombopoietin (TPO) or a Rho-associated coiled-coil forming kinase (ROCK) inhibitor has been proposed (PTL 1, 2 and 3, and NPL 2, 3 and 4).

Indolyl acrylamide compounds has been reported as a transcription factor inhibitor (PTL 4 and NPL 5)

### CITATION LIST

### Patent Literature

[PTL 1] WO 2014/138485
[PTL 2] WO 2016/204256
[PTL 3] WO 2010/059401
[PTL 4] WO 2019/167973

### Non Patent Literature

[NPL 1] Takayama et al., Blood, 111, 5298 (2008)
[NPL 2] Boitano et al., Science, 329, 1345 (2010)
[NPL 3] Strassel et al., Blood, 127, 2231 (2016)
[NPL 4] Ito et al., Cell, 174, 636 (2018)
[NPL 5] Perron et al., J. Biol. Chem., 293, 8285 (2018)

### SUMMARY OF INVENTION

### Technical Problem

An object of the present invention is to provide a novel acrylamide compound or a salt thereof, which is useful for the promotion of platelet production from platelet progenitor cells such as megakaryocytes in vitro.

Another object of the present invention is to provide a platelet production promoting agent, which is useful for the promotion of platelet production from platelet progenitor cells such as megakaryocytes in vitro.

### Solution to Problem

As a result of conducting extensive studies to solve the above-mentioned problems, the inventors of the present invention found that the acrylamide compound represented by the following formula [I] or [I′] has an effect of promoting platelet production, thereby leading to completion of the present invention.

Namely, the present invention includes the following embodiments.

[1-1] A compound represented by general formula [I]:

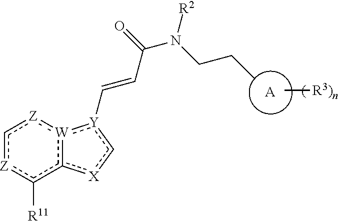

wherein
   R¹¹ is hydrogen, halogen, —C₁₋₆ alkyl or —O—C₁₋₆ alkyl;
   R² is hydrogen or —C₁₋₆ alkyl,
   R³ is halogen, -Qₖ-(C₁₋₆ alkyl)ₘ-Qₚ-R³¹, optionally-substituted phenyl or optionally-substituted heteroaryl which is selected from the groupe consisting of furyl, thienyl, oxazolyl, thiazolyl, pyrazolyl, pyridyl, pyrazyl, pyridazinyl and pyrimidyl,
   R³¹ is —C₁₋₆ alkyl or —C₃₋₈ cycloalkyl,
   Qs are the same or different and each independently represent oxygen, sulfur, —C(═O)—O— or —NH—,
   k, m and p are 0 or 1,
   n is 0, 1 or 2, wherein when n is 2, R³s each independently represent the same or different substituent,
   W is carbon or nitrogen,
   X is carbon, nitrogen or N—R¹²,
   Y is carbon or nitrogen,
   Zs are the same or different and each independently represent nitrogen or C—H,
      provided that X and Y are not carbon at the same time
      R¹² is hydrogen, —C₁₋₆ alkyl, —C₁₋₆ alkyl-O—C₁₋₆ alkyl, —C(═O)—C₁₋₆ alkyl, —C(═O)-aryl or —C(═O)—O—C₁₋₆ alkyl,
   Ring A is aryl or heteroaryl,
   [symbol] is single bond or double bond,
      provided that when X is N—H, W and Y are carbon and all Z are C—H, ring A is neither 2-(-O—C₁₋₆ alkyl)phenyl nor 2,5-di (—O—C₁₋₆ alkyl)phenyl,
or a salt thereof.

[1-2] The compound according to [1-1], wherein in the general formula [I],

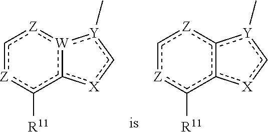

wherein R¹¹, W, X, Y, Zs and [symbol] are as defined above,
or a salt thereof.

[1-3] The compound according to [1-1], wherein in the general formula [I],

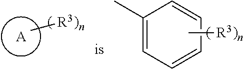

wherein R³ and n are as defined above,
or a salt thereof.

[1-4] The compound according to [1-1], wherein in the general formula [I], the heteroaryl in Ring A is selected from the group consisting of furan, thiophene, pyridine and quinoline, or a salt thereof.

[1-5] The compound according to [1-1], wherein in the general formula [I],

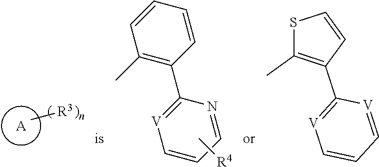

wherein Vs are the same or different and each independently represent nitrogen or C—H, R⁴ is hydrogen, halogen, —C₁₋₆ alkyl or —O—C₁₋₆ alkyl,
or a salt thereof.

[1-6] The compound according to [1-1], which is represented by general formula [Ia]:

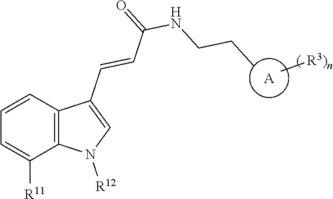

wherein R¹¹ is hydrogen, halogen, —C₁₋₆ alkyl or —O—C₁₋₆ alkyl,
   R¹² is hydrogen or —C(═O)—O—C₁₋₆ alkyl,

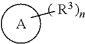

is pyridylbenzene, pyrimidylbenzene (wherein the pyrimidyl is optionally substituted by halogen, —C₁₋₆ alkyl or —O—C₁₋₆ alkyl), phenylthiophene, pyridylthiophene or pyrimidylthiophene, or a salt thereof.

[1-7] The compound according to [1-1], which is selected from the group consisting of the following compounds:

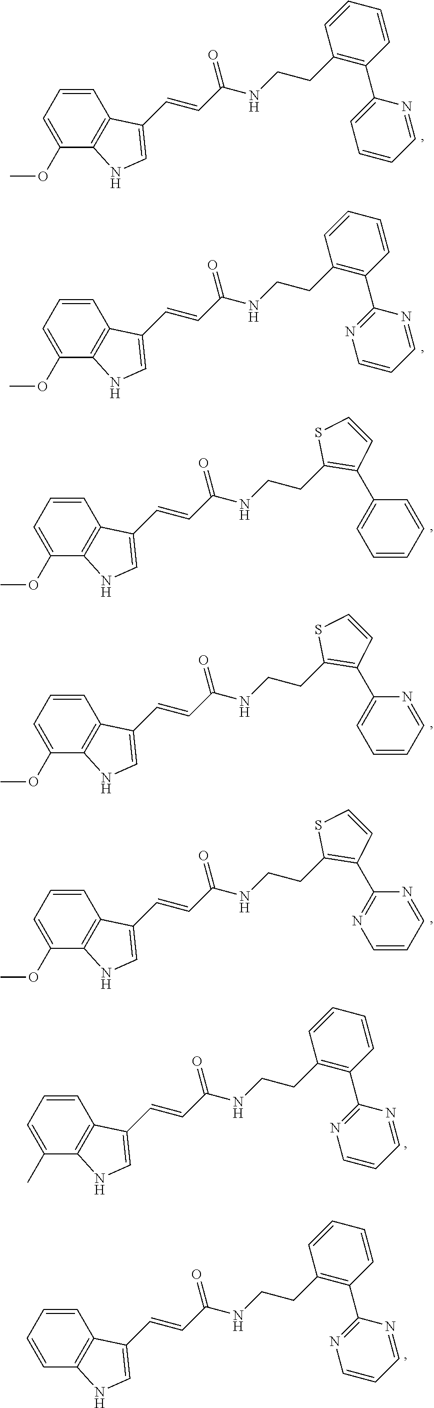

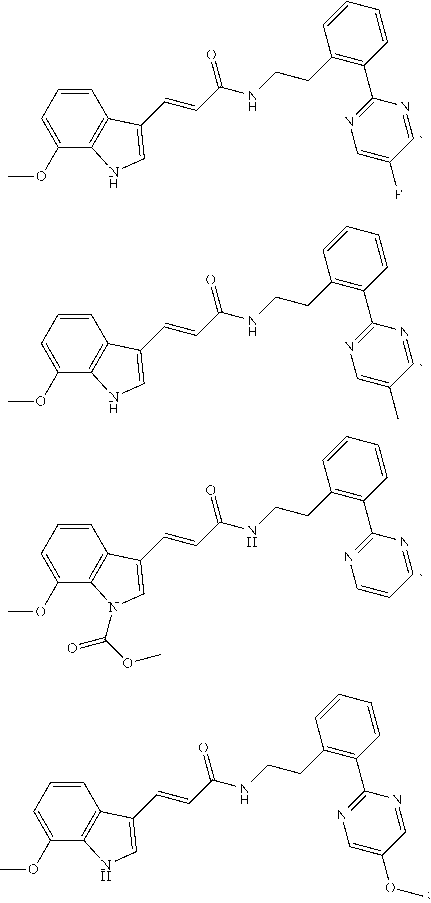

or a salt thereof.

[2-1] A platelet production promoting agent comprising a compound represented by general formula [I′]:

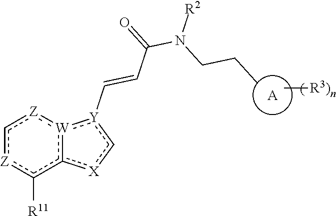

wherein
   R¹¹ is hydrogen, halogen, —C₁₋₆ alkyl or —O—C₁₋₆ alkyl;
   R² is hydrogen or —C₁₋₆ alkyl,
   R³ is halogen, -Qₖ-(C₁₋₆ alkyl)ₘ-Qₚ-R³¹, optionally-substituted phenyl or optionally-substituted heteroaryl which is selected from the groupe consisting of furyl, thienyl, oxazolyl, thiazolyl, pyrazolyl, pyridyl, pyrazyl, pyridazinyl and pyrimidyl,
   R³¹ is —C₁₋₆ alkyl or —C₃₋₈ cycloalkyl,
   Qs are the same or different and each independently represent oxygen, sulfur, —C(═O)—O— or —NH—,
   k, m and p are 0 or 1,
   n is 0, 1 or 2, wherein when n is 2, R³s each independently represent the same or different substituent,
   W is carbon or nitrogen,
   X is carbon, nitrogen or N—R¹²,
   Y is carbon or nitrogen,
   Zs are the same or different and each independently represent nitrogen or C—H,
      provided that X and Y are not carbon at the same time
      R¹² is hydrogen, —C₁₋₆ alkyl, —C₁₋₆ alkyl-O—C₁₋₆ alkyl, —C(═O)—C₁₋₆ alkyl, —C(═O)-aryl or —C(═O)—O—C₁₋₆ alkyl,
   Ring A is aryl or heteroaryl,
   [symbol] is single bond or double bond,
or a salt thereof.

[2-2] The platelet production promoting agent according to [2-1], comprising the compound, wherein in the general formula [I′],

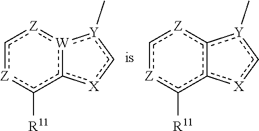

wherein R¹¹, X, Y, W, Zs and [symbol] are as defined above,
or a salt thereof.

[2-3] The platelet production promoting agent according to [2-1], comprising the compound, wherein in the general formula

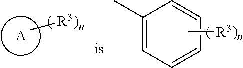

wherein R³ and n are as defined above,
or a salt thereof.

[2-4] The platelet production promoting agent according to [2-1], comprising the compound, wherein in the general formula [I′], the heteroaryl in Ring A is selected from the group consisting of furan, thiophene, pyridine and quinoline,
or a salt thereof.

[2-5] The platelet production promoting agent according to [2-1], comprising the compound, wherein in the general formula [I′],

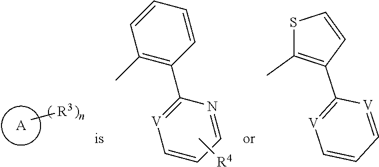

wherein Vs are the same or different and each independently represent nitrogen or C—H, R⁴ is hydrogen, halogen, —C₁₋₆ alkyl or —O—C₁₋₆ alkyl,
or a salt thereof.

[2-6] The platelet production promoting agent according to [2-1], comprising the compound, which is represented by general formula [Ia]:

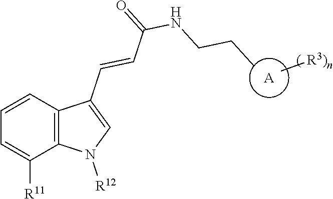

wherein R is hydrogen, halogen, —C₁₋₆ alkyl or —O—C₁₋₆ alkyl,
   R¹² is hydrogen or —C(═O)—O—C₁₋₆ alkyl,

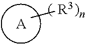

is pyridylbenzene, pyrimidylbenzene (wherein the pyrimidyl is optionally substituted by halogen, —C₁₋₆ alkyl or —O—C₁₋₆ alkyl), phenylthiophene, pyridylthiophene or pyrimidylthiophene, or a salt thereof.

[2-7] The platelet production promoting agent according to [2-1], comprising the compound, which is selected from the group consisting of the following compounds:

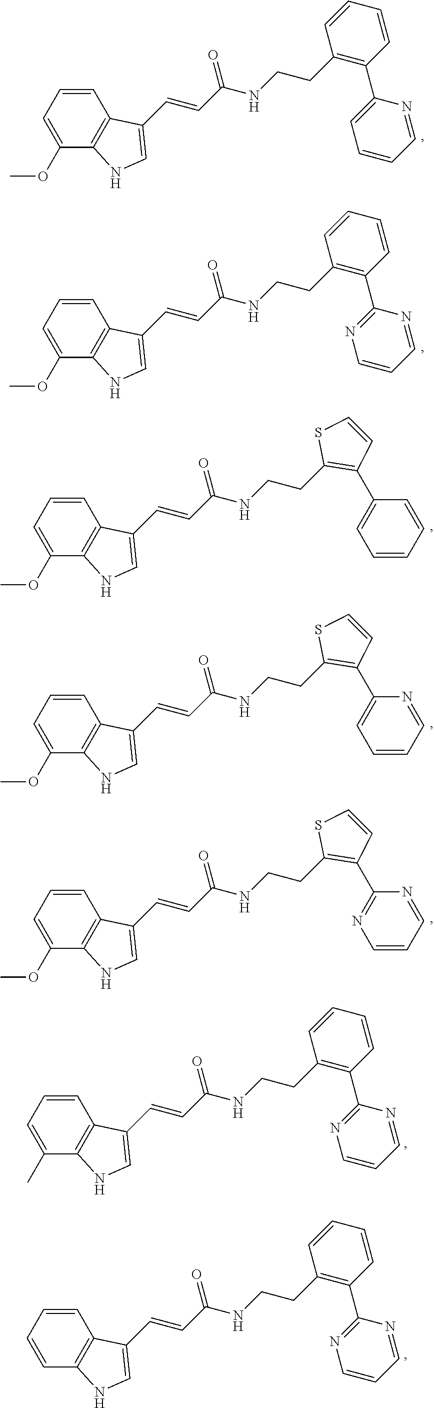

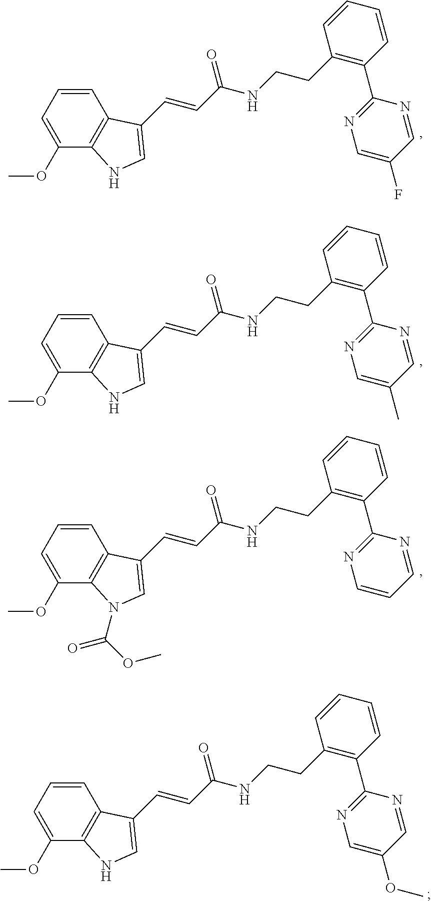

or a salt thereof.

[2-8] A platelet production promoting agent comprising a compound represented by general formula [Ia′]

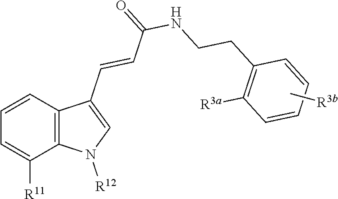

wherein
   R^{3a} is —O—C₁₋₆ alkyl;
   R^{3b} is hydrogen or —O—C₁₋₆ alkyl;
   R¹¹ is —C₁₋₆ alkyl or —O—C₁₋₆ alkyl;
   R¹² is hydrogen or —C₁₋₆ alkyl,
or a salt thereof.

[2-9] The platelet production promoting agent according to [2-8], comprising the compound, wherein in the general formula [Ia′],
R^{3a} is —O-methyl or —O-ethyl;
R^{3b} is hydrogen or —O-methyl;
R¹¹ is methyl or —O-methyl;
R¹² is hydrogen or methyl,
or a salt thereof.

[2-10] A platelet production promoting agent according to [2-8], comprising the compound, which is selected from the group consisting of the following compounds:

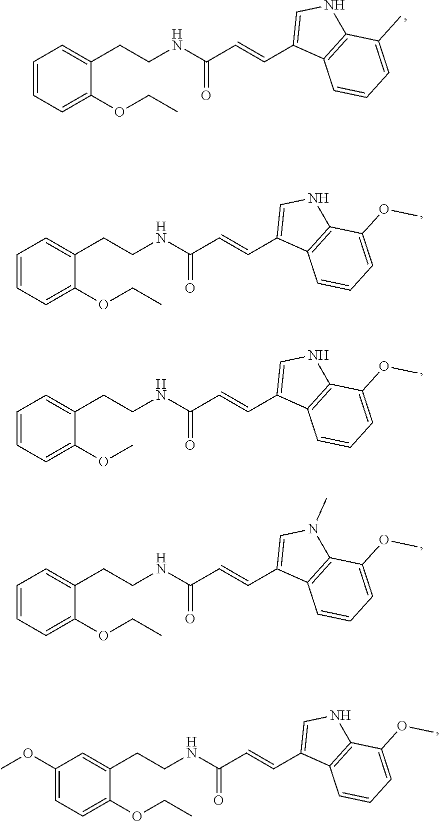

or a salt thereof.

[2-11] The platelet production promoting agent according to any one of [2-1] to [2-10], which is for use in combination with an aryl hydrocarbon receptor antagonist.

[2-12] The platelet production promoting agent according to [2-11], wherein the aryl hydrocarbon receptor antagonist is selected from the group consisting of the following compounds:

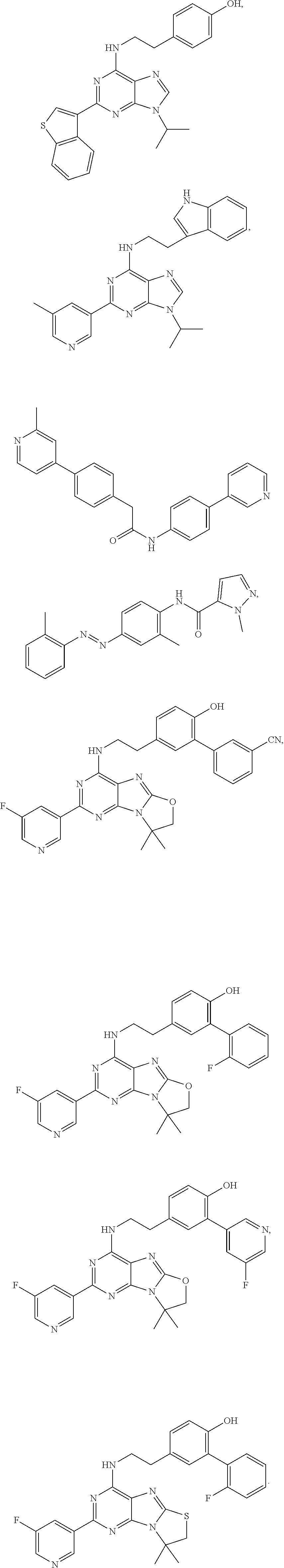

[3-1] Use of the compound according to any one of [2-1] to [2-10] or a salt thereof for promoting platelet production.

[3-2] The use according to [3-1], wherein the compound or a salt thereof is used in combination with an aryl hydrocarbon receptor antagonist.

[3-3] The use according to [3-2], wherein the aryl hydrocarbon receptor antagonist is selected from the group consisting of the following compounds:

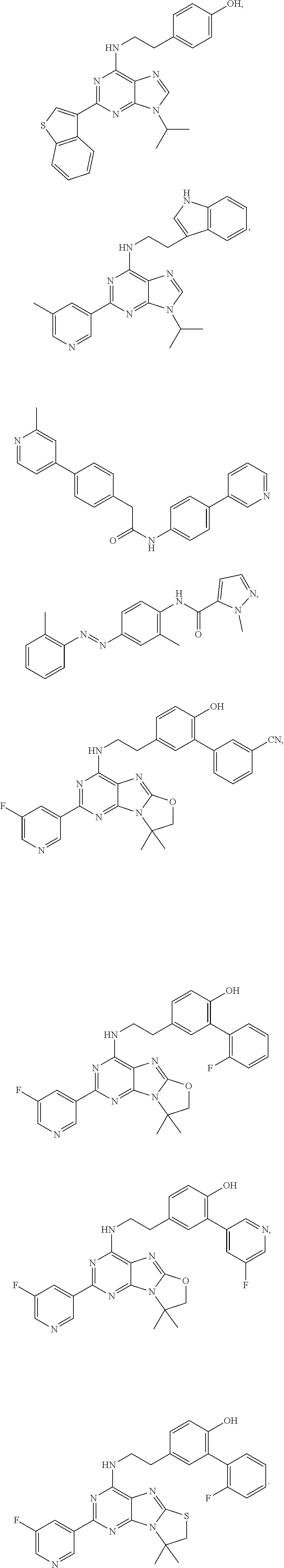

[4-1] The compound according to any one of [2-1] to [2-10] or a salt thereof for use in promoting platelet production.

[4-2] The compound according to [4-1] or a salt thereof, which is used in combination with an aryl hydrocarbon receptor antagonist.

[4-3] The compound according to [4-2] or a salt thereof, wherein the aryl hydrocarbon receptor antagonist is selected from the group consisting of the following compounds:

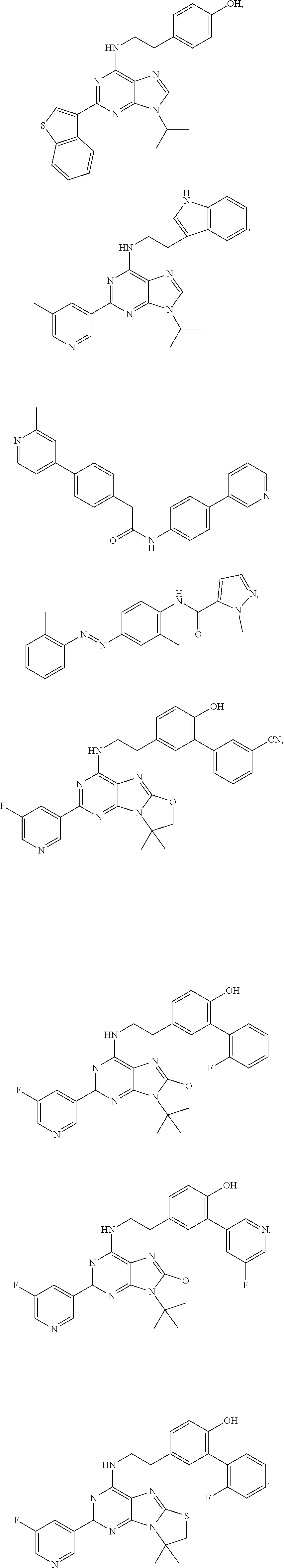

[5-1] A method for promoting platelet production, which comprises culturing platelet progenitor cells in the presence of the compound according to any one of [2-1] to [2-10] or a salt thereof.

[5-2] The method according to [5-1], wherein the compound or a salt thereof is used in combination with an aryl hydrocarbon receptor antagonist.

[5-3] The method according to [5-2], wherein the aryl hydrocarbon receptor antagonist is selected from the group consisting of the following compounds:

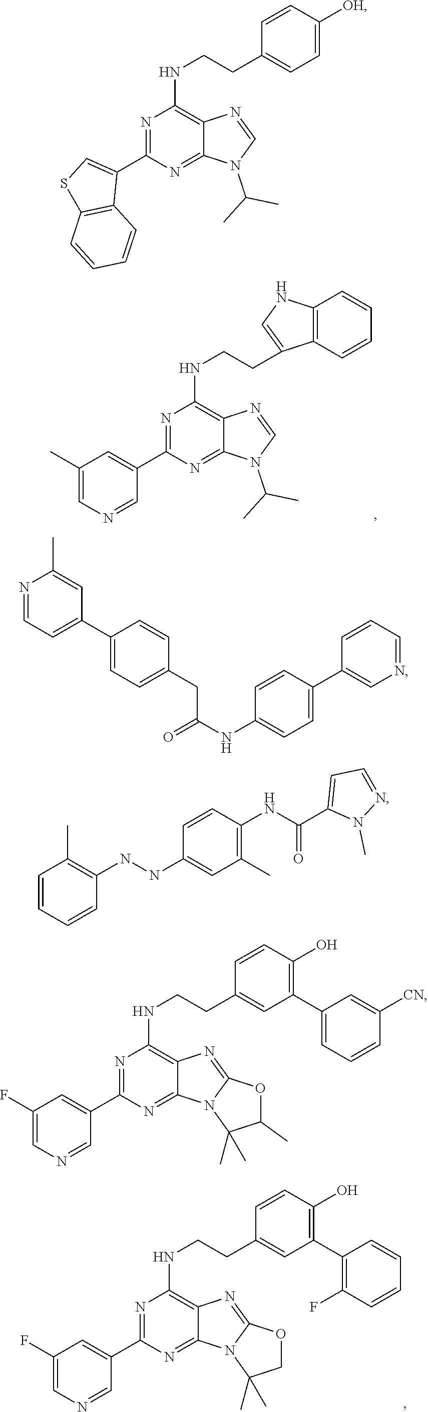

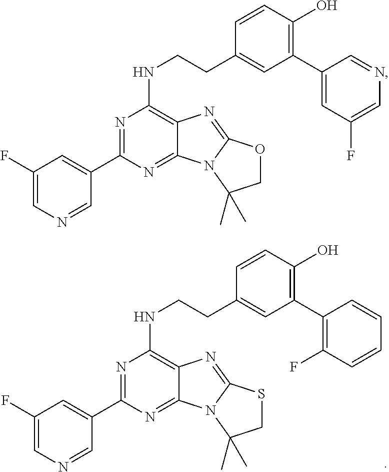

[6-1] A method for producing platelets, which comprises culturing platelet progenitor cells in the presence of the compound according to any one of [2-1] to [2-10] or a salt thereof.

[6-2] The method according to [6-1], which comprises culturing platelet progenitor cells in the copresence of an aryl hydrocarbon receptor antagonist.

[6-3] The method according to [6-2], wherein the aryl hydrocarbon receptor antagonist is selected from the group consisting of the following compounds:

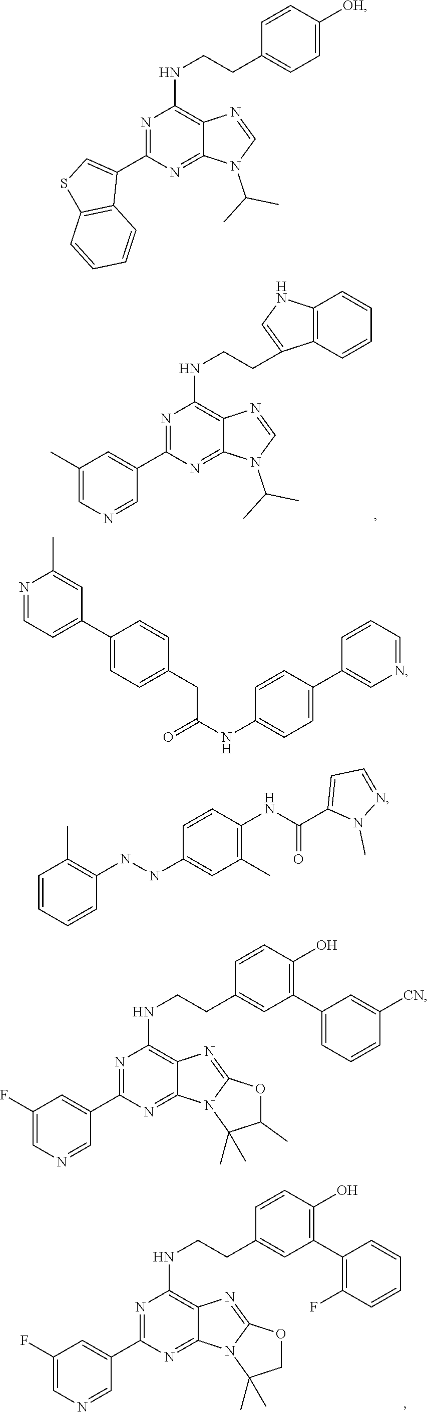

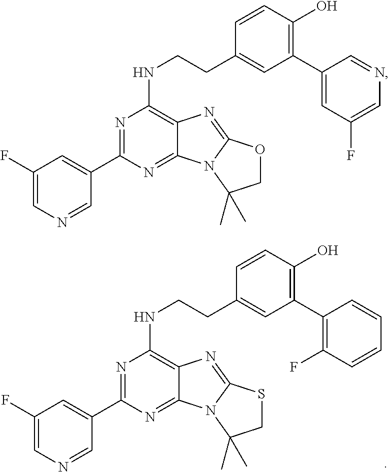

[7-1] A method for culturing platelet progenitor cells to promote platelet production, which comprises culturing platelet progenitor cells in the presence of the compound according to any one of [2-1] to [2-10] or a salt thereof.

[7-2] The method according to [7-1], which comprises culturing platelet progenitor cells in the copresence of an aryl hydrocarbon receptor antagonist.

[7-3] The method according to [7-2], wherein the aryl hydrocarbon receptor antagonist is selected from the group consisting of the following compounds:

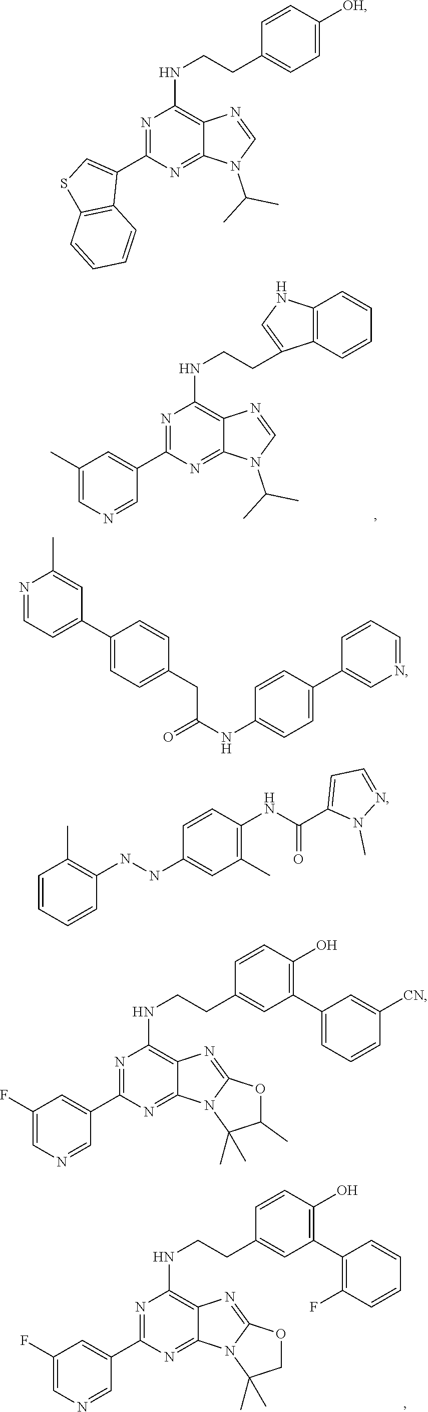

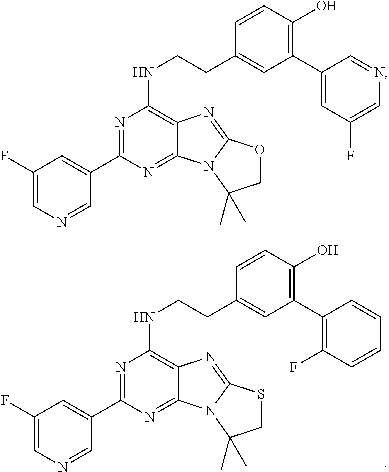

### Advantageous Effects of Invention

The compound or a salt thereof of the present invention has an excellent efficacy of promoting platelet production from platelet progenitor cells in vitro.

### DESCRIPTION OF EMBODIMENTS

The terms and phrases used in the present description will be described in detail below.

In the present description, “halogen” is fluorine, chlorine, bromine, or iodine. It is preferably fluorine, chlorine, or bromine, and more preferably fluorine or chlorine.

In the present description, “C₁₋₆ alkyl” is linear or branched alkyl having 1 to 6 carbon atoms (C₁₋₆), and specific examples thereof include methyl, ethyl, n-propyl, isopropyl, n-butyl, isobutyl, sec-butyl, tert-butyl, n-pentyl, isopentyl, neopentyl, n-hexyl, isohexyl, 3-methylpentyl, and the like.

In addition, the “C₁₋₆ alkyl” includes C₁₋₆ alkyl in which 1 to 7 hydrogen atoms are substituted by deuterium atoms.

In the present description, “C₃₋₈ cycloalkyl” is cycloalkyl having 3 to 8 carbon atoms (C₃₋₈), and specific examples thereof include cyclopropyl, cyclobutyl, cyclopentyl, cyclohexyl, cycloheptyl, cyclooctyl, and the like.

In the present description, “aryl” is monocyclic or polycyclic aromatic ring, and specific examples thereof include benzene, naphthalene, anthracene, and the like.

In the present description, “heteroaryl” is heterocyclic aromatic ring containing 1 to 3 hereroatoms independently selected from the group consisting of nitrogen, oxygen and sulfur as ring constituting atom, and specific examples thereof include furan, thiophene, oxazole, thiazole, pyrazole, pyridine, pyrimidine, pyridazine, pyrazine, quinoline, isoquinoline, quinazoline, and the like.

In the present description, “optionally-substituted phenyl” is an unsubstituted phenyl or a phenyl substituted by 1 to 3 substituents. Examples of the substituent include halogen, —C₁₋₆ alkyl, —O—C₁₋₆ alkyl, and the like. Specific examples of the “optionally-substituted phenyl” include phenyl, fluorophenyl, chlorophenyl, bromophenyl, iodophenyl, and the like.

In the present description, “optionally-substituted heteroaryl which is selected from the group consisting of furyl, thienyl, oxazolyl, thiazolyl, pyrazolyl, pyridyl, pyrazyl, pyridazinyl and pyrimidyl” is an unsubstituted furyl, thienyl, oxazolyl, thiazolyl, pyrazolyl, pyridyl, pyrazyl, pyridazinyl or pyrimidyl, or a furyl, thienyl, oxazolyl, thiazolyl, pyrazolyl, pyridyl, pyrazyl, pyridazinyl or pyrimidyl substituted by 1 to 3 substituents. Examples of the substituent include halogen, —C₁₋₆ alkyl, —O—C₁₋₆ alkyl, and the like. Specific examples of the “optionally-substituted heteroaryl which is selected from the groupe consisting of furyl, thienyl, oxazolyl, thiazolyl, pyrazolyl, pyridyl, pyrazyl, pyridazinyl and pyrimidyl” include furyl, fluorofuryl, chlorofuryl, bromofuryl, iodofuryl, methylfuryl, ethylfuryl, methoxyfuryl, ethoxyfuryl, thienyl, fluorothienyl, chlorothienyl, bromothienyl, iodothienyl, methylthienyl, ethylthienyl, methoxythienyl, ethoxythienyl, oxazolyl, fluorooxazolyl, chlorooxazolyl, bromooxazolyl, iodooxazolyl, methyloxazolyl, ethyloxazolyl, methoxyoxazolyl, ethoxyoxazolyl, thiazolyl, fluorothiazolyl, chlorothiazolyl, bromothiazolyl, iodothiazolyl, methylthiazolyl, ethylthiazolyl, methoxythiazolyl, ethoxythiazolyl, pyrazolyl, fluoropyrazolyl, chloropyrazolyl, bromopyrazolyl, iodopyrazolyl, methylpyrazolyl, ethylpyrazolyl, methoxypyrazolyl, ethoxypyrazolyl, pyridyl, fluoropyridyl, chloropyridyl, bromopyridyl, iodopyridyl, methylpyridyl, ethylpyridyl, methoxypyridyl, ethoxypyridyl, pyrazyl, fluoropyrazyl, chloropyrazyl, bromopyrazyl, iodopyrazyl, methylpyrazyl, ethylpyrazyl, methoxypyrazyl, ethoxypyrazyl, pyridazinyl, fluoropyridazinyl, chloropyridazinyl, bromopyridazinyl, iodopyridazinyl, methylpyridazinyl, ethylpyridazinyl, methoxypyridazinyl, ethoxypyridazinyl, pyrimidyl, fluoropyrimidyl, chloropyrimidyl, bromopyrimidyl, iodopyrimidyl, methylpyrimidyl, ethylpyrimidyl, methoxypyrimidyl, ethoxypyrimidyl, and the like.

In the present description, “optionally-substituted pyrimidyl” is unsubstituted pyrimidyl or pyrimidyl substituted by 1 to 3 substituents. Examples of the substituent include halogen, —C₁₋₆ alkyl, —O—C₁₋₆ alkyl, and the like. Specific examples of the “optionally-substituted pyrimidyl” include pyrimidyl, fluoropyrimidyl, chloropyrimidyl, bromopyrimidyl, iodopyrimidyl, methylpyrimidyl, ethylpyrimidyl, methoxypyrimidyl, ethoxypyrimidyl, and the like.

In the present description, examples of “alkyl halide” include iodomethane, iodoethane, 1-iodopropane, 2-iodopropane, 1-iodobutane, 2-iodobutane, 1-iodo-2-methylpropane, tert-butyliodide, 1-iodopentane, 2-iodopentane, 1-iodo-2,2-dimethylpropane, 1-iodohexane, 2-iodohexane, 3-iodomethylpentane, and the like.

In the present description, examples of “acid anhydride” include acetic anhydride, propionic anhydride, n-butyric anhydride, isobutyric anhydride, n-valeric anhydride, isovaleric anhydride, pivalic anhydride, n-hexanoic anhydride, heptanoic anhydride, benzoic anhydride, and the like.

In the present description, examples of “acid halide” include benzoyl chloride, acetyl chloride, acetyl bromide, propionyl chloride, n-butyryl chloride, isobutyryl chloride, pentanoyl chloride, isopentanoyl chloride, DL-2-methylbutyryl chloride, pivaloyl chloride, n-hexanoyl chloride, 4-methylpentanoyl chloride, heptanoyl chloride, and the like.

In the present description, examples of “halocarboxylic acid ester” include methyl chloroformate, ethyl chloroformate, propyl chloroformate, isopropyl chloroformate, butyl chloroformate, sec-butyl chloroformate, isobutyl chloroformate, pentyl chloroformate, neopentyl chloroformate, n-hexyl chloroformate, and the like.

In the present description, the “condensing agent” is not particularly limited, and specific examples thereof includes 1-[3-(dimethylamino)propyl]-3-ethylcarbodiimide hydrochloride (WSC·HCl), N,N′-dicyclohexylcarbodiimide (DCC), N,N′-diisopropylcarbodiimide (DIC), N,N′-carbonyldiimidazole (CDI), 4-(4,6-dimethoxy-1,3,5-triazin-2-yl)-4-methyl morpholinium chloride (DMT-MM), benzotriazol-1-yloxytris(dimethylamino)phosphonium hexafluorophosphate (BOP), benzotriazol-1-yloxytripyrrolidinophosphonium hexafluorophosphate (PyBOP), O-(7-azabenzotriazol-1-yl)-1,1,3,3-tetramethyluronium hexafluorophosphate (HATU), (1-cyano-2-ethoxy-2-oxoethylidenaminooxy)dimethylaminomorpholinocarbenium hexafluorophosphate (COMU), and the like, preferably WSC-HCl, HATU and COMU.

In the present description, the “additive” is not particularly limited, and specific examples thereof include 1-hydroxybenzotriazole (HOBt), 1-Hydroxy-7-azabenzotriazole (HOAt), N-Hydroxysuccinimide (HOSu), ethyl (hydroxyimino)cyanoacetate (Oxyma), 4-dimethylaminopyridine (DMAP), triethylamine (TEA), Diisopropylethylamine (DIPEA), N-methylmorpholine, and the like, preferably HOBt, TEA and DIPEA.

Specific examples of the “leaving group” used in the present description include halogen, C₁₋₁₈ alkanesulfonyl, lower alkanesulfonyloxy, arylsulfonyloxy, aralkylsulfonyloxy, perhaloalkanesulfonyloxy, sulfonio, toluenesulfoxy, and the like. A preferable leaving group is halogen.

The “halogen” is fluorine, chlorine, bromine, or iodine.

Examples of the “C₁₋₁₈ alkanesulfonyl” include linear or branched alkanesulfonyl having 1 to 18 carbon atoms, and specific examples thereof include methanesulfonyl, 1-propanesulfonyl, 2-propanesulfonyl, butanesulfonyl, cyclohexanesulfonyl, dodecanesulfonyl, octadecanesulfonyl, and the like.

Examples of the “lower alkanesulfonyloxy” include linear or branched alkanesulfonyloxy having 1 to 6 carbon atoms, and specific examples thereof include methanesulfonyloxy, ethanesulfonyloxy, 1-propanesulfonyloxy, 2-propanesulfonyloxy, 1-butanesulfonyloxy, 3-butanesulfonyloxy, 1-pentanesulfonyloxy, 1-hexanesulfonyloxy, and the like.

Examples of the “arylsulfonyloxy” include phenylsulfonyloxy optionally having 1 to 3 groups selected from the group consisting of linear or branched alkyl having 1 to 6 carbon atoms, linear or branched alkoxy having 1 to 6 carbon atoms, nitro and halogen, as a substituent on the phenyl ring, naphthylsulfonyloxy, and the like. Specific examples of the “phenylsulfonyloxy optionally having substituent(s)” include phenylsulfonyloxy, 4-methylphenylsulfonyloxy, 2-methylphenylsulfonyloxy, 4-nitrophenylsulfonyloxy, 4-methoxyphenylsulfonyloxy, 2-nitrophenylsulfonyloxy, 3-chlorophenylsulfonyloxy, and the like. Specific examples of the “naphthylsulfonyloxy” include α-naphthylsulfonyloxy, β-naphthylsulfonyloxy, and the like.

Examples of the “aralkylsulfonyloxy” include linear or branched alkanesulfonyloxy having 1 to 6 carbon atoms, which is substituted by phenyl optionally having 1 to 3 groups selected from the group consisting of linear or branched alkyl having 1 to 6 carbon atoms, linear or branched alkoxy having 1 to 6 carbon atoms, nitro and halogen, as a substituent on the phenyl ring; and linear or branched alkanesulfonyloxy having 1 to 6 carbon atoms, which is substituted by naphthyl, and the like. Specific examples of the “alkanesulfonyloxy substituted by phenyl” include benzylsulfonyloxy, 2-phenylethylsulfonyloxy, 4-phenylbutylsulfonyloxy, 4-methylbenzylsulfonyloxy, 2-methylbenzylsulfonyloxy, 4-nitrobenzylsulfonyloxy, 4-methoxybenzylsulfonyloxy, 3-chlorobenzylsulfonyloxy, and the like. Specific examples of the “alkanesulfonyloxy substituted by naphthyl” include α-naphthylmethylsulfonyloxy, β-naphthylmethylsulfonyloxy, and the like.

Specific examples of the “perhaloalkanesulfonyloxy” include trifluoromethanesulfonyloxy and the like.

Specific examples of the “sulfonio” include dimethylsulfonio, diethylsulfonio, dipropylsulfonio, di(2-cyanoethyl)sulfonio, di(2-nitroethyl)sulfonio, di-(aminoethyl)sulfonio, di(2-methylaminoethyl)sulfonio, di-(2-dimethylaminoethyl)sulfonio, di-(2-hydroxyethyl)sulfonio, di-(3-hydroxypropyl)sulfonio, di-(2-methoxyethyl)sulfonio, di-(2-carbamoylethyl)sulfonio, di-(2-carbamoylethyl)sulfonio, di-(2-carboxyethyl)sulfonio, di-(2-methoxycarbonylethyl)sulfonio, diphenylsulfonio, and the like.

The “palladium compound” to be used in the present description is not particularly limited, and examples thereof include tetravalent palladium catalysts such as sodium hexachloropalladium (IV) acid tetrahydrate and potassium hexachloropalladium (IV) acid; divalent palladium catalysts such as [1,1′-bis(diphenylphosphino)ferrocene]palladium(II) dichloride dichloromethane adduct (Pd(dppf)Cl₂·CH₂Cl₂), (2-dicyclohexylphosphino-2′,4′,6′-triisopropyl-1,1′-biphenyl) [2-(2′-amino-1,1′-biphenyl)]palladium(II) methanesulfonate (XPhos Pd G3), palladium(II) chloride, palladium(II) bromide, palladium(II) acetate, palladium(II) acetylacetonate, dichlorobis(benzonitrile)palladium(II), dichlorobis(acetonitrile)palladium(II), dichlorobis(triphenylphosphine)palladium(II), dichlorotetraammine palladium(II), dichloro(cycloocta-1,5-diene)palladium(II), and palladium(II) trifluoroacetate; and zerovalent palladium catalysts such as bis(tri-t-butylphosphine)palladium Pd (tBu₃P)₂(0), tris(dibenzylideneacetone)dipalladium(0) (Pd₂(dba)₃), tris(dibenzylideneacetone)dipalladium(0)-chloroform complex, and tetrakis(triphenylphosphine)palladium(0) (Pd(PPh₃)₄). These palladium compounds are used alone or as a mixture of two or more of them.

Examples of the “base” to be used in the present description include an inorganic base, an organic base, and the like.

Examples of the “inorganic base” include alkali metal hydroxides (e.g., lithium hydroxide, sodium hydroxide, and potassium hydroxide), alkaline earth metal hydroxides (e.g., magnesium hydroxide, calcium hydroxide, and barium hydroxide), alkali metal carbonates (e.g., sodium carbonate, potassium carbonate, and cesium carbonate), alkaline earth metal carbonates (e.g., magnesium carbonate, calcium carbonate, and barium carbonate), alkali metal hydrogen carbonates (e.g., sodium hydrogen carbonate and potassium hydrogen carbonate), alkali metal phosphates (e.g., sodium phosphate, potassium phosphate, and cerium phosphate), alkaline earth metal phosphates (e.g., magnesium phosphate and calcium phosphate), alkali metal alkoxides (for example, sodium methoxide, sodium ethoxide, sodium tert-butoxide, and potassium tert-butoxide), alkali metal hydride (for example, sodium hydride and potassium hydride), and the like.

Examples of the “organic base” include trialkylamines (e.g., trimethylamine, triethylamine, and N,N-diisopropylethylamine (DIPEA)), dialkylamine (for example, diethylamine and diisopropylamine), 4-dimethylaminopyridine (DMAP), N-methylmorphiline, picoline, 1,5-diazabicyclo[4.3.0]non-5-ene, 1,4-diazabicyclo[2.2.2]octane, 1,8-diazabicyclo[5.4.0]undec-7-ene, and the like. It is preferably DMAP or TEA.

These bases are used alone or as a mixture of two or more of them.

The “solvent” to be used in the reaction in the present description may be an inert solvent in the reaction, and examples thereof include water, ethers (e.g., dioxane, tetrahydrofuran, diethyl ether, 1,2-dimethoxyethane, diethylene glycol dimethyl ether, and ethylene glycol dimethyl ether), halohydrocarbons (e.g., methylene chloride, chloroform, 1,2-dichloroethane, and carbon tetrachloride), aromatic hydrocarbons (e.g., benzene, toluene, and xylene), lower alcohols (e.g., methanol, ethanol, and isopropanol), and polar solvents (e.g., N,N-dimethylformamide (DMF), N-methylpyrrolidone (NMP), dimethyl sulfoxide (DMSO), hexamethylphosphoric triamide, and acetonitrile). These solvents are used alone or as a mixture of two or more of them.

Each substituent of a compound represented by general formula [I] or [I′] (hereinafter referred to as “compound [I]”) in the present description is described below.

R¹¹ in the compound [I] is hydrogen, halogen, —C₁₋₆ alkyl or —O—C₁₋₆ alkyl, preferably hydrogen, fluorine, chlorine, bromine, iodine, methyl, ethyl, n-propyl, isopropyl, n-butyl, isobutyl, sec-butyl, tert-butyl, n-pentyl, isopentyl, neopentyl, n-hexyl, isohexyl, 3-methylpentyl, —O-methyl, —O— ethyl, —O-n-propyl, —O-isopropyl, —O-n-butyl, —O-isobutyl, —O-sec-butyl, —O-tert-butyl, —O-n-pentyl, —O-isopentyl, —O— neopentyl, —O-n-hexyl, —O-isohexyl or —O-3-methylpentyl, and more preferably hydrogen, chlorine, methyl or —O-methyl.

R¹² in the compound [I] is hydrogen, —C₁₋₆ alkyl, —C₁₋₆ alkyl-O—C₁₋₆ alkyl, —C(═O)—C₁₋₆ alkyl, —C(═O)-aryl or —C(═O)—O—C₁₋₆ alkyl, preferably hydrogen, methyl, ethyl, n-propyl, isopropyl, n-butyl, isobutyl, sec-butyl, tert-butyl, n-pentyl, isopentyl, neopentyl, n-hexyl, isohexyl, 3-methylpentyl, -methyl-O-methyl, -methyl-O-ethyl, -methyl-O-propyl, -ethyl-O-methyl, -ethyl-O-ethyl, -ethyl-O-propyl, -propyl-O-methyl, -propyl-O-ethyl, -propyl-O-propyl, —C(═O)-methyl, —C(═O)-ethyl, —C(═O)-n-propyl, —C(═O)-isopropyl, —C(═O)-n-butyl, —C(═O)-isobutyl, —C(═O)-sec-butyl, —C(═O)-tert-butyl, —C(═O)-n-pentyl, —C(═O)-isopentyl, —C(═O)-neopentyl, —C(═O)-n-hexyl, —C(═O)-isohexyl, —C(═O)-3-methylpentyl, —C(═O)-phenyl, —C(═O)-naphthyl, —C(═O)—O-methyl, —C(═O)—O-ethyl, —C(═O)—O-n-propyl, —C(═O)—O-isopropyl, —C(═O)—O-n-butyl, —C(═O)—O-isobutyl, —C(═O)—O-sec-butyl, —C(═O)—O-tert-butyl, —C(═O)—O-n-pentyl, —C(═O)—O-isopentyl, —C(═O)—O-neopentyl, —C(═O)—O-n-hexyl, —C(═O)—O-isohexyl or —C(═O)—O—3-methylpentyl, and more preferably hydrogen, methyl, -ethyl-O-methyl, —C(═O)-methyl, —C(═O)-phenyl or —C(═O)—O-methyl.

R² in the compound [I] is hydrogen or —C₁₋₆ alkyl, preferably hydrogen, methyl, ethyl, n-propyl, isopropyl, n-butyl, isobutyl, sec-butyl, tert-butyl, n-pentyl, isopentyl, neopentyl, n-hexyl, isohexyl or 3-methylpentyl, and more preferably hydrogen or methyl.

R³ in the compound [I] is halogen, -Qₖ-(C₁₋₆ alkyl)ₘ-Qₚ-R³¹, optionally-substituted phenyl or optionally-substituted heteroaryl which is selected from the groupe consisting of furyl, thienyl, oxazolyl, thiazolyl, pyrazolyl, pyridyl, pyrazyl, pyridazinyl and pyrimidyl, preferably halogen, -Qₖ-(C₁₋₆ alkyl)ₘ-Qₚ-R³¹, optionally-substituted phenyl, furyl, thienyl, oxazolyl, thiazolyl, pyrazolyl, pyridyl, pyrazyl, pyridazinyl or optionally-substituted pyrimidyl, and more preferably fluorine, chlorine, bromine, iodine, —O-methyl, —O-ethyl, —O-propyl, —O-butyl, —O-methyl-O-methyl, —O-ethyl-O-methyl, —O-ethyl-O-ethyl, —O-methyl-cyclopropyl, —O-methyl-cyclobutyl, —O-methyl-cyclopentyl, —O-ethyl-cyclopropyl, —O-ethyl-cyclobutyl, —O-ethyl-cyclopentyl, —S-methyl, —S-ethyl, —S-propyl, -methyl-S-methyl, -methyl-S-ethyl, -ethyl-S-ethyl, —NH-methyl, —NH-ethyl, —C(═O)—O-methyl, —C(═O)—O-ethyl, —C(═O)—O-n-propyl, —C(═O)—O-isopropyl, —C(═O)—O-n-butyl, —C(═O)—O-isobutyl, —C(═O)—O-sec-butyl, —C(═O)—O-tert-butyl, —C(═O)—O-n-pentyl, —C(═O)—O-isopentyl, —C(═O)—O-neopentyl, —C(═O)—O-n-hexyl, —C(═O)—O-isohexyl, —C(═O)—O-3-methylpentyl, phenyl, fluorophenyl, chlorophenyl, bromophenyl, iodophenyl, furyl, thienyl, oxazolyl, thiazolyl, pyrazolyl, pyridyl, pyrazyl, pyrimidyl, fluoropyrimidyl, chloropyrimidyl, bromopyrimidyl, iodopyrimidyl, methylpyrimidyl, ethylpyrimidyl, methoxypyrimidyl, ethoxypyrimidyl or pyridazinyl, and more preferably fluorine, methyl, —O-methyl, —O-ethyl, —O-ethyl-O-methyl, —O-methyl-cyclopropyl, —S-ethyl, -methyl-S-methyl, —NH-ethyl, —C(═O)—O-methyl, phenyl, fluorophenyl, furyl, thienyl, oxazolyl, thiazolyl, pyrazolyl, pyridyl, pyrazyl, pyrimidyl, fluoropyrimidyl, methylpyrimidyl, methoxypyrimidyl or pyridazinyl.

R^{3a} in the compound [I] is —O—C₁₋₆ alkyl, preferably —O— methyl or —O-ethyl.

R^{3b} in the compound [I] is hydrogen or —O—C₁₋₆ alkyl, preferably hydrogen or —O-methyl.

R³¹ in the compound [I] is —C₁₋₆ alkyl or —C₃₋₈ cycloalkyl, preferably methyl, ethyl, n-propyl, isopropyl, n-butyl, isobutyl, sec-butyl, tert-butyl, n-pentyl, isopentyl, neopentyl, n-hexyl, isohexyl, 3-methylpentyl, cyclopropyl, cyclobutyl, cyclopentyl, cyclohexyl, cycloheptyl or cyclooctyl, and more preferably methyl or cyclopropyl.

R⁴ in the compound [I] is hydrogen, halogen, —C₁₋₆ alkyl or —O—C₁₋₆ alkyl, preferably hydrogen, fluorine, chlorine, bromine, iodine, methyl, ethyl, n-propyl, isopropyl, n-butyl, isobutyl, sec-butyl, tert-butyl, n-pentyl, isopentyl, neopentyl, n-hexyl, isohexyl, 3-methylpentyl, —O-methyl, —O— ethyl, —O-propyl or —O-butyl, and more preferably hydrogen, fluorine, methyl or —O-methyl.

Qs in the compound [I] are the same or different and each independently represent oxygen, sulfur, —C(═O)—O— or —NH—.

k, m and p in the compound [I] are the same or different and each independently represent 0 or 1.

n in the compound [I] is 0, 1 or 2, wherein when n is 2, R³s each independently represent the same or different substituent, and preferably 1 or 2.

Vs in the compound [I] are the same or different and each independently represent nitrogen or C—H.

W in the compound [I] is carbon or nitrogen, and preferably carbon.

X in the compound [I] is carbon, nitrogen or N—R¹².

Y in the compound [I] is carbon or nitrogen.

Zs in the compound [I] are the same or different and each independently represent nitrogen or C—H.

Ring A in the compound [I] is aryl or heteroaryl. Examples of the aryl include benzene, naphthalene, anthracene, and the like, preferably benzene. Examples of the heteroaryl include furan, thiophene, oxazole, thiazole, pyrazole, pyridine, pyrimidine, pyridazine, pyrazine, quinoline, isoquinoline, quinazoline, and the like, preferably furan, thiophene, pyridine and quinoline.

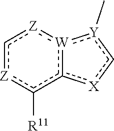

in the compound [I] is, for example,

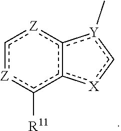

Examples of

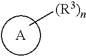

in the compound [I] include ethoxybenzene, methoxyethoxybenzene, cyclopropylmethoxybenzene, ethylsulfanilbenzene, methylsulfanilmethylbenzene, ethylaminobenzene, methyl benzoate, biphenyl, fluorobiphenyl, methoxybiphenyl, pyridylbenzene, pyrimidylbenzene, (fluoropyrimidyl)benzene, (methylpyrimidyl)benzene, (methoxypyrimidyl)benzene, pyrazylbenzene, pyridazinylbenzene, furylbenzene, thienylbenzene, oxazolylbenzene, thiazolylbenzene, pyrazolylbenzene, phenylfuran, ethoxythiophene, phenylthiophene, furylthiophene, thienylthiophene, pyridylthiophene, pyrimidylthiophene, methylquinoline, methoxyquinoline, ethoxypyridine, preferably pyridylbenzene, pyrimidylbenzene, (fluoropyrimidyl)benzene, (methylpyrimidyl)benzene, (methoxypyrimidyl)benzene, phenylthiophene, pyridylthiophene, pyrimidylthiophene, and the like, preferably 2-pyridylbenzene, 2-pyrimidylbenzene, 2-(5-fluoropyrimidyl)benzene, 2-(5-methylpyrimidyl)benzene, 2-(5-methoxypyrimidyl)benzene, 3-phenylthiophene, 3-(2-pyridyl)thiophene, and 3-(2-pyrimidyl)thiophene.

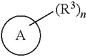

in the compound [I] is, for example,

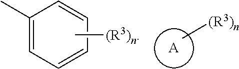

in the compound [I] is, for example,

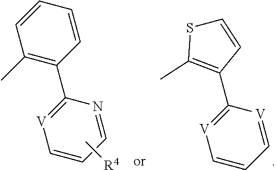

[symbol] in the compound [I] is single bond or double bond.

A preferred compound [I] is, for example, a compound wherein in the general formula [I],

R¹¹ is hydrogen, halogen, —C₁₋₆ alkyl or —O—C₁₋₆ alkyl,

R² is hydrogen,

R³ is phenyl, pyridyl, or pyrimidyl optionally substituted by halogen, —C₁₋₆ alkyl or —O—C₁₋₆ alkyl,

X is N—H,

W and Y are carbon,

Zs are the same or different and each independently represent nitrogen or C—H,

Ring A is benzene or thiophene.

A more preferred compound [I] is, for example, a compound represented by general formula [Ia]:

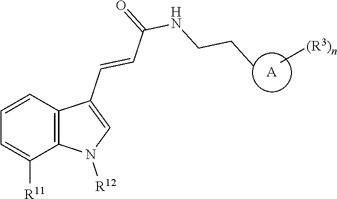

wherein R¹¹ is hydrogen, halogen, —C₁₋₆ alkyl or —O—C₁₋₆ alkyl,
R¹² is hydrogen or —C(═O)—O—C₁₋₆ alkyl,

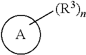

is pyridylbenzene, pyrimidylbenzene (wherein the pyrimidyl is optionally substituted by halogen, —C₁₋₆ alkyl or —O—C₁₋₆ alkyl), phenylthiophene, pyridylthiophene or pyrimidylthiophene,
particulary a compound wherein in the general formula [Ia],
R¹¹ is hydrogen, methyl or —O-methyl,
R¹² is hydrogen or —C(═O)—O-methyl,

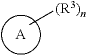

is pyridylbenzene, pyrimidylbenzene, (fluoropyrimidyl)benzene, (methylpyrimidyl)benzene, (methoxypyrimidyl)benzene, phenylthiophene, pyridylthiophene or pyrimidylthiophene.

A further preferred compound [I] is, for example, a compound selected from the group consisting of the following compounds:

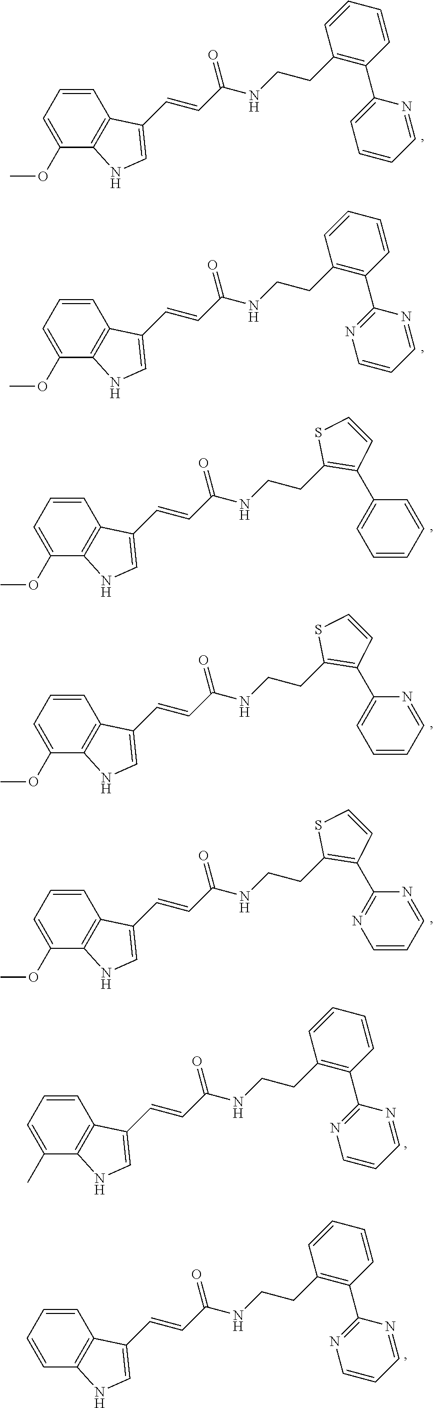

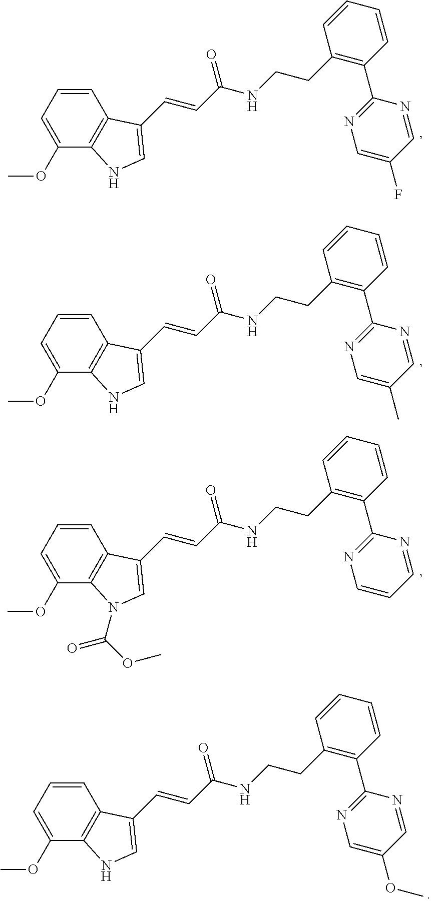

Another preferred compound [I] is, for example, a compound represented by general formula [Ia′]:

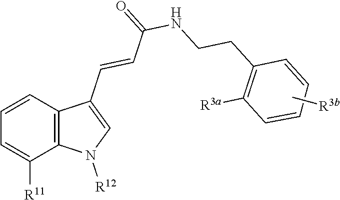

wherein
R^{3a} is —O—C₁₋₆ alkyl;
R^{3b} is hydrogen or —O—C₁₋₆ alkyl;
R¹¹ is —C₁₋₆ alkyl or —O—C₁₋₆ alkyl;
R¹² is hydrogen or —C₁₋₆ alkyl,
particulary a compound wherein in the general formula [Ia′],
R^{3a} is —O-methyl or —O-ethyl,
R^{3b} is hydrogen or —O-methyl,
R¹¹ is methyl or —O-methyl,
R¹² is hydrogen or methyl.

A preferred compound [Ia′] is, for example, a compound selected from the group consisting of the following compounds:

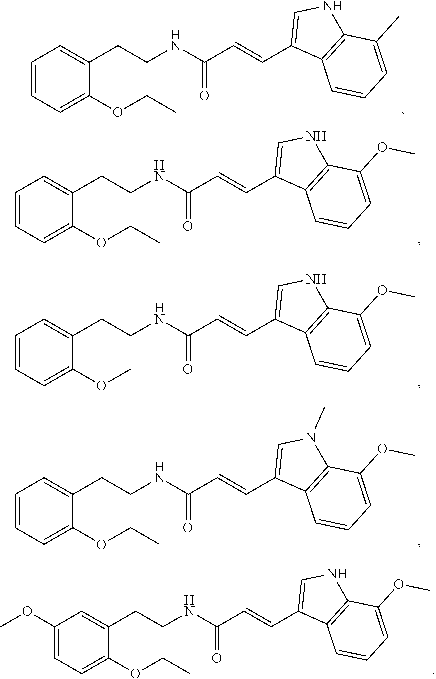

The compound [I] or a salt thereof is useful as a platelet production promoting agent. Therefore, an embodiment of the present invention relates to a platelet production promoting agent comprising the compound [I] or a salt thereof.

The embodiment includes a platelet production promoting agent, which is for use in combination with an aryl hydrocarbon receptor antagonist.

An embodiment of the present invention relates to use of the compound [I] or a salt thereof for promoting platelet production.

The embodiment includes the use wherein the compound [I] or a salt thereof is used in combination with an aryl hydrocarbon receptor antagonist.

An embodiment of the present invention relates to the compound [I] or a salt thereof for use in promoting platelet production.

The embodiment includes the compound [I] or a salt thereof, which is used in combination with an aryl hydrocarbon receptor antagonist.

An embodiment of the present invention relates to a method for promoting platelet production, which comprises culturing platelet progenitor cells in the presence of the compound [I] or a salt thereof.

The embodiment includes the method, which comprises culturing platelet progenitor cells in the copresence of an aryl hydrocarbon receptor antagonist.

An embodiment of the present invention relates to a method for producing platelets, which comprises culturing platelet progenitor cells in the presence of the compound [I] or a salt thereof.

The embodiment includes the method, which comprises culturing platelet progenitor cells in the copresence of an aryl hydrocarbon receptor antagonist.

An embodiment of the present invention relates to a method for culturing platelet progenitor cells to promote platelet production, which comprises culturing platelet progenitor cells in the presence of the compound [I] or a salt thereof.

The embodiment includes the method, which comprises culturing platelet progenitor cells in the copresence of an aryl hydrocarbon receptor antagonist.

In the present description, preferred embodiments and alternatives regarding diverse features of the compound [I] or a salt thereof, use, method, and composition of the present invention can be combined, and unless this is incompatible with the nature thereof, the presentation of the combination of preferred embodiments and alternatives regarding the diverse features is also included.

The method for manufacturing the compound [I] will be described below. The compound [I] can be manufactured according to the method for manufacturing described below. The compound [I] can also be manufactured according to, for example, the method for manufacturing described in WO2019/167973. These methods for manufacturing are examples and the method for manufacturing the compound [I] is not limited thereto.

In the reaction formulae below, in the case of performing alkylation reaction, hydrolysis reaction, amination reaction, esterification reaction, amidation reaction, etherification reaction, nucleophilic substitution reaction, addition reaction, oxidation reaction, reduction reaction, and the like, these reactions are performed according to methods known per se. Examples of such methods include the methods described in Experimental Chemistry (5th edition, The Chemical Society of Japan ed., Maruzen Co., Ltd.); Organic Functional Group Preparations, 2nd edition, Academic Press, Inc. (1989); Comprehensive Organic Transformations, VCH Publishers Inc. (1989); Greene's Protective Groups in Organic Synthesis, 4th edition, (2006) written by P. G. M. Wuts and T. W. Greene; and the like.

### General Synthetic Pathway (1) of the Compound [I]

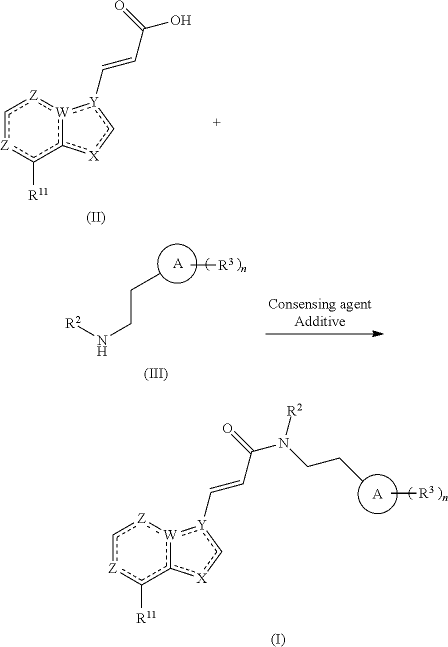

wherein each symbol is as defined above.

The compound [I] can be manufactured by the reaction indicated by the synthetic pathway described above. Specifically, the compound [I] can be manufactured by condensing the compound [II] with the compound [III].

Other reaction conditions (reaction temperature, reaction time, etc.) can be appropriately determined based on a known condensation reaction.

### General Synthetic Pathway (2) of the Compound [I]

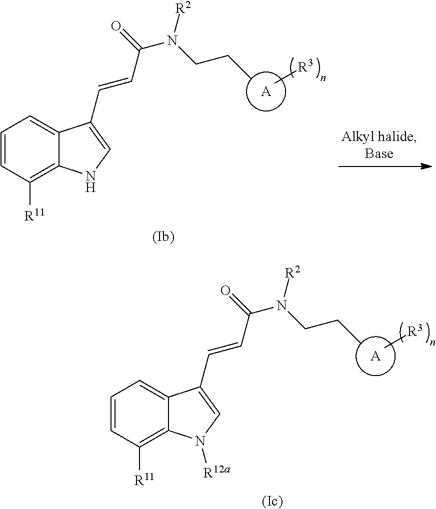

wherein R^{12a} is —C₁₋₆ alkyl, and the other symbols are as defined above.

The compound [Ic] can be manufactured by the reaction indicated by the synthetic pathway described above. Specifically, the compound [Ic] can be manufactured by reacting the compound [Ib] with an alkyl halide.

Other reaction conditions (reaction temperature, reaction time, etc.) can be appropriately determined based on a known condensation reaction.

### General Synthetic Pathway (3) of the Compound [I]

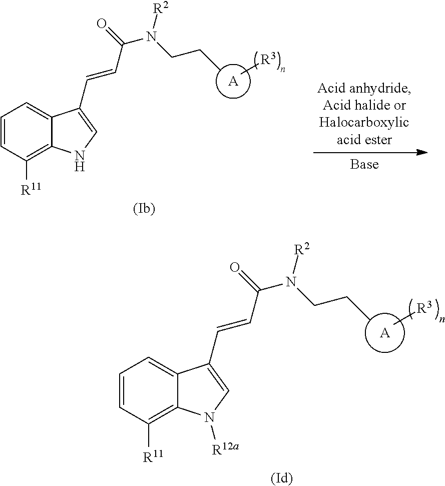

wherein R^{12b} is —C(═O)—C₁₋₆ alkyl C(═O)-aryl or —C(═O)—O—C₁₋₆ alkyl, and the other symbols are as defined above.

The compound [Id] can be manufactured by the reaction indicated by the synthetic pathway described above. Specifically, the compound [Id] can be manufactured by reacting the compound [Ib] with an acid anhydride, an acid halide or a halocarboxylic acid ester.

Other reaction conditions (reaction temperature, reaction time, etc.) can be appropriately determined based on a known condensation reaction.

### General Synthetic Pathway (4) of the Compound [I]

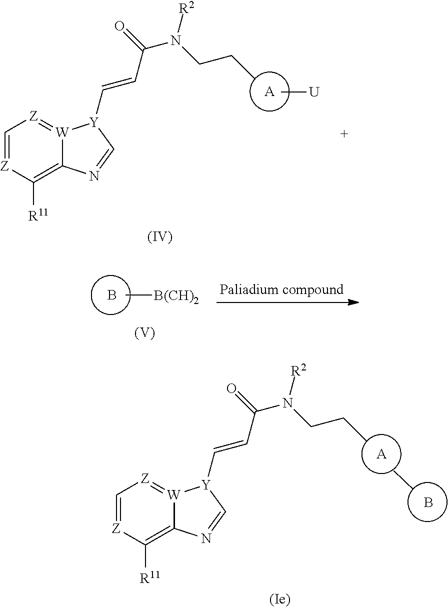

wherein Ring B is optionally-substituted benzene or thiophene, U is a leaving group, and the other symbols are as defined above.

The compound [Ie] of the present invention can be manufactured by the reaction indicated by the synthetic pathway described above. Specifically, the compound [IV] having a leaving group (U) is subjected to coupling reaction with the compound [V] in the presence of a palladium compound, so that the compound [Ie] can be manufactured.

The “boronic acid” or “boronic ester” (the compound [V] in the synthetic pathway) to be used in the present reaction may be separately manufactured, and isolated and purified. For example, bispinacol diborane is subjected to reaction with a halogenated compound as a precursor in the presence of the palladium compound, and the resulting product is subjected to the coupling reaction without isolation and purification.

Other reaction conditions (reaction temperature, reaction time, etc.) can be appropriately determined based on a known coupling reaction.

In each reaction in the above-mentioned equations, the product can be used as a reaction solution or as a crude product thereof in the next reaction. However, the product can be isolated from the reaction mixture in accordance with a conventional method, or easily purified by usual separation means. Examples of the usual separation means include recrystallization, distillation, and chromatography.

The starting material compound, intermediate compound, and objective compound in the above-mentioned steps, and the compound or a salt thereof of the present invention include geometric isomers, stereoisomers, optical isomers, and tautomers. Various isomers can be separated by a general optical resolution method. They can also be manufactured by an appropriate optically active raw material compound.

The compound or a salt thereof of the present invention can be manufactured according to the synthetic methods indicated by the equations described above or methods analogous thereto.

When the specific method of producing the raw material compound used in the manufacturing the compound or a salt thereof of the present invention is not described, the raw material compound may be a commercially available product, or may be a product manufactured according to a method known per se or a method analogous thereto.

The starting material compound and objective compound in the above-mentioned steps can be used in the form of an appropriate salt. Examples of the salt include those similar to the salts exemplified in the following as the salts of the compound of the present invention.

The compound [I] of the present invention includes salt forms thereof including the form of an acid addition salt, or a salt with a base may be formed depending on the kind of the substituent. Examples of the “acid” include an inorganic acid (e.g., hydrochloric acid, hydrobromic acid, nitric acid, sulfuric acid, phosphoric acid, etc.); an organic acid (e.g., methanesulfonic acid, p-toluenesulfonic acid, acetic acid, citric acid, tataric acid, maleic acid, fumaric acid, malic acid, lactic acid, etc.); and the like. Examples of the “base” include an inorganic base (e.g., sodium hydroxide, potassium hydroxide, calcium hydroxide, sodium carbonate, potassium carbonate, sodium hydrogen carbonate, potassium hydrogen carbonate, etc.); an organic base (e.g., methylamine, diethylamine, trimethylamine, triethylamine, ethanolamine, diethanolamine, triethanolamine, ethylenediamine, tris(hydroxymethyl)methylamine, dicyclohexylamine, N,N′-dibenzylethylenediamine, guanidine, pyridine, picoline, choline, etc.); ammonium salts; and the like. In addition, a salt with amino acid such as lysine, arginine, aspartic acid, glutamic acid, and the like may be formed.

The compound [I] of the present invention includes a compound in which one or more atoms are substituted by one or more isotopes. Examples of the isotope include deuterium (²H), tritium (³H), ¹³C, ¹⁵N, ¹⁸O, and the like.

The compound or a salt thereof of the present invention has an activity of promoting platelet production from platelet progenitor cells in vitro.

The method of producing platelets from platelet progenitor cells using the compound or a salt thereof of the present invention will be described below.

Platelets can be produced by culturing platelet progenitor cells (e.g., megakaryocytes or progenitor cells thereof) in the presence of one or two or more kinds of the compound or a salt thereof of the present invention. The concentration of the compound or a salt thereof of the present invention is not particularly limited, and can be appropriately determined by a person skilled in the art depending on a platelet production promoting agent. The concentration thereof is, for example, 1 nM to 100 μM, preferably 10 nM to 100 μM, and further preferably 100 nM to 10 μM, but it may be out of such range as long as a desired effect is exhibited.

Further, the compound or a salt thereof of the present invention can increase the amount of platelets produced from the megakaryocytes. The compound or a salt thereof of the present invention can increase the number of platelets, for example, by 200% or more, preferably 300% or more, further preferably 400% or more, as compared with a control sample, though not limited thereto.

The timing of adding the compound or a salt thereof of the present invention to the medium (or having the compound or a salt thereof present in the medium) is not particularly limited as long as a desired effect is exhibited. For example, the compound or a salt thereof of the present invention is added to megakaryocytes or progenitor cells thereof. The megakaryocytes may be multinucleated or pre-multinucleated, and the multinucleated megakaryocytes includes terminal differentiated form with platelets generation. As described later, in the case of producing immortalized megakaryocytes by forcibly expressing at least one gene selected from the group consisting of a cancer gene, a polycomb gene, and an apoptosis suppressor gene in cells undifferentiated than megakaryocytes and then proceeding with multinucleation of the immortalized megakaryocytes by terminating the forced expression, it is preferable to add the compound or a salt thereof of the present invention to the medium after terminating the forced expression. The compound or a salt thereof of the present invention may be added to the medium at the same time as starting the culturing for platelet production, or 1 day, 2 days, 3 days, 4 days, 5 days, or 6 days after starting the culturing.

Known cells can be used as the megakaryocytes usable in the present invention, and immortalized megakaryocytes can be prepared using the method disclosed in WO 2016/204256, for example.

The origin of megakaryocytes or progenitor cells thereof is not particularly limited as long as they have production ability of platelets, and examples thereof include pluripotent stem cells, in particular, induced pluripotent stem cells (iPS cells) or embryonic stem cells (ES cells). The derivations of iPS cells and ES cells are not particularly limited, and examples thereof include human-derived cells.

The compound or a salt thereof of the present invention can be used as a platelet production promoting agent, in combination with one or two or more aryl hydrocarbon receptor antagonists (AhR antagonist), one or two or more thrombopoietin (TPO) or TPO receptor agonists, one or two or more Rho-associated coiled-coil forming kinase (ROCK) inhibitors, and/or one or two or more disintegrin and metalloprotease (ADAM) inhibitors, and the like.

The compound or a salt thereof of the present invention exhibits more excellent effect of promoting platelet production by culturing platelet progenitor cells in the copresence of an aryl hydrocarbon receptor antagonist.

The aryl hydrocarbon receptor antagonist to be used in combination with the compound or a salt thereof of the present invention is not particularly limited as long as an effect of promoting platelet production is exhibited, but includes, for example, compounds disclosed in WO2020/050409, specifically the following compounds:
4-[2-[[2-benzo[b]thien-3-yl-9-(1-methylethyl)-9H-purin-6-yl]amino]ethyl]phenol (Compound A1)

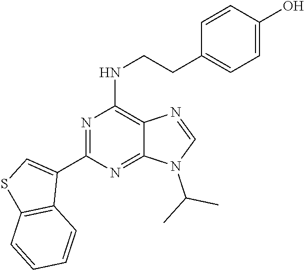 [SMILES: CC(C)n1cnc2c(NCCc3ccc(O)cc3)nc(-c3csc4ccccc34)nc21]

N-[2-(1H-indol-3-yl)ethyl]-9-(1-methylethyl)-2-(5-methyl-3-pyridinyl)-9H-Purin-6-amine (Compound A2)

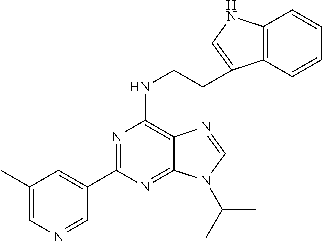 [SMILES: Cc1cncc(-c2nc(NCCc3c[nH]c4ccccc34)c3ncn(C(C)C)c3n2)c1]

4-(2-Methyl-4-pyridinyl)-N-[4-(3-pyridinyl)phenyl]-benzeneacetamide (Compound A3)

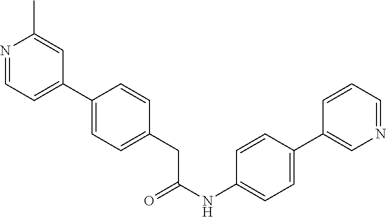 [SMILES: Cc1cc(-c2ccc(CC(=O)Nc3ccc(-c4cccnc4)cc3)cc2)ccn1]

1-Methyl-N-[2-methyl-4-[2-(2-methylphenyl)diazenyl]phenyl]-1H-pyrazole-5-carboxamide (Compound A4)

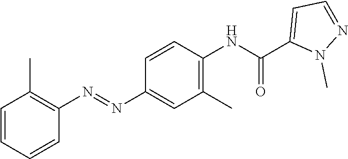 [SMILES: Cc1ccccc1/N=N/c1ccc(NC(=O)c2ccnn2C)c(C)c1]

3-[5-[2-[[2-(5-Fluoropyridin-3-yl)-8,8-dimethyl-7H-purino[8,9-b][1,3]oxazol-4-yl]amino]ethyl]-2-hydroxyphenyl]benzonitrile (Compound A5)

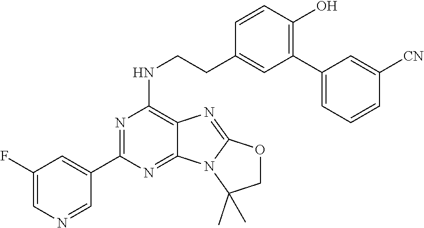 [SMILES: CC1(C)COc2nc3c(NCCc4ccc(O)c(-c5cccc(C#N)c5)c4)nc(-c4cncc(F)c4)nc3n21]

2-(2-fluorophenyl)-4-[2-[[2-(5-fluoropyridin-3-yl)-8,8-dimethyl-7H-purino[8,9-b][1,3]oxazol-4-yl]amino]ethyl]phenol (Compound A6)

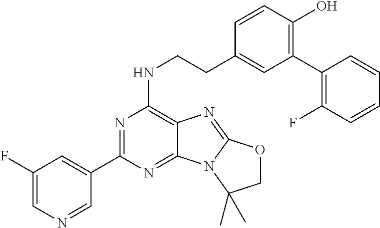 [SMILES: CC1(C)COc2nc3c(NCCc4ccc(O)c(-c5ccccc5F)c4)nc(-c4cncc(F)c4)nc3n21]

2-(5-fluoropyridin-3-yl)-4-[2-[[2-(5-fluoropyridin-3-yl)-8,8-dimethyl-7H-purino[8,9-b][1,3]oxazol-4-yl]amino]ethyl]phenol (Compound A7)

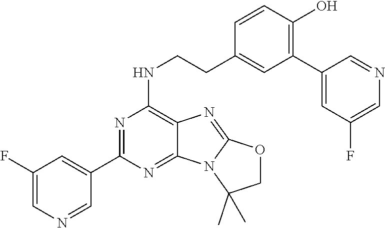 [SMILES: CC1(C)COc2nc3c(NCCc4ccc(O)c(-c5cncc(F)c5)c4)nc(-c4cncc(F)c4)nc3n21]

2-(2-fluorophenyl)-4-[2-[[2-(5-fluoropyridin-3-yl)-8,8-dimethyl-7H-purino[8,9-b][1,3]thiazol-4-yl]amino]ethyl]phenol (Compound A8)

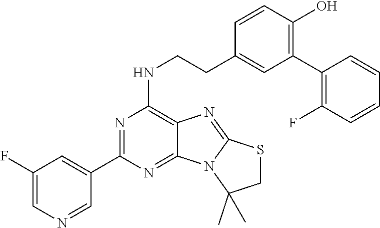 [SMILES: CC1(C)CSc2nc3c(NCCc4ccc(O)c(-c5ccccc5F)c4)nc(-c4cncc(F)c4)nc3n21]

The concentration of the aryl hydrocarbon receptor antagonist is not particularly limited, and can be appropriately determined by a person skilled in the art depending on the compound. The concentration thereof is, for example, in the range of 1.0 nM to 1,000 μM, 10 nM to 100 μM, 100 nM to 100 μM or 100 nM to 10 μM, but it may be out of such range as long as a desired effect is exhibited.

Examples of the ROCK inhibitor include, but are not limited to, Y27632, Y39983, fasudil hydrochloride, ripasudil, SLX-2119, RKI-1447, Azaindole 1, SR-3677, staurosporine, H1152 dihydrochloride, AR-1 2286, INS-117548, and the like. The concentration of the ROCK inhibitor is not particularly limited, and can be appropriately determined by a person skilled in the art depending on the compound. The concentration thereof is, for example, in the range of 1.0 nM to 1.0 mM, 10 nM to 0.1 mM, 100 nM to 0.1 mM, or 100 nM to 0.01 mM, but it may be out of such range as long as a desired effect is exerted.

Thrombopoietin includes thrombopoietin (TPO) and human recombinant thrombopoietin. Examples of the TPO receptor agonist include, but are not limited to, TA-316 and the like. The concentration of the TPO and human recombinant TPO is not particularly limited, and can be appropriately determined by a person skilled in the art. The concentrations of the TPO and the human recombinant TPO are, for example, in the range of 0.5 ng/mL to 5 μg/mL, preferably 5 to 500 ng/mL, and further preferably 50 ng/mL, but it may be out of such range as long as a desired effect is exhibited.

The concentration of the TPO receptor agonist is not particularly limited, and can be appropriately determined by a person skilled in the art depending on the compound. The concentration thereof is, for example, in the range of 0.1 ng/mL to 1 mg/mL, preferably 1 ng/mL to 100 μg/mL, and further preferably 10 ng/mL to 10 μg/mL, but it may be out of such range as long as a desired effect is exhibited.

Examples of the ADAM inhibitor include, but are not limited to, KP-457 and the like. The concentration of the ADAM inhibitor is not particularly limited, and can be appropriately determined by a person skilled in the art depending on the compound. The concentration thereof is, for example, in the range of 1.0 nM to 1.0 mM, preferably 10 nM to 0.1 mM, and further preferably 100 nM to 0.1 mM, but it may be out of such range as long as a desired effect is exhibited.

The compound or a salt thereof of the present invention can be made into a kit in combination with one or two or more aryl hydrocarbon receptor antagonist, one or two or more TPO or TPO receptor agonists, one or two or more ROCK inhibitors, and/or one or two or more ADAM inhibitors, and the like.

The timing of adding the compounds used in combination to the medium (coexisting with the compound or a salt thereof of the present invention in the medium) is not particularly limited as long as a desired effect is exhibited. The compounds used in combination can be added to a medium before, after, or at the same time when the compound or a salt thereof of the present invention is added to the medium. In the case of producing immortalized megakaryocytes by forcibly expressing at least one gene selected from the group consisting of a cancer gene, a polycomb gene, and an apoptosis suppressor gene in cells undifferentiated than megakaryocytes and then proceeding with multinucleation of immortalized megakaryocytes by terminating the forced expression, it is preferable to add the compounds to the medium after termination (including at the same time of termination) of forced expression.

The amount of time for the above-mentioned forced expression is not particularly limited, and can be appropriately determined by a person skilled in the art. Furthermore, the cells may be subcultured following forced expression, and although there are no particular limitations on the amount of time from the final round of subculturing to the day on which forced expression is terminated, that amount of time may be, for example, 1 day, 2 days or 3 days or more.

When the compound or a salt thereof of the present invention is added to the medium after forced expression has been terminated, although the amount of time from the termination of forced expression to the day of addition of the compound or a salt thereof of the present invention to the medium is not particularly limited, culturing may be started in the presence of the compound or a salt thereof of the present invention within, for example, 1 day, 2 days, 3 days, 4 days, 5 days or 6 days. The period of time for culturing cells in the presence of the compound or a salt thereof of the present invention is also not particularly limited. Usually, functional platelets are gradually released starting on about the third day after adding the compound or a salt thereof of the present invention to the medium, and the number of platelets increases with the number of days of culturing. The period of time for culturing cells in the presence of the compound or a salt thereof of the present invention is, for example, 5 to 10 days, but the duration of culturing may be shortened or lengthened. The compound or a salt thereof of the present invention may be added to the medium in one or more additions during the culturing period.

Cell culturing conditions can be those used during ordinary culturing. For example, the temperature can be a temperature of about 35° C. to about 42° C., preferably about 36° C. to about 40° C., or further preferably about 37° C. to about 39° C., and culturing may be carried out in the presence of 5% CO₂ and/or 20% O₂. Culturing may be carried out by static culturing or shake culturing. There are no particular limitations on the shaking speed in the case of shake culturing, and a shaking speed of, for example, 10 rpm to 200 rpm, or preferably 30 rpm to 150 rpm can be used.

When megakaryocytes and/or progenitor cells thereof are brought into contact with the compound or a salt thereof of the present invention and then cultured, matured megakaryocytes are obtained, and platelets are produced from the cytoplasm thereof. Here, maturation of megakaryocytes refers to enabling the megakaryocytes to become multinucleated and release platelets.

There are no particular limitations on the medium used when megakaryocytes are cultured, and a known medium or a medium analogous thereto that is suitable for producing platelets from megakaryocytes can be appropriately used. For example, a medium used to culture animal cells can be prepared as a basal medium. Examples of the basal medium include IMDM medium, Medium 199, Eagle's minimum essential medium (EMEM), αMEM, Dulbecco's modified Eagle's medium (DMEM), Ham's F12 medium, RPMI 1640 medium, Fischer's medium, Neurobasal medium (Life Technologies Corporation), and a mixed medium thereof.

The medium may contain serum or plasma, or may be serum-free. In the case of using serum, fetal bovine serum (FBS) or human serum can be used. The medium can contain one or more substances such as albumin, insulin, transferrin, selenium, fatty acids, trace elements, 2-mercaptoethanol, thiolglycerol, monothioglycerol (MTG), lipid, amino acids (such as L-glutamine), ascorbic acid, heparin, non-essential amino acids, vitamins, growth factors, low molecular weight compounds, antibiotics, antioxidants, pyruvic acid, buffers, inorganic salts or cytokines as necessary. Cytokines are proteins that promote hematopoietic differentiation, and examples thereof include VEGF, TPO, TPO-receptor agonist, SCF, insulin-transferrin-selenite (ITS) supplement, ADAM inhibitors, and the like.

The agents and their amounts to be used, timing of addition to the medium, platelet progenitor cells, their culturing methods and culturing conditions, and the like, described above for the platelet production promoting agent and the platelet production method are similarly applied to other embodiments of the present invention (agents, uses, methods, etc.).

Disclosures of all patent literature and non-patent literature cited in the present description are incorporated in the present description in their entirety by reference.

### EXAMPLES

The present invention is explained in detail in the following by referring to Test Examples, Reference Examples, and Examples, which are not to be construed as limitative, and the invention may be changed within the scope of the present invention.

In the present description, the following abbreviations may be used.

| Abbreviations | Words |
|---|---|
| REX | reference example number |
| EX | example number |
| STR | structural formula |
| RProp | Manufacturing method (numbers indicate that the |
| | compound was manufactured using the corresponding |
| | raw materials in the same way as the reference |
| | example compound having that number as a reference |
| | example number) |
| Prop | Manufacturing method (numbers indicate that the |
| | compound was manufactured using the corresponding |
| | raw materials in the same way as the example |
| | compound having that number as an example number) |
| Data | property data (NMR1: ¹H-NMR (in DMSO-d [symbol] ) |
| | δ (ppm); NMR2: ¹H-NMR (in CDCl [symbol] ) δ (ppm); |
| | MS: mass spectrum) |
| AcOEt | ethyl acetate |
| AcOH | acetic acid |
| AcOK | potassium acetate |
| ACONa | sodium acetate |
| BB [symbol] | boron tribromide |
| n-BuLi | n-butyllithium |
| tBu₃P•HBF₄ | tri-tert-butylphosphonium tetraphenylborate |
| (BPin) [symbol] | bis(pinacolato)diboron |
| CDI | 1,1′-carbonyldiimidazole |
| COMU | (1-cyano-2-ethoxy-2- |
| | oxoethylidenaminooxy)dimethyl- |
| | aminomorpholinocarbenium |
| | hexafluorophosphate |
| m-CPBA | m-chloroperoxybenzoic acid |
| Cs₂CO [symbol] | cesium carbonate |
| DBU | 1,8-diazabicyclo[5.4.0]-7-undecene |
| DCC | dicyclohexylcarbodiimide |
| DCE | 1,2-dichloroethane |
| DCM | dichloromethane |
| DEAD | diethylazodicarboxylate |
| DHP | 3,4-dihydro-2H-pyran |
| DIBAL | diisobutylaluminum hydride |
| DIBOC | di-t-butyl dicarbonate |
| DIPEA | diisopropylethylamine |
| DMA | N,N-dimethylacetamide |
| DMAP | 4-(dimethylamino)pyridine |
| DME | dimethoxyethane |
| DMF | N,N-dimethylformamide |
| DMSO | dimethyl suffoxide |
| DPPA | diphenylphosphoryl azide |
| Et₂O | diethyl ether |
| EtOH | ethanol |
| HATU | O-(7-azabenzotriazol-1-yl)-1,1,3,3-tetramethyloronium |
| | hexafluorophosphate |
| HCl | hydrochloric acid |
| Hexane | n-hexane |
| HOBt | 1-hydroxybenzotriazole |
| IPA | 2-propanol |
| IPE | diisopropyl ether |
| K [symbol] CO₃ | potassium carbonate |
| K [symbol] PO₄ | tripotassium phosphate |
| KHCO [symbol] | potassium hydrogen carbonate |
| KOH | potassium hydroxide |
| KOtBu | potassium tert-butoxide |
| LAH | lithium aluminum hydride |
| LDA | lithium diisopropylamide |
| LHMDS | lithium hexamethyldisilazide |
| LiOH | lithium hydroxide |
| MaCN | acetonitrile |
| MEK | 2-butanone |
| MeOH | methanol |
| NaBH [symbol] | sodium borohydride |
| Na₂CO [symbol] | sodium carbonate |
| NaH | sodium hydride |
| NaHCO₂ | sodium hydrogen carbonate |
| NaOH | sodium hydroxide |
| NaOtBu | sodium t-butoxide |
| NBS | N-bromosuccinimide |
| NCS | N-chlorosuccinimide |
| NHS | N-hydroxysuccinimide |
| NMP | N-methylpyrrolidone |
| Pd/C | palladium-carrying carbon |
| Pd₂(dba) [symbol] | tris(dibenzylideneacetone)dipalladium(0) |
| Pd(tBu [symbol] P) [symbol] | bis(tri-t-butylphosphine)palladium |
| PdCl [symbol] (dppf)DCM | [1,1′-bis(diphenylphosphino)ferrocene]palladium(II) |
| | dichloride dichloromethane adduct |
| Pd(OAc)₂ | palladium(ii) acetate |
| Pd(PPh₃)₄ | tetrakis(triphenylphosphine)palladium(0) |
| P [symbol] /C | palladium-carrying carbon |
| PEG | polyethylene glycol |
| PPTS | pyridinium p-toluenesulfonate |
| TBAF | tetra-n-butylammonium fluoride |
| TCDI | 1,1′-thiocarbonyldiimidazole |
| TEA | triethylamine |
| TFA | trifluoroacetic acid |
| THE | tetrahydrofuran |
| TosMIC | tosylmethyl isocyanide |
| TPP | triphenylphosphine |
| WSC | 3-ethyl-1-(3-dimethylaminopropyl)carbodiimide |
| ZCl | benzyl chloroformate |

[symbol]

indicates data missing or illegible when filed

In the following Examples, “room temperature” generally means about 10° C. to about 35° C. The ratios indicated for mixed solvents are volume mixing ratios, unless otherwise specified. % means wt %, unless otherwise specified.

¹HNMR (proton nuclear magnetic resonance spectrum) was measured by Fourier-transform type NMR (either of Bruker AVANCE III 400 (400 MHz) and Bruker AVANCE III HD (500 MHz)).

Mass spectrum (MS) was measured by LC/MS (ACQUITY UPLC H-Class). As ionization method, ESI method was used. The data indicates actual measured value (found). Generally, molecular ion peaks ([M+H]⁺, [M−H]⁻, etc.) are observed. In the case of a salt, a molecular ion peak or fragment ion peak of free form is generally observed.

In silica gel column chromatography, when denoted as basic, aminopropylsilane-bonded silica gel was used.

The absolute configuration of the compound was determined by known X-ray crystal structure analysis method (e.g., “Basic Course for Chemists 12, X-ray Crystal Structure Analysis” written by Shigeru Ohba and Shigenobu Yano, 1st edition, 1999) or estimated from the empirical rule of Shi asymmetric epoxidation (Waldemar Adam, Rainer T. Fell, Chantu R. Saha-Moller and Cong-Gui Zhao: Tetrahedron: Asymmetry 1998, 9, 397-401; Yuanming Zhu, Yong Tu, Hongwu Yu, Yian Shi: Tetrahedron Lett. 1988, 29, 2437-2440).

### REFERENCE EXAMPLE

### Reference Example 1

### Synthesis of (E)-N-[2-(2-bromophenyl)ethyl]-3-(7-methoxy-1H-indol-3-yl)prop-2-enamide

To a solution of (E)-3-(7-methoxy-1H-indol-3-yl)prop-2-enoic acid (25.0 mg) and 2-bromophenethylamine (19.8 μl) in DCM (2 ml) were added DIPEA (40.2 μl) and COMU (59.1 mg), and the mixture was stirred overnight at room temperature. The reaction mixture was concentrated, and the residue was purified by column chromatography (Hexane/AcOEt) to obtain the object compound (28 mg).

### Reference Example 2

### Synthesis of 2-(2-aminoethyl)-N-ethylaniline dihydrochloride

To a solution of tert-butyl N-[2-(2-aminoethyl)phenyl]-N-ethylcarbamate (180 mg) in EtOH (2 ml) was added 4N HCl/AcOEt (1 ml), and the mixture was stirred at 50° C. for 1.5 hours. The reaction mixture was concentrated, and the residue was washed and dispersed with AcOEt to obtain the object compound (170 mg).

### Reference Example 3

### Synthesis of tert-butyl N-[2-(2-aminoethyl)phenyl]-N-ethylcarbamate

To a solution of tert-butyl N-[2-(2-azidoethyl)phenyl]-N-ethylcarbamate (300 mg) in EtOH (3 ml) was added 10% Pd/C (50 mg), and the mixture was stirred for 3 hours under hydrogen atmosphere at room temperature. The obtained solid was filtered through Celite, and the filtrate was concentrated to obtain the object compound (208 mg).

### Reference Example 4

### Synthesis of tert-butyl N-[2-(2-azidoethyl)phenyl]-N-ethylcarbamate

To a solution of tert-butyl N-[2-(2-azidoethyl)phenyl]carbamate (1.0 g) in DMF (3 ml) were added NaH (0.18 g) and iodoethane (0.37 ml), and the mixture was stirred overnight at room temperature. To the reaction mixture was added water, and the mixture was extracted with AcOEt. The organic layer was washed with saturated saline, dried with anhydrous sodium sulfate, and filtered. The filtrate was concentrated, and the residue was then purified by column chromatography (Hexane/AcOEt) to obtain the object compound (940 mg).

### Reference Example 6

### Synthesis of 2-(3-ethoxythiophene-2-yl)ethaneamine hydrochloride

To a solution of tris(pentafluorophenyl)borane (14.7 mg) in DCM (2 ml) was added a solution of diethylsilane (310 μl) and 2-(3-ethoxythiophene-2-yl)acetonitrile (160 mg) in DCM (1 ml) under nitrogen atmosphere at 0° C. The mixture was stirred at room temperature for 1 hour. The reaction mixture was concentrated, 4N HCl/AcOEt (718 μl) was added to the residue, and the solid precipitate was collected by filtration to obtain the object compound (38 mg).

### Reference Example 7

### Synthesis of 2-(3-ethoxythiophene-2-yl)acetonitrile

To a suspension of KOtBu (524 mg) in DME (4 ml) was added dropwise a solution of TosMIC (502 mg) in DME (3 ml) under nitrogen atmosphere at −50° C., thereto was added dropwise a solution of 3-ethoxythiophene-2-carbaldehyde (365 mg) in DME (3 ml), and the mixture was stirred for 1 hour. The reaction mixture was allowed to warm to room temperature, thereto was added MeOH (10 ml), and the mixture was stirred for 1 hour under reflux with heating. To the reaction mixture was added water, and the mixture was extracted with AcOEt. The organic layer was washed with saturated saline, dried with anhydrous sodium sulfate, and filtered. The filtrate was concentrated under vacuum, and the residue was purified by column chromatography (Hexane/AcOEt) to obtain the object compound (162 mg).

### Reference Example 8

### Synthesis of (E)-N-[2-(2-bromo-5-fluorophenyl)ethyl]-3-(7-methoxy-1H-indol-3-yl)prop-2-enamide

To a solution of (E)-3-(7-methoxy-1H-indol-3-yl)prop-2-enoic acid (25.0 mg) and 2-bromo-5-fluorophenethylamine (30.1 mg) in DCM (2 ml) were added DIPEA (40.2 μl) and HATU (52.5 mg), and the mixture was stirred at room temperature for 1 hour. The reaction mixture was concentrated, and the residue was purified by column chromatography (Hexane/AcOEt) to obtain the object compound (43 mg).

### Reference Example 13

### Synthesis of 2-(2-pyrimidin-2-ylphenyl)ethaneamine hydrochloride

To a solution of tert-butyl N-[2-(2-bromophenyl)ethyl]carbamate (200 mg) in toluene (4 ml) were added 2-tributylstannylpyrimidine (232 μl) and Pd(PPh₃)₄(77.0 mg) under argon atmosphere, and the mixture was stirred overnight under reflux with heating. The reaction mixture was concentrated, and the residue was purified by column chromatography (Hexane/AcOEt). To a solution of the purified product in EtOH (1 ml) was added 4N HCl/AcOEt (0.5 ml), and the mixture was stirred at 50° C. for 1.5 hours. The reaction mixture was concentrated to obtain the object compound (76.0 mg).

### Reference Example 14

### Synthesis of (E)-N-[2-(3-bromothiophene-2-yl)ethyl]-3-(7-methoxy-1H-indol-3-yl)prop-2-enamide

To a suspension solution of LAH (0.084 g) in THF (4 ml) was added dropwise a solution of 3-bromo-2-[(E)-2-nitroethenyl]thiophene (400 mg) in THF (3 ml) at 0° C. under nitrogen atmosphere, and the mixture was stirred at room temperature for 2 hours. To the reaction mixture were added water (0.15 ml), 15% NaOH aqueous solution (0.15 ml) and water (0.45 ml), the mixture was filtered through Celite, and the filtrate was concentrated. To a solution of the residue in DCM (1 ml) were added (E)-3-(7-methoxy-1H-indol-3-yl)prop-2-enoic acid (40.0 mg), DIPEA (0.048 ml) and HATU (91.0 mg), and the mixture was stirred overnight at room temperature. The reaction mixture was purified by column chromatography (Hexane/AcOEt) to obtain the object compound (0.032 g).

### Reference Example 15

### Synthesis of 2-(3-thiophene-2-ylthiophene-2-yl)ethaneamine hydrochloride

A mixture of tert-butyl N-[2-(3-bromothiophene-2-yl)ethyl]carbamate (57.0 mg), 2-thiopheneboronic acid (40.5 mg), PdCl₂(dppf)DCM (7.6 mg), K₃PO₄ (79.0 mg) and 1,4-dioxane/water (4/1) (1 ml) was stirred under nitrogen atmosphere at 90° C. for 2 hours. The reaction mixture was purified by column chromatography (Hexane/AcOEt). To a solution of the purified product in EtOH (0.5 ml) was added 4N HCl/AcOEt (0.5 ml), and the mixture was stirred overnight at room temperature. The reaction mixture was concentrated to obtain the object compound (38.2 mg).

### Reference Example 19

### Synthesis of 2-(2-pyrimidin-4-ylphenyl)ethaneamine hydrochloride

A mixture of tert-butyl N-[2-[2-(4,4,5,5-tetramethyl-1,3,2-dioxaborolan-2-yl)phenyl]ethyl]carbamate (150 mg), 4-chloropyrimidine hydrochloride (98.0 mg), PdCl₂(dppf)DCM (35.3 mg), K₃PO₄ (183 mg) and DME/water (4/1) (2 ml) was stirred overnight under reflux with heating under nitrogen atmosphere. The reaction mixture was concentrated, and the residue was purified by column chromatography (Hexane/AcOEt). To a solution of the purified product in EtOH (1 ml) was added 4N HCl/AcOEt (0.5 ml), and the mixture was stirred at 50° C. for 1.5 hours. The reaction mixture was concentrated to obtain the object compound (55.0 mg).

### Reference Example 20

### Synthesis of (E)-3-(7-methoxy-1H-pyrrolo[2,3-c]pyridine-3-yl)prop-2-enoic acid

To a solution of 7-methoxy-1H-pyrrolo[2,3-c]pyridine (420 mg) in AcOH (3 ml) was added hexamethylenetetramine (265 mg), and the mixture was stirred at 100° C. for 6 hours. To the reaction mixture was added saturated NaHCO₃ aqueous solution, and the mixture was extracted with AcOEt. The organic layer was washed with saturated saline, dried with anhydrous sodium sulfate, and filtered. The filtrate was concentrated, and the residue was suspended in DCM (3 ml). To the mixture were added DIBOC (439 μl) and DMAP (23.1 mg), and the mixture was stirred for 30 minutes. The reaction mixture was concentrated, and the residue was purified by column chromatography (Hexane/AcOEt).

To a solution of ethyl diethyl phosphonoacetate (113 μl) in THF (3 ml) was added NaH (22.7 mg), and the mixture was stirred for 30 minutes. To the reaction mixture was added dropwise a solution of the above purified product (104 mg) in THF (2 ml), and the mixture was stirred at room temperature for 1 hour. To the reaction mixture was added water, and the mixture was extracted with AcOEt. The organic layer was washed with saturated saline, dried with anhydrous sodium sulfate, and filtered. The filtrate was concentrated, and the residue was purified by column chromatography (Hexane/AcOEt).

To a solution of the purified product (104 mg) in THF-MeOH-water (1:1:1) (6 ml) was added 5N NaOH aqueous solution (240 μl), and the mixture was stirred overnight under reflux with heating. The reaction mixture was concentrated, and 1N HCl aqueous solution was added to the residue to neutralize it. The solid precipitate was collected by filtration to obtain the object compound (48.0 mg).

### Reference Example 22

### Synthesis of (E)-3-(4-methoxyindol-1-yl)prop-2-enoic acid

To a solution of 4-methoxyindole (300 mg) in DMF (3 ml) were added Cs₂CO₃(996 mg) and ethyl propiolate (248 μl), and the mixture was stirred at room temperature for 1 hour. To the reaction mixture was added water, and the mixture was extracted with AcOEt. The organic layer was washed with saturated saline, dried with anhydrous sodium sulfate, and filtered. The filtrate was concentrated, and the residue was purified by column chromatography (Hexane/AcOEt). To a solution of the purified product in THF-tert-butanol-water (1:1:0.5) (9 ml) was added 5N NaOH aqueous solution (636 μl), and the mixture was stirred for 3 hours under reflux with heating. The reaction mixture was concentrated, and 1N HCl aqueous solution was added to the residue. The solid precipitate was collected by filtration to obtain the object compound (212 mg)

### Reference Example 23

### Synthesis of (E)-3-(8-methoxyimidazo[1,2-a]pyridine-3-yl)prop-2-enoic acid

To a solution of ethyl diethyl phosphonoacetate (378 μl) in THF (5 ml) was added NaH (76.0 mg), and the mixture was stirred for 1 hour. To the reaction mixture was added dropwise a solution of 8-methoxyimidazo[1,2-a]pyridine-3-carbaldehyde (280 mg) in THF (10 ml), and the mixture was stirred at room temperature for 1 hour. To the reaction mixture was added water, and the mixture was extracted with AcOEt. The organic layer was washed with saturated saline, dried with anhydrous sodium sulfate, and filtered. The filtrate was concentrated, and the residue was washed with IPE. To a solution of the purified product in THF-MeOH-water (1:1:1) (6 ml) was added 5N NaOH aqueous solution (804 μl), and the mixture was stirred overnight under reflux with heating. The reaction mixture was concentrated, and 5N HCl aqueous solution was added to the residue to make it weak acidity. The solid precipitate was collected by filtration to obtain the object compound (212 mg).

### Reference Example 26

### Synthesis of 2-[2-(5-fluoropyrimidin-2-yl)phenyl]ethanamine hydrochloride

A mixture of tert-butyl N-[2-[2-(4,4,5,5-tetramethyl-1,3,2-dioxaborolan-2-yl)phenyl]ethyl]carbamate (222 mg), 2-chloro-5-fluoropyrimidine (118 μl), Pd(tBu₃P)₂ (16.3 mg), K₃PO₄ (271 mg) and 1,4-dioxane/water (4/1) (2.5 ml) was stirred under nitrogen atmosphere at 90° C. for 7 hours. The reaction mixture was concentrated, and the residue was purified by column chromatography (Hexane/AcOEt). To a solution of the purified product in EtOH (1 ml) was added 4N HCl/AcOEt (0.5 ml), and the mixture was stirred at 50° C. for 1.5 hours. The reaction mixture was concentrated to obtain the object compound (128 mg)

The compounds of Reference Examples 5, 9 to 12, 16 to 18, 21, 24, 25, 27 and 28 were manufactured in the same manner as in Reference Examples 1 to 4, 6 to 8, 13 to 15, 19, 20, 22, 23 and 26. Structural formulae and physicochemical data of the compounds of Reference Examples 1 to 28 are shown in Tables 1-1 to 1-5.

TABLE 1-1

| REX | STR | RProp | Data |
|---|---|---|---|
| 1 | 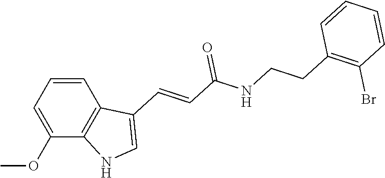 | 1 | NMR2(500 MHz): 8.65 (1H, s), 7.86 (1H, d, J = 15 5 Hz), 7.56 (1H, dd, J = 7.8, 1.1 Hz), 7.45 (1H, d, J = 8.0 Hz), 7.41 (1H, d, J = 2.7 Hz), 7.31-7 21 (2H, m), 7.12- 7.07 (2H, m), 6.71 (1H, d, J = 7.7 Hz), 6.36 (1H, d. J = 15.6 Hz), 5.64 (1H, t, J = 6.0 Hz), 3.96 (3H, s), 3.73-3.65 (2H, m), 3.06 (2H, t. J = 7.0 Hz). |
| 2 | 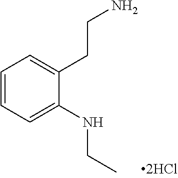 | 2 | NMR2(500 MHz): 11.13 (2H, s), 8.30 (3H, s), 7.68 (1H, d, J = 7.9 Hz), 7.44-7.35 (2H. m), 7.32-7.26 (1H, m), 3.50-3.26 (6H, m), 1.48-1.38 (3H, m). |
| 3 | 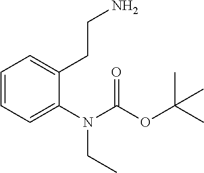 | 3 | NMR2(500 MHz): 7.29-7.19 (3H, m), 7.06-7.02 (1H, m), 3.94-3.72 (1H, m), 3.30-3.19 (1H, m), 2.99-2.93 (2H, m), 2.79-2.58 (2H, m), 1.60-1.05 (12H, m), MS m/z 265.32 (M + 1). |
| 4 | 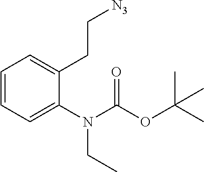 | 4 | NMR2(500 MHz): 7.34-7.01 (4H, m), 3.93-3.64 (1H, m), 3.64-3.26 (3H, m), 2.92-2.73 (2H, m), 1.57-1.10 (12H, m). |
| 5 | 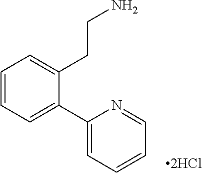 | 13 | MS m/z 199.20 (M + 1). |
| 6 | 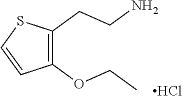 | 6 | NMR1(500 MHz): 8.00 (3H, s), 7.32 (1H, d, J = 5.5 Hz), 6.99 (1H, d, J = 5.5 Hz), 4.04 (2H, q, J = 7.0 Hz), 2.93 (4H, s), 1.28 (3H, t, J = 7.0 Hz). |

TABLE 1-2

| REX | STR | RProp | Data |
|---|---|---|---|
| 7 | 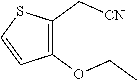 | 7 | NMR2(500 MHz): 7.15 (1H, d, J = 5.5 Hz), 6.80 (1H, d, J = 5.6 Hz), 4.09 (2H, q, J = 7.0 Hz), 3.76 (2H, s), 1.38 (3H, t, J = 7.0 Hz). |
| 8 | 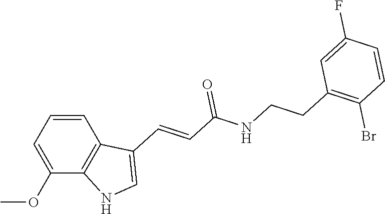 | 8 | NMR2(500 MHz): 8.63 (1H, s), 7.86 (1H, d, J = 15.6 Hz), 7.51 (1H, dd, J = 8.7, 5.3 Hz), 7.46 (1H, d, J = 8.0 Hz), 7.42 (1H, d, J = 2.8 Hz), 7.14 (1H, t, J = 7.9 Hz), 7.02 (1H, dd, J = 9.2, 3.0 Hz), 6.87-6.81 (1H, m), 8.72 (1H, d, J = 7.7 Hz), 6.37 (1H, d, J = 15.5 Hz), 5.67-5.61 (1H, m), 3.97 (3H, s), 3.71-3.64 (2H, m), 3.04 (2H, t, J = 7.0 Hz). |
| 9 | 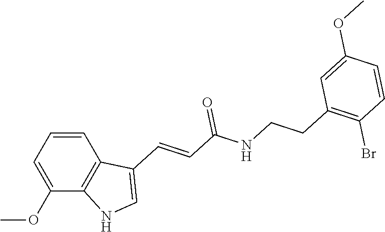 | 8 | NMR2(500 MHz): 5.67 (1H, s), 7.86 (1H, d, J = 15.5 Hz), 7.48-7.39 (3H, m), 7.14 (1H, t, J = 7.9 Hz), 6.82 (1H, d, J = 3.0 Hz), 6.71 (1H, d, J = 7.2 Hz), 6.67 (1H, dd, J = 8.8, 3.0 Hz), 6.36 (1H, d, J = 15.6 Hz), 5.67 (1H, t, J = 6.0 Hz), 3.96 (3H, s), 3.75 (3H, s), 3.71-3.64 (2H, m), 3.02 (2H, t, J = 7.0 Hz). |
| 10 | 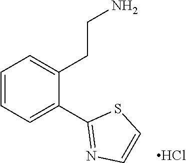 | 13 | MS m/z 205.19 (M + 1). |
| 11 | 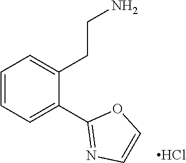 | 13 | MS m/z 189.21 (M + 1). |

TABLE 1-3

| REX | STR | RProp | Data |
|---|---|---|---|
| 12 | 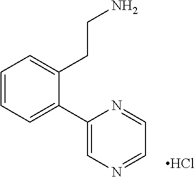 | 13 | MS m/z 200.26 (M + 1). |
| 13 | 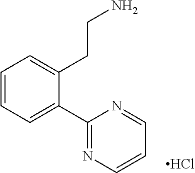 | 13 | NMR1(500 MHz): 8.97 (2H, d, J = 4.9 Hz), 8.11 (3H, s), 7.86 (1H, d, J = 7.6 Hz), 7.65- 7.45 (2H, m), 7.45-7.39 (2H, m), 3.17- 3.03 (4H, m). MS m/z 200.22 (M + 1). |
| 14 | 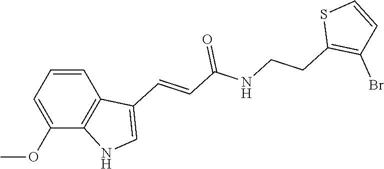 | 14 | NM2(500 MHz): 8.67 (1H, s), 7.85 (1H, d, J = 15.6 Hz), 7.49-7.40 (2H, m), 7.20- 7.12 (2H, m), 6.96 (1H, d, J = 4.9 Hz), 6.72 (1H, d, J = 7.8 Hz), 6.37 (1H, d, J = 15.6 Hz), 5.72 (1H, t, J = 6.1 Hz), 3.96 (3H, s), 3.71-3.84 (2H, m), 3.11 (2H, t, J = 6.7 Hz). |
| 15 | 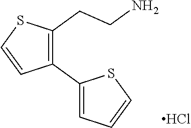 | 15 | NMR1(500 MHz): 8.00 (3H, s), 7.61 (1H, dd, J = 5.1, 1.2 Hz), 7.52 (1H, d, J = 5.2 Hz), 2.28 (1H, dd, J = 3.6, 1.2 Hz), 7.22 (1H, d, J = 5.2 Hz), 7.17 (1H, dd, J = 5.1, 3.6 Hz), 3.30-3.23 (2H, m), 3.09-3.01 (2H, m). |
| 16 | 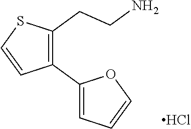 | 15 | NMR1(500 MHz): 8.01 (3H, s), 7.73 (1H, d, J = 1.8 Hz), 7.49 (1H, d, J = 5.3 Hz), 7.33 (1H, d, J = 5.3 Hz), 6.78 (1H, d, J = 3.3 Hz), 6.62 (1H, dd, J = 3.4, 1.8 Hz), 3.35-3.28 (2H, m), 3.09-3.01 (2H, m). |
| 17 | 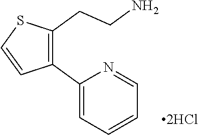 | 13 | NMR1(500 MHz): 8.73 (1H, d, J = 5.0 Hz), 8.16 (3H, s), 8.12-8.05 (1H, m), 7.84 (1H, d, J = 8.0 Hz), 7.55 (1H, d, J = 5.3 Hz), 7.55-7.51 (1H, m), 7.46 (1H, d, J = 5.3 Hz), 3.41-3.36 (2H, m), 3.15-3.06 (2H, m). |

TABLE 1-4

| REX | STR | RProp | Data |
|---|---|---|---|
| 18 | 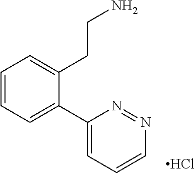 | 19 | MS m/z 200.19 (M + 1). |
| 19 | 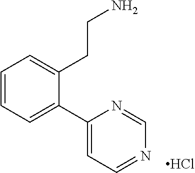 | 19 | MS m/z 200.19 (M + 1). |
| 20 | 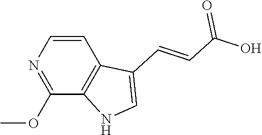 | 20 | NMR1(500 MHz): 12.51-11.50 (2H, m), 7.97 (1H, s), 7.81-7.75 (2H, m), 7.44 (1H, d, J = 5.7 Hz), 6.30 (1H, d, J = 16.0 Hz), 4.03 (3H, s). |
| 21 | 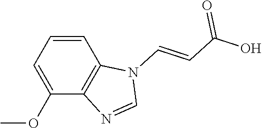 | 22 | NMR1(400 MHz): 12.51 (1H, s), 8.71 (1H, s), 8.25 (1H, d, J = 14.4 Hz), 7.50 (1H, d, J = 8.1 Hz), 7.31 (1H, t. J = 8.1 Hz), 6.89 (1H, d, J = 8.0 Hz), 6.50 (1H, d, J = 14.4 Hz), 3.98 (3H, s). |
| 22 | 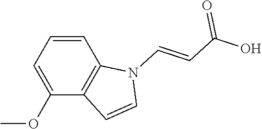 | 22 | NMR1(400 MHz): 12.15 (1H, brs), 8.23 (1H, d, J = 14.0 Hz), 7.85 (1H, d, J = 3.6 Hz), 7.41 (1H, d, J = 8.3 Hz), 7.23 (1H, t, J = 8.1 Hz), 6.79-6.71 (2H, m), 6.24 (1H, d, J = 14.0 Hz), 3.89 (3H, s). |
| 23 | 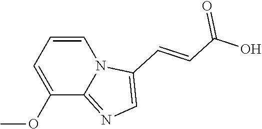 | 23 | NMR1(500 MHz): 12.38 (1H, s), 8.43 (1H, d, J = 6.8 Hz), 8.18 (1H, s), 7.91 (1H, d, J = 15.9 Hz), 6.98 (1H, t, J = 7.2 Hz), 6.84 (1H, d, J = 7.7 Hz), 8:52 (1H, d, J = 15.9 Hz), 3.95 (3H, s). |
| 24 | 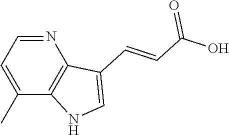 | 20 | NMR1(400 MHz): 12.15-11.76 (2H, m), 8.33 (1H, d, J = 4.7 Hz), 8.05 (1H, d, J = 3.1 Hz), 7.76 (1H, d, J = 15.8 Hz), 7.11 (1H, d, J = 15.6 Hz), 7.04 (1H, d, J = 4.8 Hz), 2.50 (3H, s). |

TABLE 1-5

| REX | STR | RProp | Date |
|---|---|---|---|
| 25 | 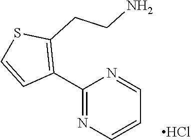 | 13 | NMR1(500 MHz): 8.89 (2H, d, J = 4.8 Hz), 7.96 (3H, s), 7.78 (1H, d, J = 5.3 Hz), 7.61 (1H, d, J = 5.3 Hz), 7.41 (1H, t, J = 4.9 Hz), 3.62-3.55 (2H, m), 3.19-3.11 (2H, m). |
| 26 | 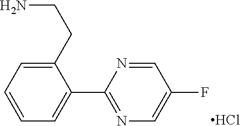 | 26 | NMR1(500 MHz): 9.05 (2H, s), 8.12 (3H, s), 7.82 (1H, d, J = 7.9 Hz), 7.52-7.38 (3H, m), 3.14-2.90 (4H, m). |
| 27 | 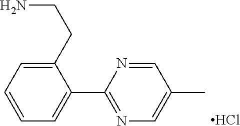 | 26 | NMR1(500 MHz): 8. [symbol] 1 (2H, s), 8.13 (3H, s), 7.85-7.80 (1H, m), 7. [symbol] 0-7.37 (3H, m), 3.17-3.02 (4H, m), 2.35 (3H, s). |
| 28 | 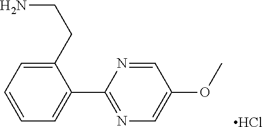 | 26 | NMR 1(500 MHz): 8.70 (2H, s), 7.99 (3H, s), 7.83-7.75 (1H, m), 7.45-7.36 (3H, m), 3.88 (3H, s), 3.15-3.02 (4H, m). |

[symbol]

indicates data missing or illegible when filed

### EXAMPLE

### Example 10

### Synthesis of (E)-N-[2-[2-(cyclopropylmethoxy)phenyl]ethyl]-3-(7-methoxy-1-methylindol-3-yl)-N-methylprop-2-enamide

To a solution of (E)-N-[2-[2-(cyclopropylmethoxy)phenyl]ethyl]-3-(7-methoxy-1H-indol-3-yl)-N-methylprop-2-enamide (25.0 mg) in DMF (1 ml) were added iodomethane (5.80 μl) and Cs₂CO₃ (40.3 mg), and the mixture was stirred at room temperature for 5 hours. To the reaction mixture was added water, and the mixture was extracted with AcOEt. The organic layer was washed with saturated saline, dried with anhydrous sodium sulfate, and filtered. The filtrate was concentrated, and the residue was purified by column chromatography (Hexane/AcOEt) to obtain the object compound (23.0 mg).

### Example 17

### Synthesis of (E)-3-(7-methoxy-1H-indol-3-yl)-N-[2-(2-pyrimidin-2-ylphenyl)ethyl]prop-2-enamide

To a solution of (E)-3-(7-methoxy-1H-indol-3-yl)prop-2-enoic acid (30.0 mg) and 2-(2-pyrimidin-2-ylphenyl)ethanamine hydrochloride (71.6 mg) in DCM (3 ml) were added DIPEA (96.0 μl) and COMU (71.0 mg), and the mixture was stirred at room temperature for 2 hours. To the reaction mixture was added saturated NaHCO₃ aqueous solution, and the mixture was extracted with AcOEt. The organic layer was washed with saturated saline, dried with anhydrous sodium sulfate, and filtered. The filtrate was concentrated, and the residue was purified by column chromatography (Hexane/AcOEt) to obtain the object compound (40 mg).

### Example 19

### Synthesis of (E)-3-(7-methoxy-1H-indol-3-yl)-N-[2-(2-thiophene-2-ylphenyl)ethyl]prop-2-enamide

To a mixture of (E)-N-[2-(2-bromophenyl)ethyl]-3-(7-methoxy-1H-indol-3-yl)prop-2-enamide (30.0 mg), 2-thiopheneboronic acid (12.5 mg), PdCl₂(dppf)DCM (3.1 mg), K₃PO₄ (31.9 mg) and 1,4-dioxane/water (4/1) (1 ml) was stirred under nitrogen atmosphere at 90° C. for 6 hours. The reaction mixture was purified by column chromatography (Hexane/AcOEt) to obtain the object compound (24.7 mg).

### Example 24

### Synthesis of (E)-3-(1-acetyl-7-methoxyindol-3-yl)-N-[2-(2-phenylphenyl)ethyl]prop-2-enamide

To a solution of (E)-3-(7-methoxy-1H-indol-3-yl)-N-[2-(2-phenylphenyl)ethyl]prop-2-enamide (25.0 mg) in DCE (0.6 ml) were added TEA (0.050 ml), DMAP (7.2 mg) and acetic anhydride (0.011 ml), and the mixture was stirred overnight at room temperature. The reaction mixture was purified by column chromatography (Hexane/AcOEt). To a solution of the purified product in DCE (0.6 ml) were added TEA (0.050 ml), DMAP (3.0 mg) and acetic anhydride (0.011 ml), and the mixture was stirred at room temperature for 1 hour. The reaction mixture was purified by column chromatography (Hexane/AcOEt) to obtain the object compound (20.2 mg).

### Example 25

### Synthesis of (E)-3-(1-benzoyl-7-methoxyindol-3-yl)-N-[2-(2-phenylphenyl)ethyl]prop-2-enamide

To a solution of (E)-3-(7-methoxy-1H-indol-3-yl)-N-[2-(2-phenylphenyl)ethyl]prop-2-enamide (19.7 mg) in DCE (0.6 ml) were added TEA (0.039 ml), DMAP (5.7 mg) and benzoyl chloride (0.011 ml), and the mixture was stirred at room temperature for 3 hours. The reaction mixture was purified by column chromatography (Hexane/AcOEt) to obtain the object compound (22.2 mg).

### Example 29

### Synthesis of (E)-N-[2-(2-ethoxypyridine-3-yl)ethyl]-3-(7-methoxy-1H-indol-3-yl)prop-2-enamide

To a mixture of 2-(2-ethoxypyridine-3-yl)acetonitrile (140 mg), NaBH₄ (140 mg) and THF (3 ml) was added TFA (0.28 ml) at 0° C., and the mixture was stirred at room temperature for 1 hour. To the reaction mixture were added water and saturated NaHCO₃ aqueous solution, and the mixture was exracted with AcOEt. The organic layer was concentrated. To a solution of the residue in DCM (1 ml) were added (E)-3-(7-methoxy-1H-indol-3-yl)prop-2-enoic acid (30.0 mg), DIPEA (0.036 ml) and HATU (68.3 mg), and the mixture was stirred at room temperature for 1 hour. The reaction mixture was purified by column chromatography (Hexane/AcOEt) to obtain the object compound (10.8 mg).

### Example 45

### Synthesis of (E)-3-(7-methoxy-1H-indol-3-yl)-N-[2-(3-pyrimidin-2-ylthiophen-2-yl)ethyl]prop-2-enamide

To a solution of 2-(3-pyrimidin-2-ylthiophene-2-yl)ethanamine hydrochloride (16.2 mg) in DCM (0.6 ml) were added DIPEA (82.0 μl), (E)-3-(7-methoxy-1H-indol-3-yl)prop-2-enoic acid (15.0 mg) and HATU (33.1 mg), and the mixture was stirred overnight at room temperature. The reaction mixture was purified by column chromatography (Hexane/AcOEt) to obtain the object compound (11.8 mg).

### Example 47

### Synthesis of (E)-3-(1H-indol-3-yl)-N-[2-(2-pyrimidin-2-ylphenyl)ethyl]prop-2-enamide

To a solution of 2-(2-pyrimidin-2-ylphenyl)ethanamine hydrochloride (45.0 mg) in DCM (0.6 ml) were added DIPEA (128 μl), (E)-3-(1H-indol-3-yl)prop-2-enoic acid (27.5 mg) and HATU (72.6 mg), and the mixture was stirred overnight at room temperature. The reaction mixture was purified by column chromatography (Hexane/AcOEt/MeOH) to obtain the object compound (36.1 mg).

The compounds of Examples 1 to 9, 11 to 16, 18, 20 to 23, 26 to 28, 30 to 44, 46 and 48 to 59 were manufactured in the same manner as in Examples 10, 17, 19, 24, 25, 29, 45 and 47. Structural formulae and physicochemical data of the compounds of Examples 1 to 59 are shown in Tables 2-1 to 2-13.

TABLE 2-1

| EX | STR | Prop | Data |
|---|---|---|---|
| 1 | 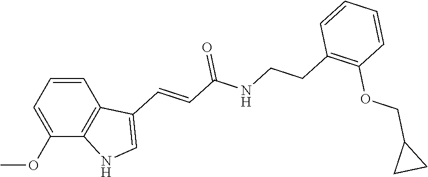 | 17 | NMR2(500 MHz); 8.72 (1H, s), 7.82 (1H, d, J = 15.6 Hz), 7.43 (1H, d, J = 8. 1 Hz), 7.38 (1H, d, J = 2.7 Hz), 7.22-7.16 (2H, m), 7.12 (1H, t, J = 8.0 Hz), 6.91 (1H, td, J = 7.4, 1.2 Hz), 6.85 (1H, d, J = 8.1 Hz), 6.70 (1H, d, J = 7.8 Hz), 6.34 (1H, d, J = 15.5 Hz), 5.90 (1H, t, J = 5.5 Hz), 3.95 (3H, s), 3.56 (2H, d, J = 6.8 Hz), 3.72- 3.65 (2H, m), 2.95 (2H, t, J = 6.6 Hz), 1.38- 1.28 (1H, m), 0. [symbol] 9-0.60 (2H, m), 0.41-0.34 (2H, m). |
| 2 | 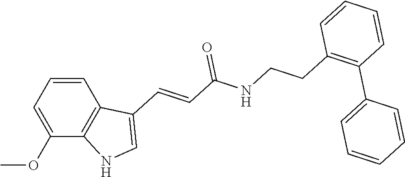 | 19 | NMR2(400 MHz); 8.60 (1H, s), 7.78 (1H, d, J = 15.5 Hz), 7.48-7.21 (11H, m), 7.15 (1H, t, J = 7.9 Hz), 6.75-6. [symbol] (1H, m), 6.24 (1H, d, J = 15.6 Hz), 5.39-5.31 (1H, m), 3.96 (3H, s), 3.52-3.43 (2H, m), 2.91 (2H, t, J = 7.1 Hz). |
| 3 | 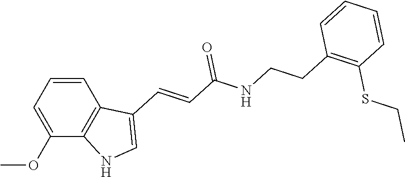 | 17 | NMR2(500 MHz); 8.84 (1H, s), 7.84 (1H, d, J = 15.6 Hz), 7.45 (1H, d, J = 8.1 Hz), 7.40 (1H, d, J = 2.7 Hz), 7.34-7.31 (1H, m), 7.25-7.18 (2H, m), 7.38-7.10 (2H, m), 6.71 (1H, d, J = 7.8 Hz), 6.35 (1H, d, J = 15.6 Hz), 5. [symbol] (1H, t, J = 6.8 Hz), 3.96 (3H, s), 3.72-3.65 (2H, m), 3.06 (2H, t, J = 6.9 Hz), 2.95 (2H, q, J = 7.3 Hz), 1.34 (3H, t, J = 7.4 Hz). |
| 4 | 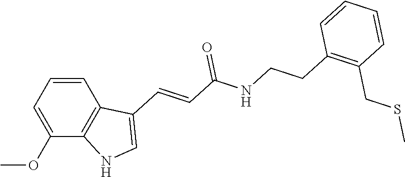 | 17 | NMR2(500 MHz); 8.67 (1H, s), 7.84 (1H, d, J = 15.5 Hz), 7.43 (1H, d, J = 8.0 Hz), 7.39 (1H, d, J = 2.7 Hz), 7.29-7.09 (5H, m), 6.70 (1H, d, J = 7.7 Hz), 6.36 (1H, d, J = [symbol] Hz), 5.99- 5.93 (1H, m), 3.95 (3H, s), 3.78 (2H, s), 3.75- 3. [symbol] (2H, m), 3.01 (2H, t, J = 7.0 Hz), 2.09 (3H, s). |

[symbol]

indicates data missing or illegible when filed

TABLE 2-2

| EX | STR | Prop | Data |
|---|---|---|---|
| 5 | 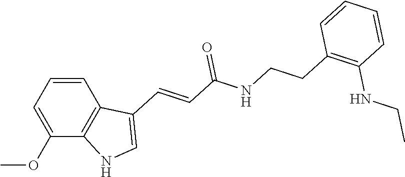 | 17 | NMR2(600 MHz); 8.68 (1H, s), 7.86 (1H, d, J = 15.6 Hz), 7.45 (1H, d, J = 8.1 Hz), 7.40 (1H, d, J = 2.7 Hz), 7.20-7.11 (2H, m), 7.03 (1H, dd, J = 7.4, 1.6 Hz), 6.71 (1H, d, J = 7.8 Hz), 6.88-6.82 (2H, m), 6.38 (1H, d, J = 15.8 Hz), 5.93 (1H, J = 6.1 Hz), 4.56 (1H, brs), 3.96 (3H, s), 3.58-3.50 (2H, m), 3.28-3.20 (2H, m), 2.84-2.77 (2H, m), 1.35 (3H, t, J = 7.1 Hz). |
| 6 | 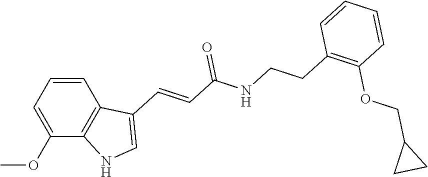 | 17 | NMR2(500 MHz); 8.68-8.61 (1H, m), 8.02- 7.79 (1H, m), 7.50-7.3 [symbol] (2H, m), 7.24-7.10 (3H, m), 6.96-6.68 (4H, m), 3.97 (3H, s) 3.88- 3.80 (2H, m), 3.77-3.71 (2H, m), 3.21- 3.09 (3H, m), 3.07-2.93 (2H, m), 1.34-1.20 (1H, m), 0.68-0.53 (2H, m), 0.44-0.26 (2H, m). |
| 7 | 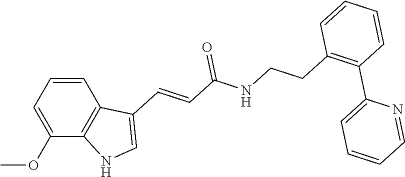 | 17 | NMR2(500 MHz); 8.94-8.89 (1H, m), 8.76- 8.71 (1H, m), 8.61 (1H, s), 7.85 (1H, td, J = 7.7, 1.8 Hz), 7.77 (1H, d, J = 15.8 Hz) 7.52 (1H, d, J = 7.8 Hz), 7.46-7.29 (7H, m), 7.07 (1H, t, J = 7.9 Hz), 6.88 (1H, d, J = 7.8 Hz), 6.47 (1H, d, J = 15.7 Hz), 3.94 (3H, s), 3.77- 3.88 (2H, m), 2.92-2.86 (2H, m). |
| 8 | 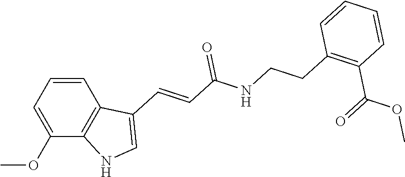 | 17 | NMR2(500 MHz); 8.65 (1H, s), 7.89 (1H, dd, J = 7.9, 1.5 Hz), 7.81 (1H, d, J = 15.6 Hz), 7.50- 7.42 (2H, m), 7.40 (1H, d, J = 2.7 Hz), 7.34 (1H, d, J = 7.8 Hz), 7.29 (1H, td, J = 7.6, 1.3 Hz), 7.14 (1H, t, J = 7.9 Hz), 6.71 (1H, d, J = 7.7 Hz), 6.34 (1H, d, J = 15.6 Hz), 6.29-6.23 (1H, m), 3.96 (3H, s), 3.93 (3H, s), 3.74-3.67 (2H, m), 3. 22 (2H, t, J = [symbol] Hz). |

[symbol]

indicates data missing or illegible when filed

TABLE 2-3

| EX | STR | Prop | Data |
|---|---|---|---|
| 9 | 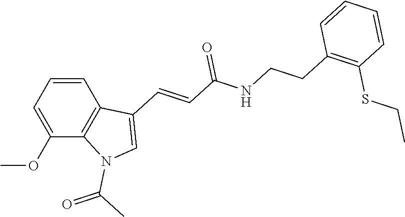 | 2 [symbol] | NMR2(500 MHz); 7.82 (1H, s), 7.74 (1H, d, J = 15.5 Hz), 7.42 (1H, d, J = 7.9 Hz), 7.36-7.12 (5H, m), 6.90 (1H, d, J = 7.9 Hz), 6.39 (1H, d, J = 15.7 Hz), 5.73 (1H, (, t, J = 5.9 Hz), 3.9 [symbol] (3H, s), 3.72-3.65 (2H, m), 3.06 (2H, t, J = 6.9 Hz), 2.96 (2H, q, J = 7.4 Hz), 2.68 (3H, s) 1.34 (3H, t, J = 7.3 Hz). |
| 10 | 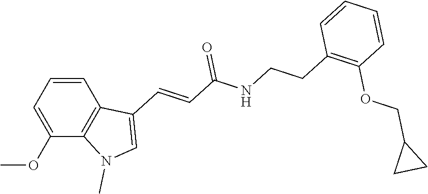 | 10 | NMR2(500 MHz); 7.96-7.75 (1H, m), 7.46- 7.36 (1H, m), 7.24-7.06 (4H, m), 6.92-6.85 (1H, m), 6.83-6.71 (2H, m), 6.68 (1H, d, J = 7.7 Hz), 4.05 (3H, s), 3.93(3H, s), 3.88-3.81 (2H, m), 3.72 (2H, t, J = 7.4 Hz), 3.18-3.08 (3H, m), 3,04-2.94 (2H, m), 1.38-1.23 (1H, m), 0.68-0.54 (2H, m), 0.42-0.28 (2H, m). |
| 11 | 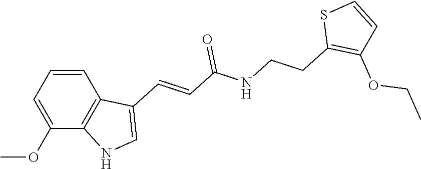 | 17 | NMR2(500 MHz); 8.61 (1H, s), 7.83 (1H, d, J = 15.6 Hz), 7.46 (1H, d, J = 8.1 Hz), 7.40 (1H, d, J = 2.7 Hz), 7.14 (1H, t, J = 7.9 Hz), 7.04 (1H, d, J = 5.5 Hz), 6.83 (1H, d, J = 5.5 Hz), 6.71 (1H, d, J = 7.7 Hz), 6.36 (1H, d, J = 15.8 Hz), 5.98 (1H, t, J = 5.8 Hz), 4.08 (2H, q, J = 7.0 Hz), 3.86 (3H, s), 3.66-3.58 (2H, m), 2.99 (2H, t, J = 6.4 Hz), 1.40 (3H, t, J = 7.0 Hz). |
| 12 | 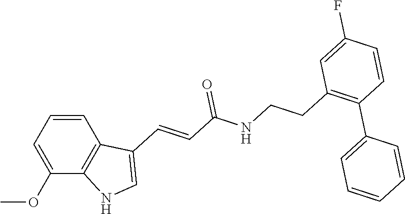 | 19 | NMR2(500 MHz); 8.80 (1H, s), 7.78 (1H, d, J = 15.5 Hz), 7.46-7.34 (5H, m), 7.32-7.28 (2H, (m), 7.20 (1H, dd, J = 8.5, 5.9 Hz), 7.15 (1H, t, J = 7.9 Hz), 7.07 (1H, dd, J = 9.8, 2.7 Hz), 6.98 (1H, td, J = 8.3, 2.7 Hz), 6.72 (1H, d, J = 7.7 Hz), 6.25 (1H, d, J = 15.6 Hz), 5.36 (1H, t, J = 5.3 Hz), 3.96 (3H, s), 3.51-3.44 (2H, m), 2.88 (2H, t, J = 7.1 Hz). |

[symbol]

indicates data missing or illegible when filed

TABLE 2-4

| EX | STR | Prop | Data |
|---|---|---|---|
| 13 | 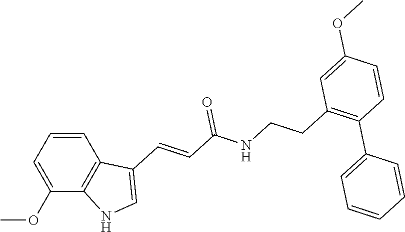 | 19 | NMR2(500 MHz); 8.58 (1H, s), 7.78 (1H, d, J = 15.6 Hz), 7.48-7.37 (4H, m), 7.37-7.28 (3H, m), 7.20-7.11 (2H, m), 6.90 (1H, d, J = 2.6 Hz), 6.84 (1H, dd, J = 8.4, 2.7 Hz), 6.72 (1H, d, J = 7.7 Hz), 6.25 (1H, d, J = 15.6 Hz), 5.36 (1H, t, J = 5.3 Hz), 3.97 (1H, s), 3.84 (3H, s), 3.52-3.44 (2H, m), 2.89 (2H, t, J = 7.1 Hz). |
| 14 | 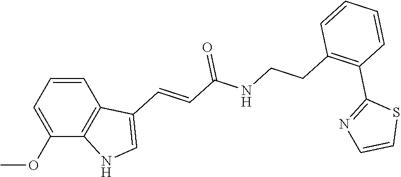 | 17 | NMR2(800 MHz); 8.58 (1H, s), 8.19 (1H, s), 7.98 (1H, d, J = 3.4 Hz), 7.75 (1H, d, J = 1 [symbol] .7 Hz), 7.60-7.54 (1H, m), 7.50-7.37 (4H, m), 7.37-7.28 (2H, m), 7.09 (1H, t, J = 7.9 Hz), 6.6 [symbol] (1H, d, J = 7.7 Hz), 6.40 (1H, d, J = 15.8 Hz), 3.96 (3H, s), 3.79-3.72 (2H, m), 3.14- 3.08 (2H, m). |
| 15 | 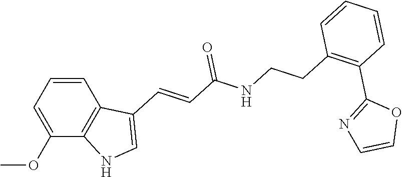 | 17 | NMR2(500 MHz); 8.62 (1H, s), 7.90 (1H, dd, J = 7.7, 1.4 Hz), 7.80 (1H, d, J = 0.9 Hz), 7.75 (1H, d, J = 15.8 Hz), 7.46-7.30 (7H, m), 7.11 (1H, t, J = 7.9 Hz), 6.89 (1H, d, J = 7.7 Hz), 6.33 (1H, d, J = 15.7 Hz), 3.95 (3H, s), 3.80- 3.73 (2H, m), 3.35-3.29 (2H, m) |
| 18 | 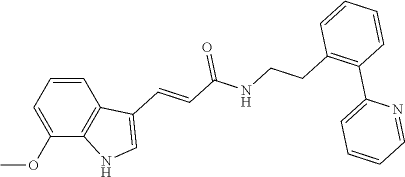 | 17 | NMR2(500 MHz); 8.82 (1H, d, J = 1.6 Hz), 8.70- 8.66 (1H, m), 8.66-8.61 (2H, m), 7.77 (1H, d, J = 15.7 Hz), 7.50-7.45 (3H, m), 7.44- 7.34 (4H, m), 7.10 (1H, t, J = 7.9 Hz), 6.89 (1H, d, J = 7.7 Hz), 6.38 (1H, d, J = 15.7 Hz), 3.95 (3H, s), 3.75-3.68 (2H, m), 2.97-2.90 (2H, m) |
| 17 | 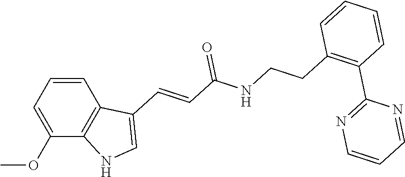 | 17 | NMR2(500 MHz); 8.00 (2H, d, J = 4.9 Hz), 8.75 (1H, s), 7.88-7.77 (2H, m), 7.76 (1H, d, J = 15.7 Hz), 7.48-7.33 (5H, m), 7.29 (1H, t, J = 4.9 Hz), 7.08 (1H, t, J = 7.9 Hz), 6.68 (1H, d, J = 7.8 Hz), 6.35 (1H, d, J = 15.7 Hz), 3.94 (3H, s), 3.79-3.72 (2H, m), 3.11-3.06 (2H, m). |

[symbol]

indicates data missing or illegible when filed

TABLE 2-5

| EX | STR | Prop | Data |
|---|---|---|---|
| 18 | 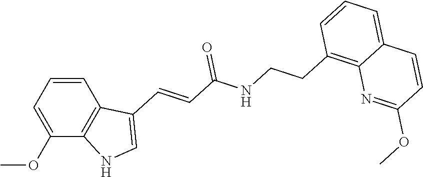 | 45 | NMR2(500 MHz); 8.55 (1H, s), 8.02 (1H, d, J = 8.8 Hz), 7.72 (1H, d, J = 15.7 Hz), 7.84 (1H, dd, J = 8.0, 1.5 Hz), 7.57-7.54 (1H, m), 7.41- 7.32 (3H, m), 7.14 (1H, t, J = 7.9 Hz), 6.96 (1H, d, J = 5.8 Hz), 6.71 (1H, d, J = 7.8 Hz), 6.32-6.29 (1H, m), 6.25 (1H, d, J = 15.7 Hz), 4.15 (3H, s), 3.96 (3H, s), 3.89-3.81 (2H, m), 3.46 (2H, t, J = 6.4 Hz). |
| 19 | 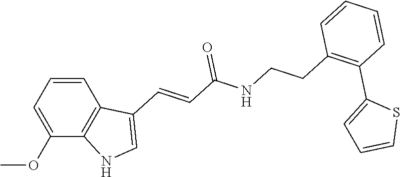 | 19 | NMR2(500 MHz); 8.59 (1H, s), 7.81 (1H, d, J = 15.6 Hz), 7.48-7.26 (7H, m), 7.18-7.07 (3H, m), 6.72 (1H, d, J = 7.7 Hz), 6.29 (1H, d, J = 15.6 Hz), 5.47 (1H, t, J = 5.9 Hz), 3.97 (3H, s), 3.61-3.54 (2H, m), 3.05 (2H, t, J = 7.1 Hz). |
| 20 | 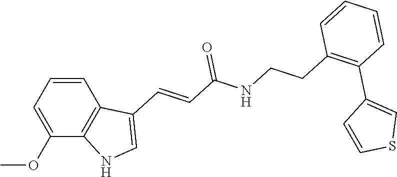 | 19 | NMR2(500 MHz); 8.61 (1H, s), 7.80 (1H, d, J = 15.6 Hz), 7.44 (1H, d, J = 8.1 Hz), 7.43- 7.38 (2H, m), 7.37-7.23 (5H, m), 7.18-7.11 (2H, m), 6.72 (1H, d, J = 7.7 Hz), 6.27 (1H, d, J = 15.6 Hz), 5.43 (1H, t, J = 5.6 Hz), 3.97 (3H, s), 3.58-3.48 (2H, m), 2.97 (2H, t, J = 7 [symbol] Hz). |
| 21 | 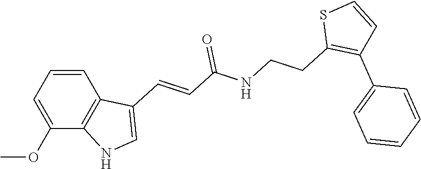 | 19 | NMR2(500 MMx); 8.60 (1H, s), 7.78 (1H, d, J = 15.5 Hz), 7.47-1.30 (6H, m), 7.35-7.28 (1H, m), 7.22 (1H, d, J = 5.2 Hz), 7.18 (1H, t, J = 7.9 Hz), 7.08 (1H, d, J = 5.1 Hz), 6.72 (1H, d, J = 7.7 Hz), 6.25 (1H, d, J = 15.6 Hz), 5.59- 5.56 (1H, m), 3.97 (3H, s), 3.67-3.60 (2H, m), 3.19 (2H, t, J = 6.7 Hz). |
| 22 | 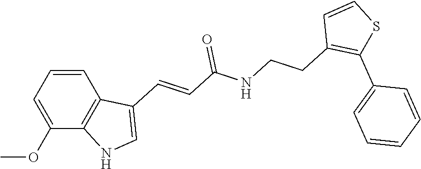 | 45 | NMR2(400 MHz); 8.58 (1H, s), 7.78 (1H, d, J = 15.8 Hz), 7.50-7.39 (8H, m), 7.38-7.28 (2H, m), 7.15 (1H, t, J = 7.9 Hz), 7.05 (1H, d, J = 5.2 Hz), 6.72 (1H, d, J = 7.7 Hz), 6.24 (1H, d, J = 15.6 Hz), 5.45 (1H, m), 3.97 (3H, s), 3.67- 3.57 (2H, m), 2.99 (2H, t, J = 7.0 Hz). |

[symbol]

indicates data missing or illegible when filed

TABLE 2-6

| EX | STR | Prop | Data |
|---|---|---|---|
| 23 | 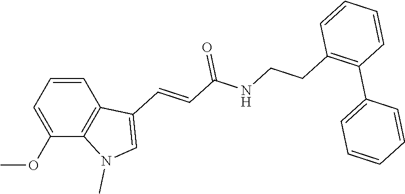 | 10 | NMR2(500 MHz); 7.73 (1H, d, J = 15.5 Hz), 7.43-7.24 (10H, m), 7.14 (1H, s), 7.09 (1H, t, J = 7.9 Hz), 6.67 (1H, d, J = 7.8 Hz), 6.16 (1H, d, J = 15.5 Hz), 5.34-5.28 (1H, m), 4.04 (3H, s), 3.92 (3H, s), 3.51-3.43 (2H, m), 2.90 (2H, t, J = 7.1 Hz). |
| 24 | 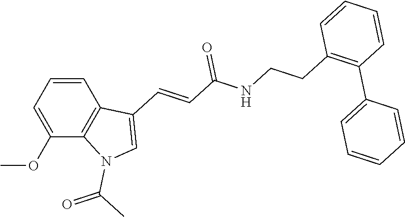 | 24 | NMR2(500 MHz); 7.81 (1H, s), 7.68 (1H, d, J = 15.7 Hz), 7.47-7.22 (11H, m), 6.9 [symbol] (1H, d, J = 8.0 Hz), 6.29 (1H, d, J = 15.7 Hz), 5.41- 5.37 (1H, m), 3.96 (3H, s), 3.51-3.43 (2H, m), 2.92 (2H, t, J = 7.1 Hz), 2.67 (3H, s). |
| 25 | 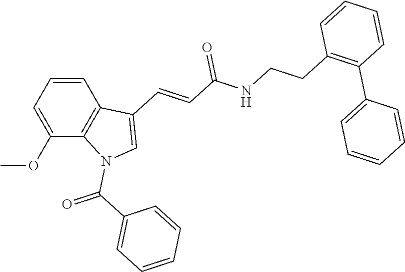 | 2 [symbol] | NMR2(500 MHz); 7.79-7.73 (2H, m), 7.69 (1H, d, J = 15.6 Hz), 7.65-7.57 (2H, m), 7.51- 7.40 (5H, m), 7.39-7.22 (8H, m), 6.79 (1H, d, J = 7.9 Hz) 6.30 (1H, d, J = 15.6 Hz), 5.38 (1H, t, J = 6.6 Hz), 3.67 (3H, s), 3.51-3.48 (2H, m), 2.91 (2H, t, J = 7.1 Hz). |
| 26 | 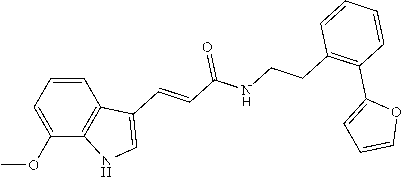 | 19 | NMR2(500 MHz); 8.57 (1H, s), 7.83 (1H, d, J = 15.6 Hz), 7.63-7.66 (1H, m), 7.56-7.51 (1H, m), 7.44 (1H, d, J = 8.1 Hz), 7.40 (1H, d, J = 2.7 Hz), 7.34-7.23 (3H, m), 7.14 (1H, t, J = 7.9 Hz), 6.71 (1H, d, J = 7.8 Hz), 6.64- 6.59 (1H, m), 6.52 (1H, dd, J = 3.4, 1.8 Hz), 6.02 (1H, d, J = 15.6 Hz), 5.62-5.59 (1H, m), 3.97 (3H, s), 3.71-3.63 (2H, m), 3.13 (2H, t, J = 7.0 Hz). |
| 27 | 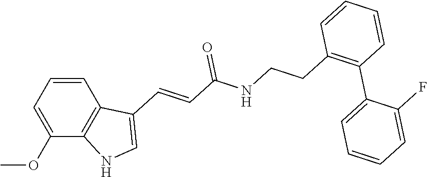 | 19 | NMR2(500 MHz); 8.57 (1H, s), 7.77 (1H, d, J = 15.6 Hz), 7.45 (1H, d, J = 8.0 Hz), 7.41- 7.10 (10H, m), 6.71 (1H, d, J = 7.7 Hz), 6.27 (1H, d, J = 15.6 Hz), 5.41-5.38 (1H, m), 3.96 (3H, s), 3.63-3.48 (2H, m), 2.81 (2H, t, J = 6.9 Hz). |

[symbol]

indicates data missing or illegible when filed

TABLE 2-7

| EX | STR | Prop | Data |
|---|---|---|---|
| 28 | 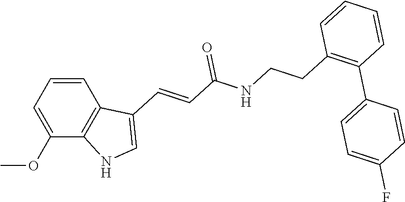 | 19 | NMR2(500 MHz); 8.57 (1H, s), 7.79 (1H, d, J = 15.5 Hz), 7.46-7.07 (11H, m), 6.72 (1H, d, J = 7.7 Hz), 6.26 (1H, d, J = 15.6 Hz), 5.39- 5.35 (1H, m), 3.97 (3H, s), 3.52-3.44 (2H, m), 2.88 (2H, t, J = 7.2 Hz). |
| 29 | 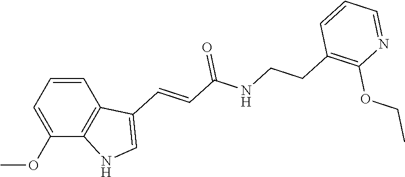 | 29 | NMR2(500 MHz); 8.57 (1H, s), 8.05 (1H, dd, J = 8.0, 1.9 Hz), 7.83 (1H, d, J = 15.6 Hz), 7.47- 7.38 (3H, m), 7.14 (1H, [symbol] , J = 7.9 Hz), 6.83 (1H, dd, J = 7.2, 5.0 Hz), 6.72 (1H, d, J = 7.8 Hz), 6.34 (1H, d, J = 15.6 Hz), 5.73-5.70 (1H, m), 4.43 (2H, q, J = 7.1 Hz), 3.97 (3H, s), 3.70- 3.63 (2H, m), 2.88 (2H, t, J = 6.7 Hz), 1.44 (3H, t, J = 7.0 Hz). |
| 30 | 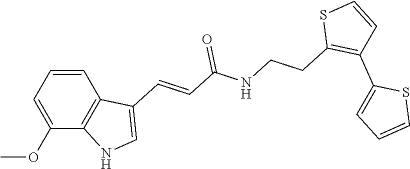 | 17 | NMR2(500 MHz); 8.58 (1H, s), 7.82 (1H, d, J = 15.6 Hz), 7.45 (1H, d, J = 8.1 Hz), 7.41 (1H, d, J = 2.7 Hz), 7.28 (1H, dd, J = 5.2, 1.2 Hz), 7.21-7.12 (4H, m), 7.08 (1H, dd, J = 5.2, 3.5 Hz), 6.72 (1H, d, J = 7.8 Hz), 6.30 (1H, d, J = 15.6 Hz), 5.67-5.63 (1H, m), 3.97 (3H, s), 3.74-3.66 (2H, m), 3.29 (2H, t, J = 6.7 Hz). |
| 31 | 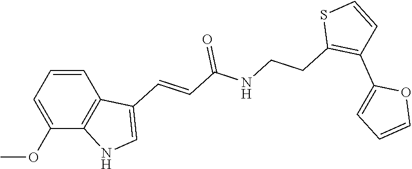 | 45 | NMR2(500 MHz); 8.68 (1H, s), 7.83 (1H, d, J = 15.6 Hz) 7.47-7.39 (3H, m), 7.24 (1H, d, J = 5. [symbol] Hz), 7.19-7.09 (2H, m), 6.71 (1H, d, J = 7.7 Hz), 6.54 (1H, d, J = 3.4 Hz), 6.47 (1H, dd, J = 3.4, 1.8 Hz), 6.31 (1H, d, J = 15.7 Hz), 5.75-5.72 (1H, m), 3.96 (3H, s), 3.77-3.6 [symbol] (2H, m), 3.35 (2H, t, J = 6.7 Hz). |

[symbol]

indicates data missing or illegible when filed

TABLE 2-8

| EX | STR | Prop | Data |
|---|---|---|---|
| 32 | 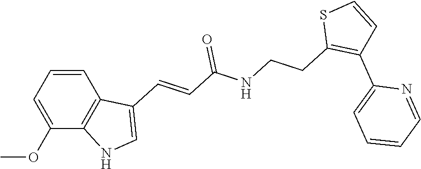 | 45 | NMR2(500 MHz); 9.02 (1H, s), 8.75-8.70 (1H, m), 8.52 (1H, s), 7.82 (1H, t, J = 7.8, 1.8 Hz), 7.76 (1H, d, J = 16.8 Hz), 7.59-7.54 (1H, m), 7.38-7.33 (2H, m), 7.33-7.27 (1H, m), 7.24 (1H, d, J = 5.2 Hz), 7.19 (1H, d, J = 5.2 Hz), 7.08 (1H, t, J = 7.9 Hz), 6.68 (1H, d, J = 7.8 Hz), 6.44 (1H, d, J = 15.8 Hz), 3.95 (3H, s), 3.79-3.72 (2H, m), 3.34-3.28 (2H, m). |
| 33 | 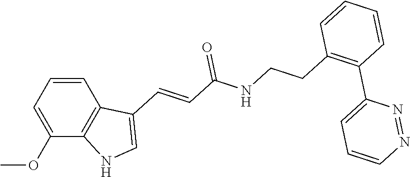 | 17 | NMR2(500 MHz); 9.28-9.23 (1H, m), 8.56 (1H, s), 8.18-8.11 (1H, m), 7.80 (1H, d, J = 15.8 Hz), 7.71 (1H, dd, J = 8.5, 1.7 Hz), 7.68- 7.62 (1H, m), 7.53 (1H, d, J = 8.1 Hz), 7.51- 7.43 (2H, m), 7.43-7.32 (3H, m), 7.10 (1H, t, J = 7.9 Hz), 6.68 (1H, d, J = 7.7 Hz), 6.50 (1H, d, J = 15.7 Hz), 3.94 (3H, s), 3.78-3.71 (2H, m), 2.98-2.92 (2H, m) |
| 34 | 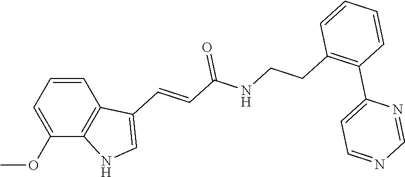 | 17 | NMR2(500 MHz); 9.3 [symbol] (1H, d, J = 1.4 Hz), 8.87 (1H, d, J = 5.2 Hz), 8.85 (1H, s), 7.81-7.78 (2H, m), 7.55 (1H, dd, J = 5.2, 1.4 Hz), 7.51- 7.44 (2H, m), 7.44-7.33 (4H, m), 7.10 (1H, t, J = 7.8 Hz), 6.69 (1H, d, J = 7.8 Hz), 6.38 (1H, d, J = 15.7 Hz), 3.95 (3H, s), 3.77-3.70 (2H, m), 3.01-2.95 (2H, m). |
| 35 | 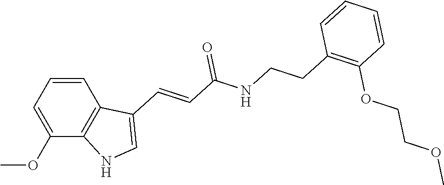 | 17 | NMR2(400 MHz); 8.56 (1H, s), 7.77 (1H, d, J = 15.7 Hz), 7.51 (1H, d, J = 8.2 Hz), 7.3 [symbol] (1H, d, J = 2.7 Hz), 7.24-7.16 (2H, m), 7.13 (1H, t, J = 7.9 Hz), 6.93 (1H, td, J = 7.5, 1.1 Hz), 6.8 [symbol] (1H, dd, J = 8.6, 1.1 Hz), 6.71 (1H, d, J = 7.6 Hz), 6.48 (1H, d, J = 15.6 Hz), 6.35-6.28 (1H, m), 4.22-4.16 (2H, m), 3.96 (3H, s), 3.89- 3.83 (2H, m), 3.70-3.61 (2H, m), 3.49 (3H, s), 2.99-2.91 (2H, m). |

[symbol]

indicates data missing or illegible when filed

TABLE 2-9

| EX | STR | Prop | Data |
|---|---|---|---|
| 36 | 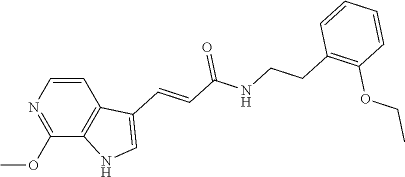 | 17 | NMR2(400 MHz); 8.79 (1H, s), 7.85 (1H, d, J = 5.8 Hz), 7.77 (1H, d, J = 18.7 Hz), 7.45 (1H, d, J = 2.8 Hz), 7.33-7.30 (1H, m), 7.24-7.15 (2H, m), 6. [symbol] -6.85 (2H, m), 6.30 (1H, d, J = 15. [symbol] Hz), 5.87-5.81 (1H, m), 4.14-4. [symbol] 4 (5H, m), 3.73-3.62 (2H, m), 2.9 [symbol] (2H, t, J = [symbol] Hz), 1.47 (3H, t, J = 7.0 Hz). |
| 37 | 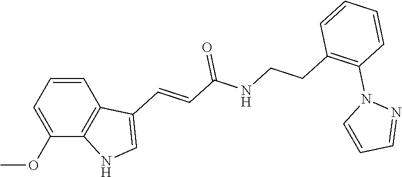 | 17 | NMR2(500 MHz); 8.56 (1H, s), 7.81 (1H, d, J = 1.9 Hz), 7.77 (1H, d, J = 15.7 Hz), 7.74 (1H, s), 7.69-7.65 (1H, m), 7.49-7.39 (3H, m), 7.37 (1H, d, J = 2.7 Hz), 7.36-7.29 (1H, m), 7.28 (1H, dd, J = 7.9, 1.5 Hz), 7.12 (1H, t, J = 7.5 Hz), 6.69 (1H, d, J = 7.8 Hz), 6.51 (1H, t. J = 2.2 Hz), 6.44 (1H, d, J = 15.8 Hz), 3.95 (3H, s), 3.68-3.62 (2H, m), 2.81-2.74 (2H, m). |
| 38 | 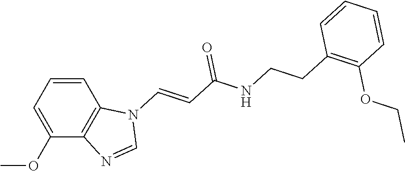 | 17 | NMR2(400 MHz); 8.10 (1H, d, J = 13.9 Hz), 8.05 (1H, s), 7.31 (1H, t, J = 8.1 Hz), 7.27- 7.13 (3H, m), 6.9 [symbol] -6.88 (2H, m), 6.79 (1H, d, J = 8.0 Hz), 6.1 [symbol] (1H, d, J = 13.9 Hz), 5.98- 5.88 (1H, m), 4.09 (2H, q, J = 7.0 Hz), 4.04 (3H, s), 3.72-3.6 [symbol] (2H, m), 2.93 (2H, t, J = 6.6 Hz), 1.46 (3H, t, J = 7.0 Hz). |
| 39 | 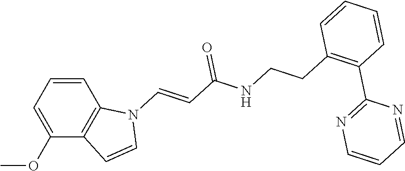 | 17 | NMR2(500 MHz); 8.91 (2H, d, J = 4.9 Hz), 8.16 (1H, d, J = 13.7 Hz), 8.04 (1H, s), 7.80 (1H, dd, J = 7.8, 1.4 Hz), 7.60-7.31 (4H, m), 7.24- 7.18 (2H, m), 7.18 (1H, d, J = 8.2 Hz), 6.78 (1H, d, J = 3.5 Hz), 6.64-6.59 (1H, m), 5.83 (1H, d, J = 13.7 Hz), 3.94 (3H, s), 3.80-3.73 (2H, m), 3.10-3.04 (2H, m) |

[symbol]

indicates data missing or illegible when filed

TABLE 2-10

| EX | STR | Prop | Data |
|---|---|---|---|
| 40 | 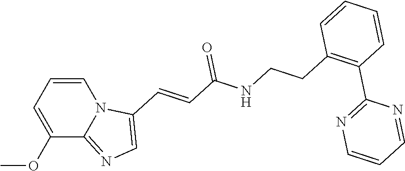 | 45 | NMR1(400 MHz); 8.94 (2H, d, J = 4.9 Hz), 8.27 (1H, dd, J = 7.0. 0.9 Hz), 8.17 (1H, t, J = 5.5 Hz), 7.92 (1H, s), 7.76 (1H, dd, J = 7.7, 1.4 Hz), 7.65 (1H, d, J = 15.8 Hz), 7.52-7.32 (4H, m), 7.02-6.94 (1H, m), 6.82-6.77 (1H, m), 6.5 [symbol] (1H, d, J = 15.9 Hz), 3.95 (3H, s), 3.45- 3.31 (2H, m), 3.13-3. [symbol] 5 (2H, m). |
| 41 | 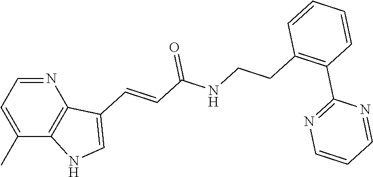 | 45 | NMR2(400 MHz); 9.33 (1H, s), 8.99 (2H, d, J = 4.9 Hz), 8.37 (1H, d, J = 4.8 Hz), 8.04 (1H, t, J = 4.4 Hz), 7.80-7.75 (1H, m), 7.72 ( [symbol] H, d, J = 15.3 Hz), 7.50 (1H, d, J = 2.8 Hz), 7.4 [symbol] - 7.28 (5H, m), 6.9 [symbol] (1H, dd, J = 4.8. 0.9 Hz), 3.80-3.71 (2H, m), 3.12-3.04 (2H, m), 2.51 (3H, s) |
| 42 | 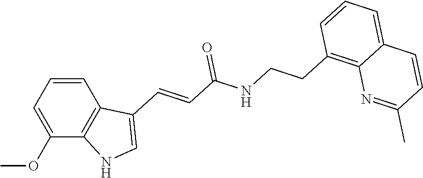 | 45 | NMR2(500 MHz); 8.57 (1H, s), 8.07 (1H, d, J = 5.3 Hz), 7.77-7.66 (2H, m), 7.62-7.57 (1H, m), 7.53-7.50 (1H, m), 7.46-7.38 (2H, m), 7.36-7.29 (2H, m), 7.13 (1H, t, J = 7.9 Hz), 6.71 (1H, d, J = 7.8 Hz), 6.29 (1H, d, J = 15.6 Hz), 3.96 (3H, s), 3.83-3.78 (2H, m), 3.54-3.48 (2H, m), 2.88 (3H, s). |
| 43 | 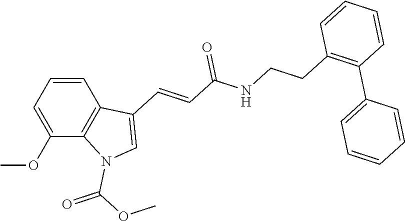 | 25 | NMR2(400 MHz); 7.74 (1H, s), 7.67 (1H, d, J = 15.7 Hz), 7.47-7.22 (11H, m), 6.91 (1H, d, J = 7.3 Hz), 6.29 (1H, d, J = 15.7 Hz), 5.38 (1H, t, J = 5.6 Hz), 4.02 (3H, s), 3.97 (3H, s), 3.52-3.42 (2H, m), 2.91 (2H, t, J = 7.1 Hz). |
| 44 | 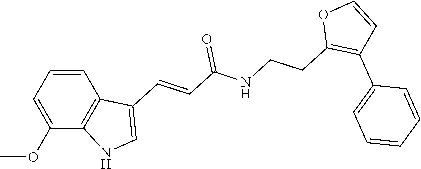 | 45 | NMR2(400 MHz); 8.58 (1H, s), 7.80 (1H, d, J = 15.6 Hz), 7.47-7.34 (7H, m), 7.28-7.23 (1H, m), 7.15 (1H, t, J = 7.9 Hz), 6.72 (1H, d, J = 7.7 Hz), 6.54 (1H, d, J = 1.9 Hz), 6.26 (1H, d, J = 15.6 Hz), 5.70-5.66 (1H, m), 3.97 (3H, s), 3.79-3.69 (2H, m), 3.10 (2H, t, J = 6.5 Hz). |

[symbol]

indicates data missing or illegible when filed

TABLE 2-11

| EX | STR | Prop | Data |
|---|---|---|---|
| 45 | 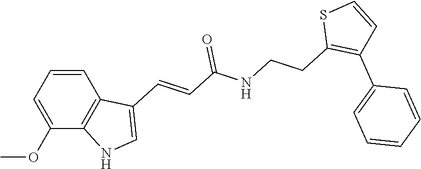 | 45 | NMR2(500 MHz); 8.83 (2H, d, J = 4.9 Hz), 8.58 (1H, s), 7.76 (1H, d, J = 15.6 Hz), 7.70 (1H, d, J = [symbol] .3 Hz), 7.42-7.31 (3H, m), 7.24-7.1 [symbol] (2H, m), 7.09 (1H, t, J = 7.9 Hz), 6.7 [symbol] (1H, d, J = 7.8 Hz), 6.30 (1H, d, J = 15.7 Hz), 3.98 (3H, s), 3.84-3.77 (2H, m), 3.5 [symbol] -3.52 (2H, m). |
| 46 | 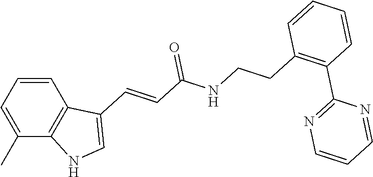 | 45 | NMR2(500 MHz); 8.91 (2H, d, J = 5.0 Hz), 8.51 (1H, s), 7.96-7.91 (1H, m), 7.82-7.74 (2H, m), 7.64 (1H, d, J = 7.9 Hz), 7.50-7.34 (4H, m), 7.31 (1H, t, J = 4.9 Hz), 7.09 (1H, t, J = 7.5 Hz), 7.04 (1H, d, J = 7.2 Hz), 6.38 (1H, d, J = 15.7 Hz), 3.80-3.73 (2H, m), 3.11-3.08 (2H, m), 2.49 (3H, s). |
| 47 | 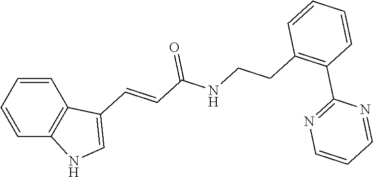 | 47 | NMR2(500 MHz); 8. [symbol] 1 (2H, d, J = 4.9 Hz), 8.52 (1H, s), 7.95-7.91 (1H, m), 7.83-7.74 (3H, m), 7.48-7.3 [symbol] (5H, m), 7.32 (1H, t, J = 4.9 Hz), 7.26-7.14 (2H, m), 6.38 (1H, d, J = 15.6 Hz, 3.81-3.74 (2H, m), 3. [symbol] 1-3.05 (2H, m). |
| 48 | 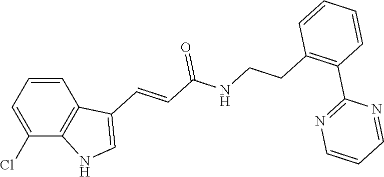 | 45 | NMR2(500 MHz); 8.91 (2H, d, J = 4 .9 Hz), 8.64 (1H, s), 7.98-7.94 (1H, m), 7.83-7.77 (1H, m), 7.74 (1H, d, J = 15.8 Hz), 7.68 (1H, d, J = 8.0 Hz), 7.48-7.38 (4H, m), 7.33 (1H, t, J = 4.9 Hz), 7.24 ( (1H, d, J = 7.6 Hz), 7.11 (1H, t, J = 7.9 Hz), 6.38 (1H, d, J = 15.7 Hz), 3.81- 3.74 (2H, m), 3.11-3.05 (2H, m). |
| 49 | 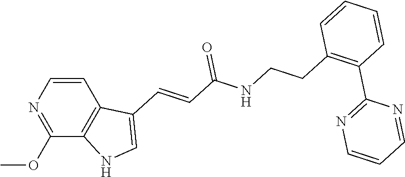 | 45 | NMR2(500 MHz); 8.92 (2H, d, J = 4.9 Hz), 8.76 (1H, s) 8.07-8.03 (1H, m), 7.83-7.77 (2H, m), 7.72 (1H, d, J = 15.7 Hz), 7.50-7.32 (5H, m), 7.25 (1H, d, J = 5.7 Hz), 6.33 (1H, d, J = 16.7 Hz), 4.09 (3H, s), 3.81-3.74 (2H, m), 3.10-3.04 (2H, m). |

[symbol]

indicates data missing or illegible when filed

TABLE 2-12

| EX | STR | Prop | Data |
|---|---|---|---|
| 50 | 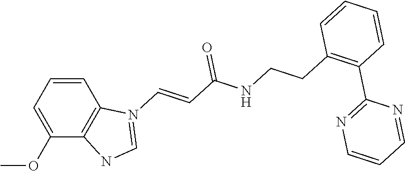 | 17 | NMR1(500 MHz); 8.94 (2H, d, J = 4.9 Hz), 8.53 (1H, s), 8.20 (1H, t, J = 5.6 Hz), 8.06 (1H, d, J = 14.2 Hz), 7. [symbol] (1H, d, J = 7.7 Hz), 7.62- 7.30 (6H, m), 6.90 (1H, d, J = 8.0 Hz), 6. [symbol] 0 (1H, d, J = 14.3 Hz), 3.96 (3H, s), 3.46-3.38 (2H, m), 3. [symbol] (2H, t, J = 7.3 Hz). |
| 51 | 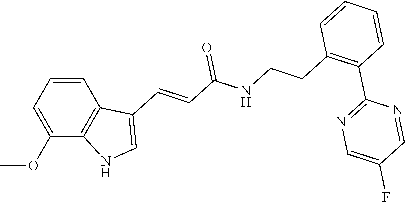 | 17 | NMR2(500 MHz); 8.73 (2H, s), 8.63 (1H, s), 7.81-7.73 (2H, m), 7.50-7.33 (5H, m), 7.11 (1H, t, J = 7.9 Hz), 6.94-6.89 (1H, m), 6.70 (1H, d, J = 7.8 Hz), 6.30 (1H, d, J = 15.7 Hz), 3.95 (3H, s), 3.76-3.69 (2H, m), 3.11 (2H, t, J = 8.4 Hz). |
| 52 | 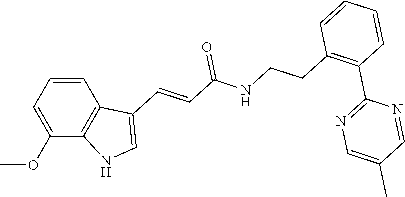 | 17 | NMR2(500 MHz); 8.73 (2H, s), 8.65 (1H, s), 7.97-7.91 (1H, m), 7.79-7.71 (2H, m), 7.47- 7.32 (5H, m), 7.08 (1H, t, J = 7.9 Hz), 6.69 (1H, d, J = 7.8 Hz), 6.36 (1H, d, J = 15.7 Hz), 3.95 (3H, s), 3.78-3.71 (2H, m), 3. [symbol] -3. [symbol] (2H, m), 2.36 (3H, s). |
| 53 | 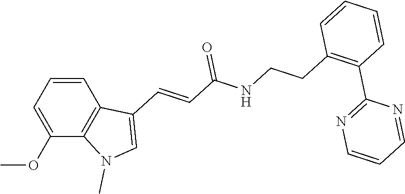 | 10 | NMR2(500 MHz); 8.90 (2H, d, J = 4.9 Hz), 7.79 (1H, d, J = 7.7 Hz), 7.74-7.67 (2H, m), 7.49- 7.27 (5H, m), 7.11 (1H, s), 7.94 (1H, t, J = 7.9 Hz), 6.65 (1H, d, J = 7.8 Hz), 6.26 (1H, d, J = 15.7 Hz), 4.02 (3H, s), 3.91 (3H, s), 3.78- 3.71 (2H, m), 3.11-3.0 [symbol] (2H, m). |
| 54 | 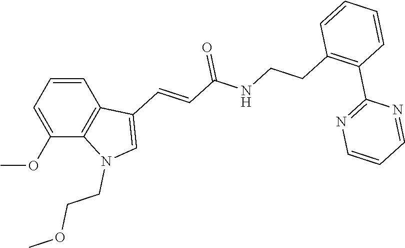 | 10 | NMR2(500 MHz); 8. [symbol] 0 (2H, d, J = 4.9 Hz), 7.80 (1H, d, J = 7.7 Hz), 7.75-7.68 (2H, m), 7.49- 7.34 (4H, m), 7.30 (1H, t, J = 4.9 Hz), 7.25 (1H, s), 7.05 (1H, t, J = 7.9 Hz), 6.67 (1H, d, J = 7.8 Hz), 6.26 (1H, d, J = 15.7 Hz), 4.52 (2H, t, J = 5.4 Hz), 3.92 (3H, s), 3.78-3.71 (2H, m), 3.68 (2H, t, J = 5.4 Hz), 3.27 (3H, s), 3.11- 3.05 (2H, m). |

[symbol]

indicates data missing or illegible when filed

TABLE 2-13

| EX | STR | | Data |
|---|---|---|---|
| 55 | 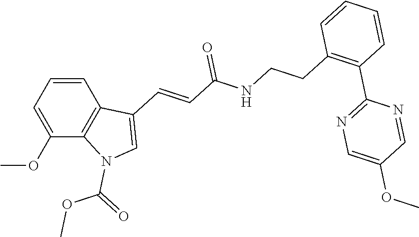 | 25 | NMR2(500 MHz); 8.90 (2H, d, J = 5.0 Hz), 8.10- 8.08 (1H, m), 7.80 (1H, d, J = 7.8 Hz), 7.70 (1H, s), 7.65 (1H, d, J = 15.7 Hz), 7.49-7.30 (5H, m), 7.23 (1H, t, J = 7.9 Hz), 6.89 (1H, d, J = [symbol] .0 Hz), 6.38 (1H, d, J = 15.7 Hz), 4.01 (3H, s), 3.95 (3H, s), 3.80-3.73 (2H, m), 3.10- 3.04 (2H, m). |
| 56 | 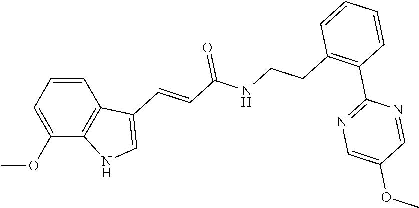 | 45 | NMR2(500 MHz); 8.59-8.54 (3H, m), 7.79- 7.61 (3H, m), 7.48-7.32 (5H, m), 7.09 (1H, t, J = 7.9 Hz). 6.69 (1H, d, J = 7.5 Hz), 6.35 (1H, d, J = 15.7 Hz), 3.95 (3H, s), 3.91 (3H, s), 3.77- 3.71 (2H, m), 3.07 (2H, t, J = 6.2 Hz). |
| 57 | 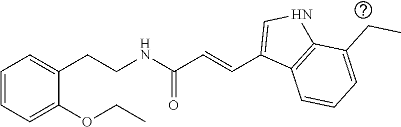 | 17 | NMR2(500 MHz); 8.61 (1H, s), 7.82 (1H, d, J = 15.6 Hz), 7.44 (1H, d, J = 8.1 Hz), 7.40 (1H, d, J = 2.7 Hz), 7.23 (1H, td, J = 7.8, 1.7 Hz), 7.18 (1H, dd, J = 7.4, 1.7 Hz), 7.13 (1H, t, J = 7.9 Hz), 6.95-6.86 (2H, m), 6.71 (1H, d, J = 7.7 Hz), 6.33 (1H, d, J = 15.6 Hz), 5.73 (1H, t, J = 5.6 Hz), 3.96 (3H, s), 3.88 (3H, s), 3.65- 3.61 (2H, m), 2 92 (2H, t, J = 6.7 Hz). |
| 58 | 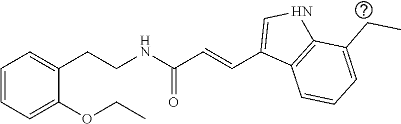 | 10 | NMR2(500 MHz); 7.77 (1H, d, J = 15.6 Hz), 7.40 ( 1H, dd, J = 8.1, 0.8 Hz), 7.26-7.13 (3H, m), 7.08 (1H, t, J = 7.9 Hz), 6.91 (1H, td, J = 7.4, 1.1 Hz), 6.87 (1H, dd, J = 8.2, 1.1 Hz), 6.67 (1H, d, J = 7.7 Hz), 6.25 (1H, d, J = 15.6 Hz), 5.78 (1H, t, J = 5.5 Hz), 4.08 (2H, q, J = 6.9 Hz), 4.04 (3H, s), 3.92 (3H, s), 3.69-3.61 (2H, m), 2.92 (2H, t, J = 6.6 Hz), 1.47 (3H, t, J = 7.0 Hz). |
| 59 | 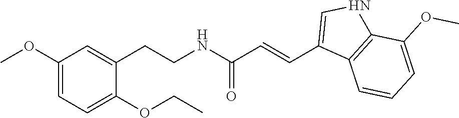 | 17 | NMR2(500 MHz); 8. [symbol] 5 (1H, s), 7.81 (1H, d, J = 15.6 Hz), 7.44 (1H, d, J = 8.1 Hz), 7.39 (1H, d, J = 2.7 Hz), 7.13 (1H, t, J = 7.9 Hz), 6.81 (1H, d, J = 8.9 Hz), 6.78-6.68 (3H, m), 6.33 (1H, d, J = 15.6 Hz), [symbol] .91 (1H, t, J = 5.5 Hz), 4. [symbol] 3 (2H, q, J = 7.0 Hz), 3.96 (3H, s), 3.75 (3H, s), 3.69-3.61 (2H, m), 2. [symbol] 0 (2H, t, J = 6.6 Hz), 1.45 (3H, t, J = 7.0 Hz). |

[symbol]

indicates data missing or illegible when filed

### PRODUCTION EXAMPLES

### Production Example 1: Synthesis of 3-[5-[2-[[2-(5-fluoropyridin-3-yl)-8,8-dimethyl-7H-purino[8,9-b][1,3]oxazol-4-yl]amino]ethyl]-2-hydroxyphenyl]benzonitrile (Compound A5)

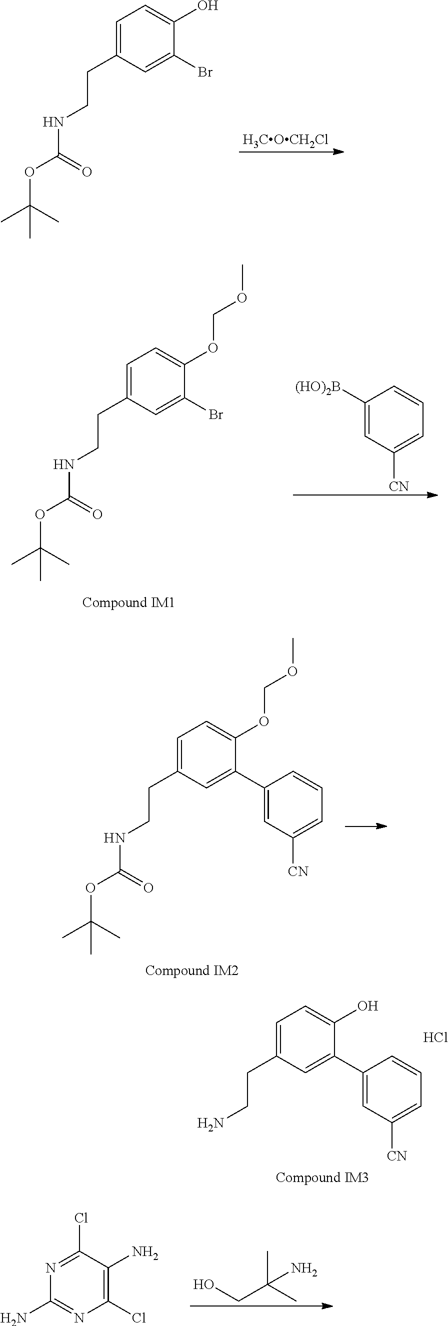

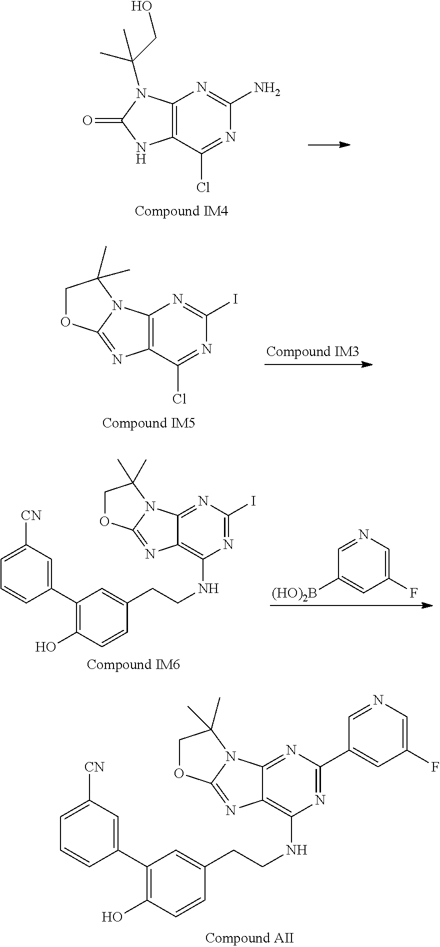 [SMILES: CC(C)(CO)n1c(=O)[nH]c2c(Cl)nc(N)nc21 ; CC1(C)COc2nc3c(Cl)nc(I)nc3n21 ; CC1(C)COc2nc3c(NCCc4ccc(O)c(-c5cccc(C#N)c5)c4)nc(I)nc3n21 ; OB(O)c1cncc(F)c1 ; CC1(C)COc2nc3c(NCCc4ccc(O)c(-c5cccc(C#N)c5)c4)nc(-c4cncc(F)c4)nc3n21]

### (1) Synthesis of tert-butyl N-[2-[3-bromo-4-(methoxymethoxy)phenyl]ethyl]carbamate (Compound IM1)

To a solution of tert-butyl N-[2-(3-bromo-4-hydroxyphenyl)ethyl]carbamate (9.40 g) in DCM (150 ml) were added DIPEA (7.79 ml) and chloromethyl methyl ether (2.94 ml) at 0° C., and the mixture was stirred at room temperature for 3 days. The reaction mixture was concentrated, and the residue was then purified by column chromatography (Hexane/AcOEt) to obtain Compound IM1 (10.9 g).

NMR2(500 MHz); 7.38 (1H, d, J=1.9 Hz), 7.11-7.03 (2H, m), 5.22 (2H, s), 4.53 (1H, s), 3.52 (3H, s), 3.37-3.30 (2H, m), 2.72 (2H, t, J=7.0 Hz), 1.44 (9H, s).

### (2) Synthesis of tert-butyl N-[2-[3-(3-cyanophenyl)-4-(methoxymethoxy)phenyl]ethyl]carbamate (Compound IM2)

A mixture of Compound IM1 (350 mg), 3-cyanophenylboronic acid (186 mg), K₃PO₄ (412 mg), Pd (dppf) Cl₂·DCM (39.7 mg), and 1,4-dioxane/water (4/1) (5 ml) was stirred at 90° C. for 4 hours under nitrogen atmosphere. The reaction mixture was concentrated, and the residue was then purified by column chromatography (Hexane/AcOEt) to obtain Compound IM2 (366 mg).

NMR2 (500 MHz); 7.83 (1H, t, J=1.7 Hz), 7.74 (1H, dt, J=7.9, 1.5 Hz), 7.61 (1H, dt, J=7.7, 1.4 Hz), 7.51 (1H, t, J=7.8 Hz), 7.19-7.15 (2H, m), 7.12 (1H, s), 5.13 (2H, s), 4.57 (1H, s), 3.41-3.34 (5H, m), 2.79 (2H, t, J=7.1 Hz), 1.43 (9H, s)

### (3) Synthesis of 3-[5-(2-aminoethyl)-2-hydroxyphenyl]benzonitrile hydrochloride (Compound IM3)

To a solution of Compound IM2 (364 mg) in EtOH (2 ml) was added 4 N HCl/AcOEt (2 ml), and the mixture was stirred at room temperature for 7 hours. The reaction mixture was concentrated to obtain Compound IM3 (242 mg).

NMR1(500 MHz); 9.81 (1H, s), 7.99 (1H, t, J=1.8 Hz), 7.97-7.88 (4H, m), 7.77 (1H, dt, J=7.7, 1.4 Hz), 7.62 (1H, t, J=7.8 Hz), 7.24 (1H, d, J=2.2 Hz), 7.11 (1H, dd, J=8.3, 2.3 Hz), 6.96 (1H, d, J=8.3 Hz), 3.09-2.98 (2H, m), 2.86-2.79 (2H, m).

### (4) Synthesis of 2-amino-6-chloro-9-(1-hydroxy-2-methylpropan-2-yl)-7H-purin-8-one (Compound IM4)

A solution of 2,5-diamino-4,6-dichloropyrimidine (10.0 g) and 2-amino-2-methyl-1-propanol (11.7 ml) in NMP (10 ml) was stirred overnight at 140° C. The reaction mixture was purified by column chromatography (Hexane/AcOEt/MeOH). To a solution of the product in THF (150 ml) was added CDI (19.9 g) at 0° C., and the mixture was stirred for 1 hour. To the mixture were added 50% MeOH aqueous solution (300 ml) and 5 N NaOH aqueous solution (44.7 ml), and the mixture was stirred for 1 hour. The reaction mixture was concentrated, 5 N HCl aqueous solution was added to the residue, and the solid precipitate was collected by filtration to obtain Compound IM4 (10.9 g).

NMR1(500 MHz); 11.16 (1H, s), 6.48 (2H, s), 4.87 (1H, t, J=6.6 Hz), 3.79 (2H, d, J=6.6 Hz), 1.60 (6H, s).

### (5) Synthesis of 4-chloro-2-iodo-8,8-dimethyl-7H-purino[8,9-b][1,3]oxazole (Compound IM5)

To a suspension solution of Compound IM4 (10.90 g) and triphenylphosphine (13.31 g) in THF (200 ml) was added dropwise diisopropyl azodicarboxylate (40% toluene solution) (26.7 ml) at 0° C. under nitrogen atmosphere, and the mixture was stirred for 2 hours. The reaction mixture was concentrated, and the residue was purified by column chromatography (Hexane/AcOEt). To a solution of the product in THF (200 ml) were added copper(I) iodide (8.06 g), diiodomethane (10.24 ml), and tert-butyl nitrite (7.55 ml), and the mixture was stirred at 60° C. for 5 hours. The reaction mixture was filtered through Celite, and the filtrate was concentrated. The residue was purified by column chromatography (Hexane/AcOEt) to obtain Compound IM5 (9.29 g).

NMR1 (500 MHz); 5.02 (2H, s), 1.68 (6H, s).

### (6) Synthesis of 3-[2-hydroxy-5-[2-[(2-iodo-8,8-dimethyl-7H-purino[8,9-b][1,3]oxazol-4-yl)amino]ethyl]phenyl]benzonitrile (Compound IM6)

A suspension of Compound IM5 (150 mg), 3-[5-(2-aminoethyl)-2-hydroxyphenyl]benzonitrile hydrochloride (153 mg), and DIPEA (0.22 ml) in IPA (2 ml) was stirred overnight at 80° C. Water was added to the mixture, and the solid precipitate was collected by filtration to obtain Compound IM6 (211 mg).

NMR1 (500 MHz); 9.62 (1H, s), 7.95 (1H, s), 7.88 (1H, d, J=7.9 Hz), 7.75 (1H, d, J=7.7 Hz), 7.67 (1H, s), 7.60 (1H, t, J=7.8 Hz), 7.22 (1H, s), 7.08 (1H, dd, J=8.3, 2.2 Hz), 6.88 (1H, d, J=8.2 Hz), 4.85 (2H, s), 3.92-3.51 (2H, m), 2.80 (2H, t, J=7.3 Hz), 1.60 (6H, s).

### (7) Synthesis of Compound A5

A mixture of Compound IM6 (244 mg), 5-fluoropyridine-3-boronic acid (93 mg), Pd(dppf)Cl₂·DCM (18.0 mg), K₃PO₄ (188 mg), and 1,4-dioxane/water (4/1) (1 ml) was stirred at 90° C. for 3 hours under nitrogen atmosphere. The reaction mixture was purified by column chromatography (Hexane/AcOEt). The product was washed with Hexane/AcOEt to obtain Compound A5 (197 mg).

NMR1 (500 MHz); 9.58 (1H, s), 9.34 (1H, s), 8.61 (1H, d, J=2.9 Hz), 8.37-8.30 (1H, m), 7.90 (1H, s), 7.84 (1H, d, J=7.9 Hz), 7.73 (1H, dt, J=7.8, 1.4 Hz), 7.59-7.52 (2H, m), 7.24 (1H, s), 7.12 (1H, dd, J=8.2, 2.2 Hz), 6.87 (1H, d, J=8.2 Hz), 4.91 (2H, s), 3.79 (2H, s), 2.90 (2H, t, J=7.2 Hz), 1.71 (6H, s).

### Production Example 2: Synthesis of 2-(2-Fluorophenyl)-4-[2-[[2-(5-fluoropyridin-3-yl)-8,8-dimethyl-7H-purino[8,9-b][1,3]oxazol-4-yl]amino]ethyl]phenol (Compound A6)

The object compound was synthesized in the substantially same method as the compound A5 except that 3-cyanophenylboronic acid was changed to 2-fluorophenylboronic acid in the method for synthesizing the compound IM2.

NMR1(500 MHz); 9.37-9.31 (2H, m), 8.62 (1H, d, J=2.8 Hz), 8.38-8.31 (1H, m), 7.53 (1H, s), 7.39-7.31 (1H, m), 7.31-7.25 (1H, m), 7.21-7.10 (3H, m), 7.10-7.03 (1H, m), 6.84 (1H, d, J=8.2 Hz), 4.91 (2H, s), 3.77 (2H, s), 2.88 (2H, t, J=7.4 Hz), 1.71 (6H, s).

### Production Example 3: Synthesis of 2-(5-Fluoropyridin-3-yl)-4-[2-[[2-(5-fluoropyridin-3-yl)-8,8-dimethyl-7H-purino[8,9-b][1,3]oxazol-4-yl]amino]ethyl]phenol (Compound A7)

The object compound was synthesized in the substantially same method as the compound A5 except that 3-cyanophenylboronic acid was changed to 5-fluoropyridine-3-boronic acid in the method for synthesizing compound IM2.

NMR1 (500 MHz); 9.69 (1H, s), 9.32 (1H, s), 8.61 (1H, d, J=2.9 Hz), 8.56 (1H, s), 8.47 (1H, d, J=2.8 Hz), 8.36-8.30 (1H, m), 7.80-7.74 (1H, m), 7.54 (1H, s), 7.27 (1H, s), 7.15 (1H, dd, J=8.3, 2.2 Hz), 6.88 (1H, d, J=8.2 Hz), 4.90 (2H, s), 3.80 (2H, s), 2.91 (2H, t, J=7.2 Hz), 1.71 (6H, s).

### Production Example 4: Synthesis of 2-(2-Fluorophenyl)-4-[2-[[2-(5-fluoropyridin-3-yl)-8,8-dimethyl-7H-purino[8,9-b][1,3]thiazol-4-yl]amino]ethyl]phenol (Compound A8)

### (1) Synthesis of 4-chloro-2-iodo-8,8-dimethyl-7H-purino[8,9-b][1,3]thiazole (Compound IM5′)

A solution of 2,5-diamino-4,6-dichloropyrimidine (10.0 g) and 2-amino-2-methyl-1-propanol (12.8 ml) was stirred at 140° C. for 4 hours. Water was added to the solution at room temperature, and the solid precipitated was collected by filtration. TCDI (20.5 g) was gradually added to the solution of the solid collected by filtration in THF (100 ml) at 0° C., and the mixture was stirred at room temperature for 1 hour. After concentrating the reaction solution, water was added at 0° C., and the solid precipitated was collected by filtration. A suspension of the solid collected by filtration, copper (I) iodide (4.19 g), diiodomethane (7.09 ml), tert-butyl nitrite (3.93 ml) in THF (80 ml) was stirred overnight at 60° C. The reaction mixture was filtered through Celite, and the filtrate was concentrated. The residue was purified by column chromatography (Hexane/AcOEt), and then washed with IPA to obtain the object compound (3.96 g).

NMR1 (500 MHz); 3.95 (2H, s), 1.72 (6H, s).

### (2) Synthesis of Compound A8

The object compound was synthesized in the substantially same manner as the compound A5 except that 3-cyanophenylboronic acid was changed to 2-fluorophenylboronic acid in the method for synthesizing the compound IM2, and the compound IM5 was changed to the compound IM5′.

NMR1 (500 MHz); 9.35 (1H, s), 9.33 (1H, s), 8.63 (1H, d, J=2.9 Hz), 8.38-8.32 (1H, m), 7.81 (1H, s), 7.38-7.30 (1H, m), 7.27-7.23 (1H, m), 7.21-7.10 (3H, m), 7.07 (1H, s), 6.84 (1H, d, J=8.2 Hz), 3.90 (2H, s), 3.78 (2H, s), 2.89 (2H, t, J=7.4 Hz), 1.78 (6H, s).

### TEST EXAMPLE

### Test Example 1 (Platelet Production: Shake Culturing)

The immortalized megakaryocyte cell line obtained according to the method described in WO 2016/204256 was washed twice with D-PBS(−) and then cultured in medium not containing doxycycline to terminate forced expression (cultured under conditions where gene expression is OFF). Shake culturing at 100 rpm was performed in the following medium after the cells were seeded in a 125-mL polycarbonate Erlenmeyer flask (Corning #431143) at 25 mL/flask and a seeding density of 1×10⁵ cells/mL. Culturing conditions were 37° C. and 5% CO₂.

The medium was obtained by adding the following components to IMDM serving as the basal medium (concentrations indicate final concentrations).
FBS 15%
L-Glutamine 2 mM
ITS 100-fold dilution
MTG 450 μM
Ascorbic acid 50 μg/mL
SCF 50 ng/mL
TA-316 0.1 μg/mL
ADAM inhibitor 15 μM
ROCK inhibitor 0.5 μM

Culturing was initiated by adding an aryl hydrocarbon receptor antagonist (Compound A5, final concentration: 0.1 μM) or DMSO (Control) to the medium at the same time as seeding the cells. The compound of the present invention (Examples 1 to 61, final concentration: 10 μM) was added to the medium at Day 3 after initiation of the culturing. After culturing for 6 days in total, the number of platelets was measured. The measurement method was as follows. The same operation was performed for control.

At 6 days after initiation of the culturing under conditions where gene expression was OFF, a part of the culture supernatant was collected, and suspended with the following antibody and Flow-Count Fluorospheres (Beckman Coulter #7547053) to perform staining.
APC-labeled anti-CD41 antibody (BioLegend #303710)
eFluor 450-labeled anti-CD42a antibody (eBioscience #48-0428-42)
PE-labeled anti-CD42b antibody (BioLegend #303906)

At 30 minutes after staining, number of platelets (CD41, CD42a and CD42b-positive cells) was counted by using FACSVerse (manufactured by BD Japan) with Flow-Count Fluorospheres. The number of platelets was given as a percentage of the control.

Table 3 shows the results of culturing with DMSO added at the same time as cell seeding, and Table 4 shows the results of culturing with an aryl hydrocarbon receptor antagonist added at the same time as cell seeding.

In the Tables, + and ++ indicate the amount of platelets production increased by not less than 1.5 times and less than 6.5 times, and not less than 6.5 times, respectively, as compared to the control.

The compounds of Examples 60 and 61 are known compounds, and were manufactured by a method described in WO 2019/167973.

| | |
|---|---|
| Example 60 | 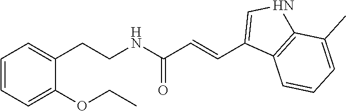 [SMILES: CCOc1ccccc1CCNC(=O)/C=C/c1c[nH]c2c(C)cccc12] |
| Example 61 | 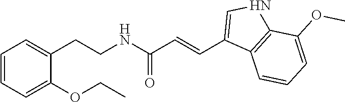 [SMILES: CCOc1ccccc1CCNC(=O)/C=C/c1c[nH]c2c(OC)cccc12] |

TABLE 3

| | Amount of platelet |
|---|---|
| Example | production (vs. Control) |
| 1 | + |
| 2 | + |
| 3 | + |
| 4 | + |
| 5 | + |
| 6 | + |
| 7 | + |
| 8 | + |
| 9 | + |
| 10 | + |
| 11 | + |
| 12 | + |
| 13 | + |
| 14 | + |
| 15 | ++ |
| 16 | + |
| 17 | + |
| 18 | + |
| 19 | + |
| 20 | + |
| 21 | + |
| 22 | + |
| 23 | + |
| 24 | + |
| 25 | + |
| 26 | + |
| 27 | + |
| 28 | + |
| 29 | + |
| 30 | + |
| 31 | + |
| 32 | + |
| 33 | + |
| 34 | + |
| 35 | + |
| 36 | + |
| 37 | + |
| 38 | + |
| 39 | + |
| 40 | + |
| 41 | + |
| 42 | + |
| 43 | + |
| 44 | + |
| 45 | + |
| 46 | + |
| 47 | + |
| 48 | + |
| 49 | + |
| 50 | + |
| 51 | + |
| 52 | + |
| 53 | + |
| 54 | + |
| 55 | + |
| 56 | + |
| 57 | + |
| 58 | + |
| 59 | + |
| 60 | + |
| 61 | + |

TABLE 4

| | Amount of platelet |
|---|---|
| Example | production (vs. Control) |
| 1 | ++ |
| 2 | ++ |
| 3 | ++ |
| 4 | ++ |
| 5 | ++ |
| 6 | ++ |
| 7 | ++ |
| 8 | ++ |
| 9 | ++ |
| 10 | ++ |
| 11 | ++ |
| 12 | ++ |
| 13 | ++ |
| 14 | ++ |
| 15 | ++ |
| 16 | ++ |
| 17 | ++ |
| 18 | ++ |
| 19 | ++ |
| 20 | ++ |
| 21 | ++ |
| 22 | ++ |
| 23 | ++ |
| 24 | ++ |
| 25 | ++ |
| 26 | ++ |
| 27 | ++ |
| 28 | ++ |
| 29 | ++ |
| 30 | ++ |
| 31 | ++ |
| 32 | ++ |
| 33 | ++ |
| 34 | ++ |
| 35 | ++ |
| 36 | ++ |
| 37 | ++ |
| 38 | + |
| 39 | ++ |
| 40 | ++ |
| 41 | ++ |
| 42 | ++ |
| 43 | ++ |
| 44 | ++ |
| 45 | ++ |
| 46 | ++ |
| 47 | ++ |
| 48 | ++ |
| 49 | ++ |
| 50 | ++ |
| 51 | ++ |
| 52 | ++ |
| 53 | ++ |
| 54 | ++ |
| 55 | ++ |
| 56 | ++ |
| 57 | ++ |
| 58 | ++ |
| 59 | ++ |
| 60 | ++ |
| 61 | ++ |

### Test Example 2 (Platelet Production: Shake Culturing)

Culturings were performed in the same manner as in Test Example 1 by using the compounds of Examples 57 to 61 and adding Compound A1 (final concentration: 0.75 μM), Compound A2 (final concentration: 0.1 μM), Compound A3 (final concentration: 10 μM), Compound A4 (final concentration: 1 μM) and Compounds A6 to A8 (final concentration: 0.1 μM) as aryl hydrocarbon receptor antagonist. The results are shown in the following Table 5, together with the results of comparative examples wherein culturings were performed by using only an aryl hydrocarbon receptor antagonist.

TABLE 5

| | | Aryl hydrocarbon | Amount of platelet |
|---|---|---|---|
| | | receptor | production |
| Example | | antagonist | (vs. Control) |
| 62 | Compound of | Compound A2 | ++ |
| | Example 60 | | |
| 63 | Compound of | Compound A1 | ++ |
| | Example 61 | | |
| 64 | Compound of | Compound A2 | ++ |
| | Example 61 | | |
| 65 | Compound of | Compound A3 | ++ |
| | Example 61 | | |
| 66 | Compound of | Compound A4 | ++ |
| | Example 61 | | |
| 67 | Compound of | Compound A [symbol] | ++ |
| | Example 61 | | |
| 68 | Compound of | Compound A7 | ++ |
| | Example 61 | | |
| 69 | Compound of | Compound A8 | ++ |
| | Example 61 | | |
| 70 | Compound of | Compound A2 | ++ |
| | Example [symbol] 7 | | |
| 71 | Compound of | Compound A2 | ++ |
| | Example [symbol] 8 | | |
| 72 | Compound of | Compound A2 | ++ |
| | Example 59 | | |
| Comparative | None | Compound A1 | + |
| example 1 | | | |
| Comparative | None | Compound A2 | + |
| example 2 | | | |
| Comparative | None | Compound A3 | + |
| example 3 | | | |
| Comparative | None | Compound A4 | + |
| example 4 | | | |
| Comparative | None | Compound A5 | + |
| example 5 | | | |
| Comparative | None | Compound A6 | + |
| example 6 | | | |
| Comparative | None | Compound A7 | + |
| example 7 | | | |
| Comparative | None | Compound A8 | + |
| example 8 | | | |

[symbol]

indicates data missing or illegible when filed

1. A compound represented by general formula [I]: 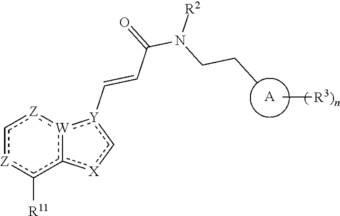
wherein
R¹¹ is hydrogen, halogen, —C₁₋₆ alkyl or —O—C₁₋₆ alkyl;
R² is hydrogen or —C₁₋₆ alkyl,
R³ is halogen, -Qₖ-(C₁₋₆ alkyl)ₘ-Qₚ-R³¹, optionally-substituted phenyl or optionally-substituted heteroaryl which is selected from the groupe consisting of furyl, thienyl, oxazolyl, thiazolyl, pyrazolyl, pyridyl, pyrazyl, pyridazinyl and pyrimidyl,
R³¹ is —C₁₋₆ alkyl or —C₃₋₈ cycloalkyl,
Qs are the same or different and each independently represent oxygen, sulfur, —C(═O)—O— or —NH—,
k, m and p are 0 or 1,
n is 0, 1 or 2, wherein when n is 2, R³s each independently represent the same or different substituent,
W is carbon or nitrogen,
X is carbon, nitrogen or N—R¹²,
Y is carbon or nitrogen,
Zs are the same or different and each independently represent nitrogen or C—H,
provided that X and Y are not carbon at the same time,
R¹² is hydrogen, —C₁₋₆ alkyl, —C₁₋₆ alkyl-O—C₁₋₆ alkyl, —C(═O)—C₁₋₆ alkyl, —C(═O)-aryl or —C(═O)—O—C₁₋₆ alkyl,
Ring A is aryl or heteroaryl, [symbol]
is single bond or double bond;
provided that when X is N—H, W and Y are carbon and all Z are C—H, Ring A is neither 2-(-O—C₁₋₆ alkyl)phenyl nor 2,5-di(—O—C₁₋₆ alkyl)phenyl,
or a salt thereof.

2. The compound according to claim 1, wherein in the general formula [I], 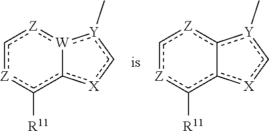
wherein R¹¹, W, X, Y, Zs and [symbol] are as defined above,
or a salt thereof.

3. The compound according to claim 1, wherein in the general formula [I], 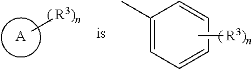
wherein R³ and n are as defined above,
or a salt thereof.

4. The compound according to claim 1, wherein in the general formula [I], the heteroaryl in Ring A is selected from the group consisting of furan, thiophene, pyridine and quinoline, or a salt thereof.

5. The compound according to claim 1, wherein in the general formula [I], 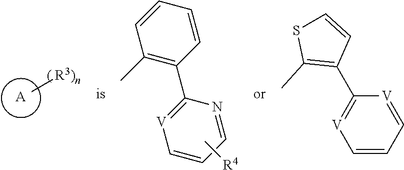
wherein Vs are the same or different and each independently represent nitrogen or C—H, R⁴ is hydrogen, halogen, —C₁₋₆ alkyl or —O—C₁₋₆ alkyl,
or a salt thereof.

6. The compound according to claim 1, which is represented by general formula [Ia]: 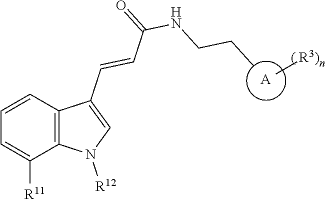
wherein R¹¹ is hydrogen, halogen, —C₁₋₆ alkyl or —O—C₁₋₆ alkyl,
R¹² is hydrogen or —C(═O)—O—C₁₋₆ alkyl, 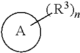
is pyridylbenzene, pyrimidylbenzene (wherein the pyrimidyl is optionally substituted by halogen, —C₁₋₆ alkyl or —O—C₁₋₆ alkyl), phenylthiophene, pyridylthiophene or pyrimidylthiophene, or a salt thereof.

7. The compound according to claim 1, which is selected from the group consisting of the following compounds: 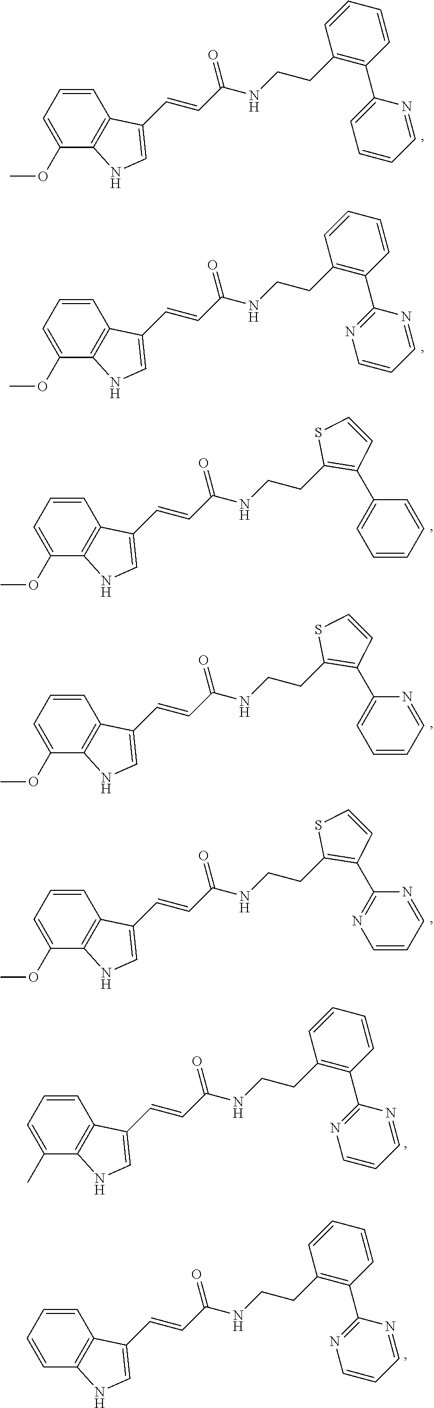 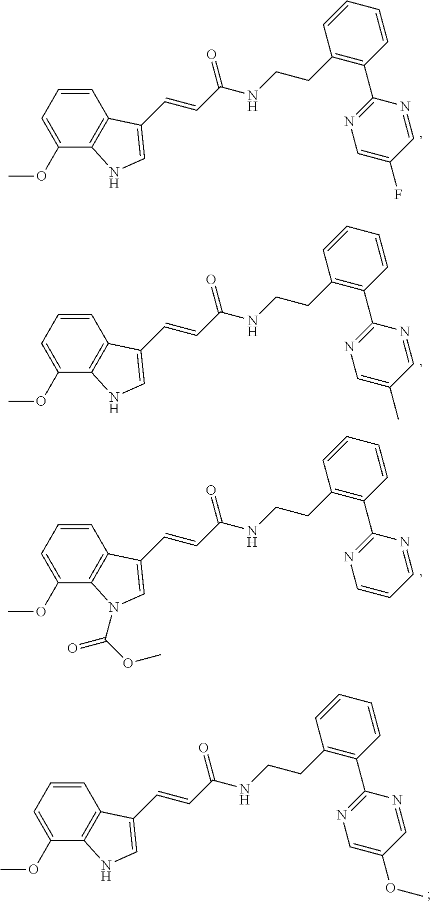
or a salt thereof.

8. A platelet production promoting agent comprising a compound represented by general formula [I′]: 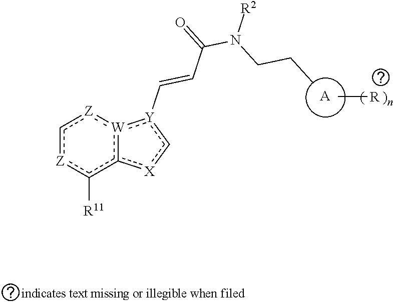
wherein
R¹¹ is hydrogen, halogen, —C₁₋₆ alkyl or —O—C₁₋₆ alkyl;
R² is hydrogen or —C₁₋₆ alkyl,
R³ is halogen, -Qₖ-(C₁₋₆ alkyl)ₘ-Qₚ-R³¹, optionally-substituted phenyl or optionally-substituted heteroaryl which is selected form the group consisting of furyl, thienyl, oxazolyl, thiazolyl, pyrazolyl, pyridyl, pyrazyl, pyridazinyl and pyrimidyl,
R³¹ is —C₁₋₆ alkyl or —C₃₋₈ cycloalkyl,
Qs are the same or different and each independently represent oxygen, sulfur, —C(═O)—O— or —NH—,
k, m and p are 0 or 1,
n is 0, 1 or 2, wherein when n is 2, R³s each independently represent the same or different substituent
W is carbon or nitrogen,
X is carbon, nitrogen or N—R¹²,
Y is carbon or nitrogen,
Zs are the same or different and each independently represent nitrogen or C—H,
provided that X and Y are not carbon at the same time
R¹² is hydrogen, —C₁₋₆ alkyl, —C₁₋₆ alkyl-O—C₁₋₆ alkyl, —C(═O)—C₁₋₆ alkyl, —C(═O)-aryl or —C(═O)—O—C₁₋₆ alkyl,
Ring A is aryl or heteroaryl, [symbol]
is single bond or double bond,
or a salt thereof.

9. The promoting agent according to claim 8, which is for use in combination with an aryl hydrocarbon receptor antagonist.

10. The promoting agent according to claim 8, wherein the aryl hydrocarbon receptor antagonist is selected from the group consisting of the following compounds:

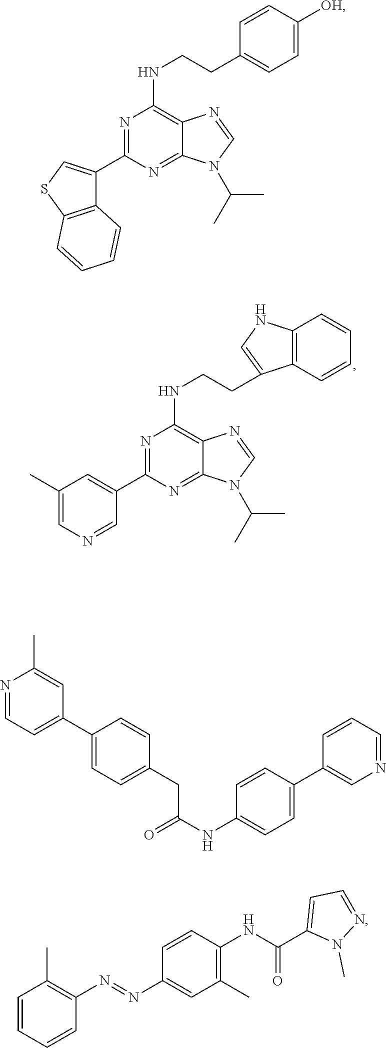 [SMILES: CC(C)n1cnc2c(NCCc3ccc(O)cc3)nc(-c3csc4ccccc34)nc21 ; Cc1cncc(-c2nc(NCCc3c[nH]c4ccccc34)c3ncn(C(C)C)c3n2)c1 ; Cc1cc(-c2ccc(CC(=O)Nc3ccc(-c4cccnc4)cc3)cc2)ccn1 ; Cc1ccccc1/N=N/c1ccc(NC(=O)c2ccnn2C)c(C)c1]

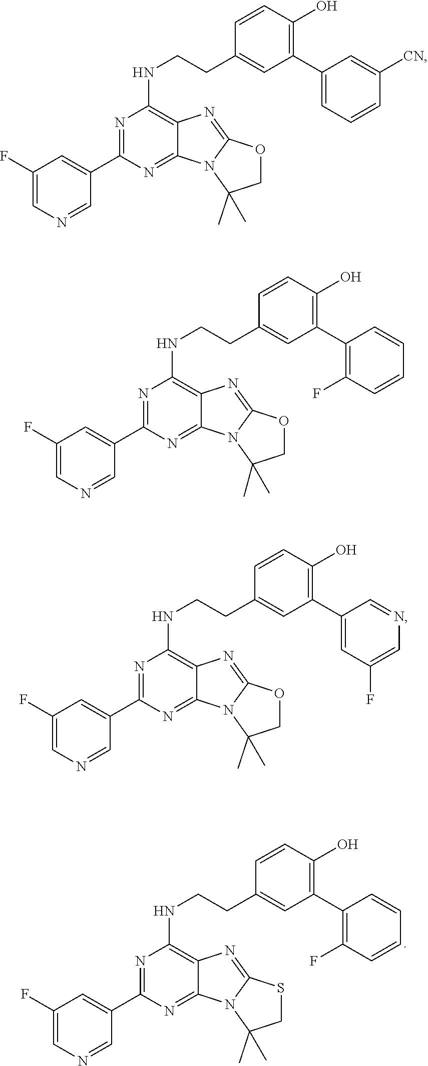 [SMILES: CC1(C)COc2nc3c(NCCc4ccc(O)c(-c5cccc(C#N)c5)c4)nc(-c4cncc(F)c4)nc3n21 ; CC1(C)COc2nc3c(NCCc4ccc(O)c(-c5ccccc5F)c4)nc(-c4cncc(F)c4)nc3n21 ; CC1(C)COc2nc3c(NCCc4ccc(O)c(-c5cncc(F)c5)c4)nc(-c4cncc(F)c4)nc3n21 ; CC1(C)CSc2nc3c(NCCc4ccc(O)c(-c5ccccc5F)c4)nc(-c4cncc(F)c4)nc3n21]

11. Use of the compound according to claim 8 or a salt thereof for promoting platelet production.

12. The compound according to claim 8 or a salt thereof for use in promoting platelet production.

13. A method for promoting platelet production, which comprises culturing platelet progenitor cells in the presence of the compound according to claim 8 or a salt thereof.

14. A method for producing platelets, which comprises culturing platelet progenitor cells in the presence of the compound according to claim 8 or a salt thereof.

15. A method for culturing platelet progenitor cells to promote platelet production, which comprises culturing platelet progenitor cells in the presence of the compound according to claim 8 or a salt thereof.
